(19) United States
(12) Patent Application Publication
(10) Pub. No.: US 2011/0201496 A1
(43) Pub. Date: Aug. 18, 2011

(54) Substituted 5-Hetaryl-4-Aminopyrimidines

(76) Inventors: Joachim Rheinheimer, Ludwigshafen (DE); Thomas Grote, Wachenheim (DE); Bernd Müller, Frankenthal (DE); Wassilios Grammenos, Ludwigshafen (DE); Sarah Ulmschneider, Bad Durkheim (DE); Jens Renner, Bad Durkheim (DE); Reinhard Stierl, Kaohsiung County (TW)

(21) Appl. No.: 12/293,432

(22) PCT Filed: Mar. 26, 2007
(86) PCT No.: PCT/EP2007/052888

§ 371 Date: Sep. 18, 2008

(30) Foreign Application Priority Data
Mar. 27, 2006 (EP) 06006255.1

(51) Int. Cl. A01N 43/54; C07D 401/04; A01C 1/06; A61K 31/506; A01P 3/00; A61P 35/00

(52) U.S. Cl. 504/100; 544/328; 514/256

(57) ABSTRACT

The present invention relates to the use of 5-hetaryl-4-aminopyrimidines of the formula I and their salts for controlling plant-damaging fungi. The invention also relates to novel 5-hetaryl-4-aminopyrimidines and to crop protection compositions comprising at least one such compound as active component.

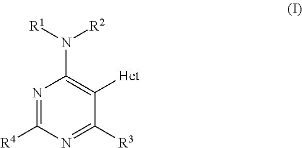

Het is an optionally substituted 5- or 6-membered aromatic heterocycle which has 1, 2, 3 or 4 heteroatoms selected from the group consisting of nitrogen, oxygen and sulfur as ring members, where the 5- or 6-membered heteroaromatic radical may have 1, 2, 3 or 4 identical or different substituents L,
R¹, R² are inter alia hydrogen, C₁-C₈-alkyl, C₃-C₈-cycloalkyl, C₅-C₁₀-bicycloalkyl, C₂-C₈-alkenyl, C₄-C₁₀-alkadienyl, C₃-C₆-cycloalkenyl, C₂-C₈-alkynyl, phenyl, naphthyl or a five- or six-membered saturated, partially unsaturated or aromatic heterocycle which has one, two, three or four heteroatoms from the group consisting of O, N or S as ring members; or together form a ring;
R³ is inter alia hydrogen, OH, halogen, cyano, NR³¹R³², C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, and
R⁴ is halogen, cyano, hydroxyl, mercapto, N₃, C₁-C₆-alkyl, C₂-C₈-alkenyl, C₂-C₈-alkynyl, C₁-C₆-haloalkyl, C₁-C₆-alkoxy, C₃-C₈-alkenyloxy, C₃-C₈-alkynyloxy, C₁-C₆-haloalkoxy, C₁-C₆-alkylthio, C₃-C₈-alkenylthio, C₃-C₈-alkinylthio, C₁-C₆-haloalkylthio, or is a radical of the formula C(═Z)OR⁴¹, C(═Z)NR⁴²R⁴³, C(═Z)NR⁴⁴—NR⁴²R⁴³, C(═Z)R⁴⁵, CR⁴⁶R⁴⁷—OR⁴⁸, CR⁴⁶R⁴⁷—NR⁴²R⁴³, ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR^{52a}(N═CR⁴⁹R⁵⁰), NR⁵²NR⁴²R⁴³, NR⁵²OR⁴¹ or C(═N—X—R⁴⁵)SR⁴¹.

The present invention relates to the use of 5-hetaryl-4-aminopyrimidines for controlling plant-damaging fungi, to novel 5-hetaryl-4-aminopyrimidines and to crop protection compositions comprising at least one such compound as active component.

5-Phenyl-4-aminopyrimidines and their use for controlling plant-damaging fungi (phytopathogenic fungi) are known from WO 01/96314, WO 02/074753, WO 03/070721, WO 03/043993, WO 2004/103978 and WO 2005/019187. Some of the 5-phenyl-4-aminopyrimidines known from the prior art are, with respect to their fungicidal action, unsatisfactory, or they have unwanted properties, such as poor compatibility with crop plants.

WO 2006/029867 describes 5-heterocyclyl-4-aminopyrimidines having a heterocyclic radical in the 2-position of the pyrimidine ring. The fungicidal activity of the compounds described in this publication is unsatisfactory.

Accordingly, it is the object of the present invention to provide compounds having improved fungicidal activity and/or better crop plant compatibility.

Surprisingly, this object is achieved by 5-hetaryl-4-aminopyrimidines of the formula I defined below, and by the agriculturally acceptable salts of the compounds I.

Accordingly, the present invention relates to the use of 5-hetaryl-4-aminopyrimidine compounds of the formula I

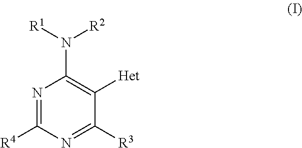

in which
Het is a 5- or 6-membered heteroaromatic radical which has 1, 2, 3 or 4 heteroatoms selected from the group consisting of nitrogen, oxygen and sulfur as ring members, where the 5- or 6-membered heteroaromatic radical may have 1, 2, 3 or 4 identical or different substituents L, where
   L is selected from the group consisting of halogen, cyano, hydroxyl, cyanato (OCN), nitro, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₈-cycloalkenyl, C₃-C₆-cycloalkoxy, C₁-C₈-alkoximinoalkyl, C₂-C₁₀-alkenyloximinoalkyl, C₂-C₁₀-alkynyloximinoalkyl, C₂-C₁₀-alkynylcarbonyl, C₃-C₆-cycloalkylcarbonyl, NR⁵R⁶, NR⁵—C(═O)—R⁶, NR⁵—C(═S)—R⁶, S(═O)ₙA¹, C(═O)A², C(═S)A², a group —C(═N—OR⁷)A³, a group —C(═N—NR⁸R⁹)A⁴, phenyl and a five-, six-, seven-, eight-, nine- or ten-membered saturated, partially unsaturated or aromatic heterocycle which has one, two, three or four heteroatoms from the group consisting of O, N and S as ring members and in which phenyl and the heterocycle are unsubstituted or may have 1, 2, 3 or 4 substituents selected from the group consisting of halogen, nitro, cyano, OH, C₁-C₂-alkyl, C₁-C₂-haloalkyl, C₁-C₂-alkoxy, C₁-C₂-haloalkoxy, C₁-C₄-alkoxycarbonyl, C₁-C₄-alkylcarbonyl, amino, C₁-C₄-alkylamino and di-C₁-C₄-alkylamino,
      in which
      R⁵, R⁶ independently of one another are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, where the 5 last-mentioned radicals may be partially or fully halogenated and/or may carry one, two, three or four radicals selected from the group consisting of cyano, C₁-C₄-alkoximino, C₂-C₄-alkenyloximino, C₂-C₄-alkynyloximino or C₁-C₄-alkoxy;
      A¹ is hydrogen, hydroxyl, C₁-C₈-alkyl, amino, C₁-C₈-alkylamino or di-(C₁-C₈-alkyl)amino;
      n is 0, 1 or 2;
      A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹;
      A³ and A⁴ independently of one another are C₁-C₈-alkyl, C₂-C₈-alkenyl, C₁-C₈-haloalkyl, C₂-C₈-haloalkenyl, C₃-C₆-cycloalkyl, C₁-C₈-alkoxy, C₁-C₆-haloalkoxy, C₂-C₁₀-alkenyloxy, C₂-C₁₀-alkynyloxy or a group NR¹⁰R¹¹;
      R⁷, R⁸, R⁹, R¹⁰ and R¹¹ independently of one another are selected from the group consisting of hydrogen, C₁-C₆-alkyl, C₃-C₆-cycloalkyl, C₂-C₆-alkenyl and C₂-C₆-alkynyl, where the four last-mentioned radicals may have one, two, three, four, five or six radicals R^{a}; or
         R⁸ and R⁹ and/or R¹⁰ and R¹¹ together with the nitrogen atom to which they are attached form a four-, five- or six-membered saturated or partially unsaturated ring which may carry one, two or three or four substituents independently of one another selected from R^{a};
         R^{a} is halogen, OH, C₁-C₈-alkyl or C₁-C₈-alkoxy;
R¹ is hydrogen, C₁-C₈-alkyl, C₃-C₈-cycloalkyl, C₅-C₁₀-bicycloalkyl, C₂-C₈-alkenyl, C₄-C₁₀-alkadienyl, C₃-C₆-cycloalkenyl, C₂-C₈-alkynyl, phenyl, naphthyl or a five- or six-membered saturated, partially unsaturated or aromatic heterocycle which is attached via carbon and which has one, two, three or four heteroatoms from the group consisting of O, N and S as ring members;
R² has one of the meanings given for R¹ and may also be one of the following radicals: NH₂, C₁-C₈-alkoxy, C₃-C₈-cycloalkoxy, C₂-C₈-alkenyloxy, C₂-C₈-alkynyloxy, C₁-C₈-alkylamino and also di-C₁-C₈-alkylamino;
   where the radicals R¹ and R² different from hydrogen may also be partially or fully halogenated and/or may carry one, two, three or four identical or different groups R²¹:
   R²¹ is cyano, nitro, hydroxyl, carboxyl, C₁-C₆-alkylcarbonyl, C₃-C₆-cycloalkyl, C₁-C₆-alkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₃-C₈-cycloalkenyl, C₂-C₆-alkenyloxy, C₂-C₆-alkynyl, C₃-C₆-alkynyloxy, C₃-C₆-cycloalkoxy, C₃-C₆-cycloalkenyloxy, oxy-C₁-C₃-alkylenoxy, phenyl, naphthyl, a five-, six-, seven-, eight-, nine- or ten-membered saturated, partially unsaturated or aromatic heterocycle which has one, two, three or four heteroatoms from the group consisting of O, N and S as ring members,
      where the aliphatic, alicyclic, heterocyclic and aromatic groups in R²¹ for their part may be partially or fully halogenated or may carry one, two or three groups R²²:
      R²² is cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
         cycloalkyl, bicycloalkyl, cycloalkoxy, heterocyclyl, heterocyclyloxy, where the cyclic systems contain 3 to 10 ring members, aryl, aryloxy, arylthio, aryl-C₁-C₆-alkoxy, aryl-C₁-C₆-alkyl, hetaryl, hetaryloxy, hetarylthio, where the aryl radicals preferably contain 6, 7, 8, 9 or 10 ring members and the hetaryl radicals 5 or 6 ring members, where the cyclic systems may be partially or fully halogenated or substituted by alkyl or haloalkyl groups;
   R¹ and R² together with the nitrogen atom to which they are attached may also form a five- or six-membered saturated, partially unsaturated or aromatic heterocycle which is attached via N and which may have one, two or three further heteroatoms from the group consisting of O, N and S as ring members and/or may carry one or more substituents from the group consisting of halogen, oxo, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy and/or in which two substituents attached to adjacent ring atoms may be C₁-C₆-alkylene, oxy-C₂-C₄-alkylene or oxy-C₁-C₃-alkylenoxy;
R³ is hydrogen, OH, halogen, cyano, NR³¹R³², C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, where the 7 last-mentioned radicals may be partially or fully halogenated and/or may carry one, two or three substituents selected from the group consisting of nitro, cyano, OH, C₁-C₂-alkoxy, C₁-C₄-alkoxycarbonyl, amino, C₁-C₄-alkylamino and di-C₁-C₄-alkylamino,
   where R³¹ has one of the meanings given for R⁵ and R³² has one of the meanings given for R⁶;
R⁴ is halogen, cyano, hydroxyl, mercapto, N₃, C₁-C₆-alkyl, C₂-C₈-alkenyl, C₂-C₈-alkynyl, C₁-C₆-haloalkyl, C₁-C₆-alkoxy, C₃-C₈-alkenyloxy, C₃-C₈-alkynyloxy, C₁-C₆-haloalkoxy, C₁-C₆-alkylthio, C₃-C₈-alkenylthio, C₃-C₈-alkynylthio, C₁-C₆-haloalkylthio, or is a radical of the formula C(═Z)OR⁴¹, C(═Z)NR⁴²R⁴³, C(═Z)NR⁴⁴—NR⁴²R⁴³, C(═Z)R⁴⁵, CR⁴⁶R⁴⁷—OR⁴⁸, CR⁴⁶R⁴⁷—NR⁴²R⁴³, ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)−NR⁴²R⁴³), NR^{52a}(N═CR⁴⁹R⁵⁰), NR⁵²NR⁴²R⁴³, NR⁵²OR⁴¹ or C(═N—X—R⁴⁵)SR⁴¹ where
   Z is O, S, NR⁵³, NOR⁵⁴ or N—NR⁵⁵R⁵⁶;
   X is a chemical bond, oxygen, a carbonyl group, a group NR⁵² or one of the following groups: —(C═O)—NH— or —(C═O)—O—, where the carbonyl group is attached to the nitrogen atom;
   R⁴¹, R⁴², R⁴³, R⁴⁴, R⁴⁵, R⁴⁶, R⁴⁷, R⁴⁸, R⁴⁹, R⁵⁰, R⁵¹, R⁵², R^{52a}, R⁵³, R⁵⁴, R⁵⁵ and R⁵⁶ independently of one another are hydrogen, C₁-C₆-alkyl, C₂-C₆-alkenyl, C₂-C₆-alkynyl, C₃-C₈-cycloalkyl or C₄-C₈-cycloalkenyl;
   R^{43a} has one of the meanings given for R⁴¹ except for hydrogen;
   R⁴², R⁴⁸ and R⁵² may additionally be —CO—R⁴⁵,
   R⁴² may furthermore be —CO—OR⁴¹ or —CO—NR⁴³R^{43b}, where R^{43b} has one of the meanings given for R⁴¹,
   R⁴² and R⁴³ together may also form a C₃-C₆-alkylene group which may be interrupted by an oxygen atom or have a double bond;
   R⁴⁹ and R⁵⁰ together may also form a C₃-C₆-alkylene group which may be interrupted by an oxygen atom or have a double bond;
   R⁵⁰ may also be a radical or the formula A-CO—OR⁴¹ or —CO—NR⁴³R^{43b} in which A is C₁-C₄-alkylene;

R⁵¹ may also be a group of the formula NR⁴²R⁴³, N═CR⁴⁹R⁵⁰ or N═C(R⁴⁵)NR⁴²R⁴³;
where the aliphatic or alicyclic groups of the radical definitions of R⁴¹-R⁵⁶ for their part may be partially or fully halogenated and/or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy.
   and/or their agriculturally useful salts for controlling plant-damaging fungi.

The present invention furthermore provides a composition for controlling harmful fungi, which composition comprises at least one compound of the general formula I and/or one agriculturally acceptable salt thereof and at least one liquid or solid carrier.

The present invention furthermore provides novel 5-hetaryl-4-aminopyrimidines of the general formula I in which Het, R¹, R², R³ and R⁴ are as defined above, where at least one of the radicals R¹ and R² is different from hydrogen and where R³ is not hydrogen or C₁-C₈-alkyl if R⁴ is chlorine, NH₂ or methyl. The invention also provides salts of the 5-hetaryl-4-aminopyrimidines of the general formula I, in particular their agriculturally acceptable salts but also their pharmaceutically acceptable salts.

The present invention furthermore provides the use of 5-hetaryl-4-aminopyrimidines of the general formula I and/or of a pharmaceutically acceptable salt thereof as a pharmaceutical, in particular for the treatment of cancer.

The present invention furthermore provides pharmaceutical compositions, comprising at least one 5-hetaryl-4-aminopyrimidine of the general formula I and/or a pharmaceutically acceptable salt thereof and a pharmaceutically acceptable carrier.

The present invention furthermore provides the use of 5-hetaryl-4-aminopyrimidines of the general formula I and/or pharmaceutically acceptable salts thereof in the manufacture of a medicament for the treatment of cancer.

The present invention furthermore provides a method for cancer treatment in mammals, which comprises administering to the mammal in need thereof an effective amount of a 5-hetaryl-4-amino pyrimidine of the formula I and/or a pharmaceutically acceptable salt thereof.

Depending on the substitution pattern, the compounds of the formula I may have one or more centers of chirality, in which case they are present as mixtures of enantiomers or diastereomers. The invention provides both the pure enantiomers or diastereomers and their mixtures. Suitable compounds of the formula I also include all possible stereoisomers (cis/transisomers) and mixtures thereof.

Suitable agriculturally useful salts are especially the salts of those cations or the acid addition salts of those acids whose cations and anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium iron which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

Suitable pharmaceutically acceptable salts are especially physiologically tolerated salts of the compound I, especially acid addition salts with physiologically tolerated acids. Examples of suitable physiologically tolerated organic and inorganic acids are hydrochloric acid, hydrobromic acid, phosphoric acid, nitric acid, sulfuric acid, C₁-C₄-alkylsulfonic acids such as methanesulfonic acid, cycloaliphatic sulfonic acids such as S-(+)-10-camphorsulfonic acids, aromatic sulfonic acids such as benzenesulfonic acid, cis- and trans-cinnamic acid, fluoric acid and toluenesulfonic acid, C₂-C₁₀ hydroxycarboxylic acids such as glycolic acid, di- and tri-C₂-C₁₀ carboxylic acids and hydroxycarboxylic acids such as oxalic acid, malonic acid, maleic acid, fumaric acid, lactic acid, tartaric acid, adipic acid, citric acid, mucic acid and benzoic acid. Other suitable acids are described for example in Fortschritte der Arzneimittelforschung [Advances in Drug Research], Volume 10, pages 224 ff., Birkhäuser Verlag, Basel and Stuttgart, 1966, which is hereby incorporated in its entirety by way of reference. The physiologically tolerated salts of the compounds I may be present as the mono-, bis-, tris- and tetrakissalts, that is, they may contain 1, 2, 3 or 4 of the aforementioned acid molecules per molecule of formula I. The acid molecules may be present in their acidic form or as anions.

In the definitions of the variables given in the formulae above, collective terms are used which are generally representative for the substituents in question. The term Cₙ-Cₘ indicates the number of carbon atoms possible in each case in the substituent or substituent moiety in question:

halogen: fluorine, chlorine, bromine and iodine;
alkyl and the alkyl moieties in alkyloxy, alkylthio, alkylsulfinyl and alkylsulfonyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like;
haloalkyl: straight-chain or branched alkyl groups having 1 to 2, 4, 6 or 8 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluorethyl, 2,2-dichloro-2-fluoroethyl, 2,2,2-trichloroethyl, pentafluoroethyl or 1,1,1-trifluoroprop-2-yl;
alkenyl and the alkenyl moieties in alkenyloxy: monounsaturated straight-chain or branched hydrocarbon radicals having 2 to 4, 2 to 6, 2 to 8 or 2 to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;
alkadienyl: diunsaturated straight-chain or branched hydrocarbon radicals having 4 to carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;
haloalkenyl: unsaturated straight-chain or branched hydrocarbon radicals having 2 to 10 carbon atoms and a double bond in any position (as mentioned above), where some or all of the hydrogen atoms in these groups may be replaced by halogen atoms as mentioned above, in particular fluorine, chlorine and bromine;
alkynyl and the alkynyl moieties in alkynyloxy: straight-chain or branched hydrocarbon groups having 2 to 4, 2 to 6, 2 to 8 or 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;
cycloalkyl and the cycloalkyl moieties in cycloalkoxy: monocyclic saturated hydrocarbon groups having 3 to 8 carbon ring members, such as cyclopropyl, cyclobutyl, cyclopentyl, cyclohexyl, cycloheptyl and cyclooctyl;
cycloalkenyl: monocyclic monounsaturated hydrocarbon groups having 3 to 8, preferably 5 to 6, carbon ring members, such as cyclopenten-1-yl, cyclopenten-3-yl, cyclohexen-1-yl, cyclohexen-3-yl, cyclohexen-4-yl and the like;
bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like;
C₁-C₄-alkoxy: an alkyl group having 1 to 4 carbon atoms which is attached via an oxygen, for example, methoxy, ethoxy, n-propoxy, 1-methylethoxy, butoxy, 1-methylpropoxy, 2-methylpropoxy or 1,1-dimethylethoxy;
C₁-C₈-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;
C₁-C₈-haloalkoxy: C₁-C₄-haloalkoxy as mentioned above and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
alkenyloxy: alkenyl as mentioned above which is attached via an oxygen atom, for example C₃-C₆-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;
alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;
alkylthio: alkyl, as defined above which is attached via a sulfur atom;
alkylsulfinyl: alkyl as defined above which is attached via an SO group;
alkylsulfonyl: alkyl as defined above which is attached via an S(O)₂ group;
a 5-, 6-, 7-, 8-, 9- or 10-membered saturated, partially unsaturated or aromatic heterocycle which contains 1, 2, 3 or 4 heteroatoms from the group consisting of oxygen, nitrogen and sulfur:
a five- or six-membered saturated or partially unsaturated heterocycle (hereinbelow also referred to as heterocyclyl) which contains one, two, three or four heteroatoms from the group consisting of oxygen, nitrogen and sulfur as ring members: for example monocyclic saturated or partially unsaturated heterocycles which contain, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which contains one, two, three or four heteroatoms from the group consisting of oxygen, nitrogen and sulfur as ring members: for example mono- and bicyclic heterocycles having 7 ring members which contain, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra-. and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra-. and hexahydro-1,3-diazepinyl, tetra- and hexahydro-1,4-diazepinyl, tetra-. and hexahydro-1,3-oxazepinyl, tetra-. and hexahydro-1,4-oxazepinyl, tetra-. and hexahydro-1,3-dioxepinyl, tetra- and hexahydro-1,4-dioxepinyl and the corresponding -ylidene radicals;
a five- or six-membered aromatic heterocycle (=heteroaromatic radical, hetaryl) which contains one, two, three or four heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
alkylene: divalent unbranched chains of 1 to 6 CH₂ groups, for example CH₂, CH₂CH₂, CH₂CH₂CH₂, CH₂CH₂CH₂CH₂, CH₂CH₂CH₂CH₂CH₂ and CH₂CH₂CH₂CH₂CH₂CH₂;
oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
oxyalkylenoxy: divalent unbranched chains of 1 to 3 CH₂ groups, where both valences are attached to the skeleton via an oxygen atom, for example OCH₂O, OCH₂CH₂O and OCH₂CH₂CH₂O.

With a view to the fungicidal activity, preference is given to compounds of the general formula I in which at least one of the radicals R¹ or R² is different from hydrogen. From among these, preference is given to compounds of the general formula I in which R¹ is different from hydrogen and R² is hydrogen.

Preference is likewise given to compounds of the general formula I in which R¹ and R² are different from hydrogen and R² is C₁-C₄-alkyl, especially methyl or ethyl.

For the fungicidal activity of the compound I it is furthermore advantageous if the substituents Het, R¹, R², R³ and R⁴ and independently of one another and particularly preferably in combination have the meanings given below as being preferred:

Preference is given to compounds I in which Het carries at least one, for example 1, 2 or 3, substituents L. Preferred substituents L on Het are halogen, cyano, nitro, NH₂, C₁-C₆-alkylamino, di-C₁-C₆-alkylamino, C₁-C₆-alkyl, C₁-C₆-haloalkyl, C₁-C₆-alkoxy, C₁-C₆-alkylamino, di-C₁-C₆-alkylamino, NH—C(O)—C₁-C₆-alkyl, a group C(S)A² and a group C(O)A². Here, A² is as defined above and is preferably C₁-C₄-alkoxy, NH₂, C₁-C₄-alkylamino or di-C₁-C₄-alkylamino. Especially preferred radicals L independently of one another are chosen from the group consisting of fluorine, chlorine, bromine, cyano, nitro, C₁-C₄-alkyl, C₁-C₄-haloalkyl, C₁-C₄-alkoxy and C₁-C₄-alkoxycarbonyl, particularly preferably from the group consisting of fluorine, chlorine, C₁-C₂-alkyl, such as methyl or ethyl, C₁-C₂-fluoroalkyl, such as trifluoromethyl, C₁-C₂-alkoxy, such as methoxy, or C₁-C₂-alkoxycarbonyl, such as methoxycarbonyl.

Especially preferably at least one of the heteroatoms of the heteroaromatic radical Het and/or one substituent L is located in the ortho-position to the point of attachment of Het to the pyrimidine skeleton. Preferred substituents L in der ortho-position are fluorine, chlorine, bromine, C₁-C₂-alkyl, such as methyl or ethyl, C₁-C₂-fluoroalkyl, such as trifluoromethyl, and C₁-C₂-alkoxy, such as methoxy.

Especially preferably, preference is given to compounds of the formula I in which Het has at least one ring nitrogen atom. From among these, preference is given to those compounds of the formula I in which the ring nitrogen atom is located in the ortho-position to the point of attachment of Het to the 5-position of the pyrimidine skeleton.

Especially preferred are also compounds of the formula I in which Het has at least one ring sulfur atom. From among these, preference is given to those compounds of the formula I in which the ring sulfur atom is located in the ortho-position to the point of attachment of Het to the 5-position of the pyrimidine skeleton.

According to a first preferred embodiment of the invention, Het is a 5-membered heteroaromatic radical which has at least one nitrogen atom and optionally 1 or 2 further heteroatoms selected from the group consisting of O, S and N as ring members and which is unsubstituted or carries 1, 2 or 3 substituents L. Examples of these are compounds of the formula I in which Het is selected from the group consisting of pyrrolyl, pyrazolyl, imidazolyl, 1,2,3-triazolyl, 1,2,4-triazolyl, oxazolyl, thiazolyl, isoxazolyl and isothiazolyl, where Het is unsubstituted or carries 1, 2 or 3 substituents L.

From among the compounds I mentioned above, especial preference is given to those in which Het is thiazolyl, imidazolyl, pyrazolyl, 1,2,4-triazolyl or 1,2,3-triazolyl, where the radical mentioned above are unsubstituted or have 1, 2 or 3 substituents L. Especially preferred are those compounds I in which Het is pyrazol-1-yl which is unsubstituted or has 1, 2 or 3 substituents L. Especially preference is also given to those compounds I in which Het is thiazol-2-yl which is unsubstituted or has 1, 2 or 3 substituents L.

In this embodiment Het is in particular one of the radicals Het-1 to Het-31 listed below:

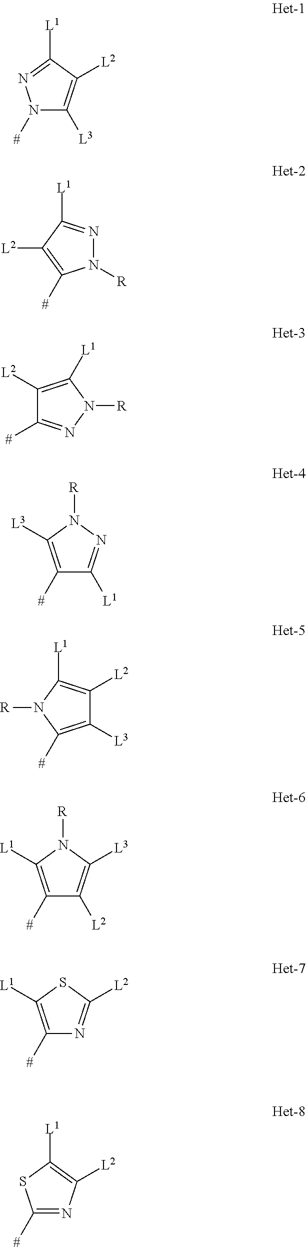

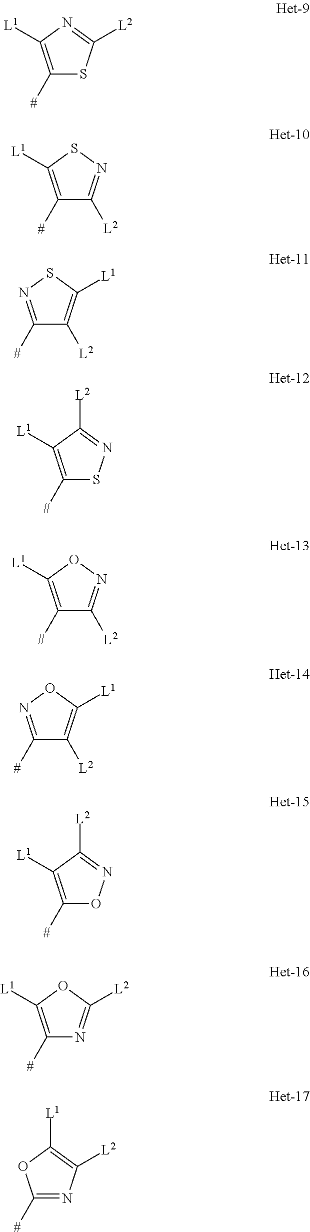

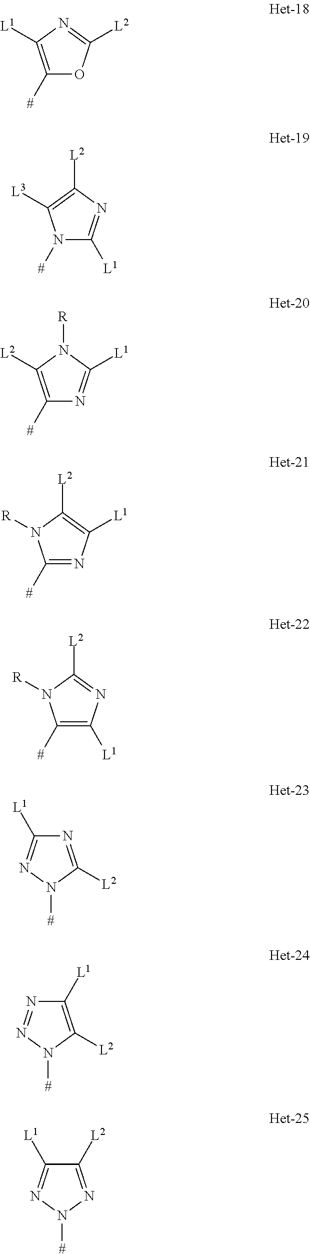

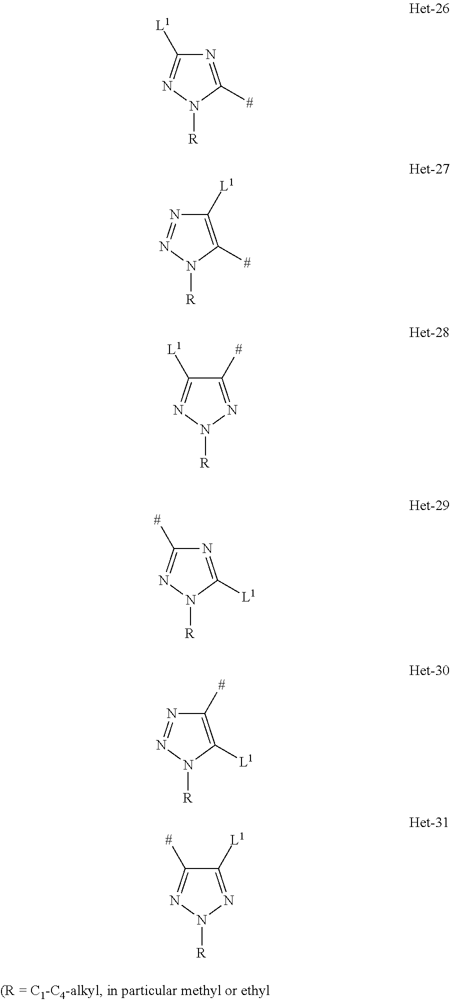

in which
#denotes the point of attachment to the 5-position of the pyrimidine ring; and
L¹, L², and L³ independently of one another are hydrogen or have one of the meanings mentioned for L.

The radicals L¹, L² and L³ independently of one another are preferably selected from the group consisting of hydrogen, halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl. In particularly preferred embodiments, L¹, L² and L³ independently of one another are selected from the group consisting of hydrogen, nitro, cyano, fluorine, chlorine, bromine, methyl, ethyl, isopropyl, trifluoromethyl, fluoromethyl, methoxy and methoxycarbonyl.

Examples of Het-1 are 3,5-dimethylpyrazol-1-yl, 3,5-diisopropylpyrazol-1-yl, 3-methyl-5-isopropylpyrazol-1-yl, 3-isopropyl-5-methylpyrazol-1-yl, 3-ethyl-5-methylpyrazol-1-yl, 3,4,5-trimethylpyrazol-1-yl, 3-chloropyrazol-1-yl, 3-methylpyrazol-1-yl, 3-methyl-4-chloropyrazol-1-yl, 3-trifluoromethylpyrazol-1-yl, 3-trifluoromethyl-5-methoxypyrazol-1-yl, 3-trifluoromethyl-5-methylpyrazol-1-yl, 3-methyl-5-methoxypyrazol-1-yl, 3,5-dichloro-4-methylpyrazol-1-yl, 3,5-dimethyl-4-chloropyrazol-1-yl, 3,5-ditrifluoromethylpyrazol-1-yl and 3,4-dichloro-5-trichloromethylpyrazole.

Examples of Het-2 are 1,3-dimethylpyrazol-5-yl and 1-methyl-3-trifluoromethylpyrazol-5-yl.

Examples of Het-3 are 1,5-dimethylpyrazol-3-yl and 1-methyl-5-methoxypyrazol-3-yl.

Examples of Het-4 include 1,3-dimethylpyrazol-4-yl, 1,5-dimethylpyrazol-4-yl, 1,3,5-trimethylpyrazol-4-yl, 1-methyl-3-trifluoromethylpyrazol-4-yl and 1-methyl-5-trifluoromethylpyrazol-4-yl.

Examples of Het-5 are 1-methylpyrrol-2-yl, 1,4-dimethylpyrrol-2-yl, 1-methyl-5-chloropyrrol-2-yl and 1-methyl-3,5-dichloropyrrol-2-yl.

Examples of Het-6 are 1,4-dimethylpyrazol-3-yl and 1-methylpyrazol-3-yl.

Examples of Het-7 are thiazol-4-yl 2-methylthiazol-4-yl, 2-methyl-5-bromothiazol-4-yl, 2-methyl-5-chlorothiazol-4-yl and 2,5-dichlorothiazol-4-yl.

An example of Het-8 is thiazol-2-yl.

An example of Het-9 is thiazol-5-yl.

Examples of Het-10 are 3-methylisothiazol-4-yl and 3-methyl-5-chloroisothiazol-4-yl.

An example of Het-11 is isothiazol-3-yl.

An example of Het-12 is isothiazol-5-yl.

Examples of Het-13 include isoxazol-4-yl 3,5-dimethylisoxazol-4-yl, 3-methylisoxazol-4-yl and 3-chloroisoxazol-4-yl.

An example of Het-14 is isoxazol-3-yl.

An example of Het-15 is isoxazol-5-yl.

Examples of Het-16 include oxazol-4-yl, 2-methyloxazol-4-yl and 2,5-dimethyloxazol-4-yl.

An example of Het-17 is oxazol-2-yl.

An example of Het-18 is oxazol-5-yl.

Examples of Het-19 include 4,5-dichloroimidazol-1-yl and 4,5-dimethylimidazol-1-yl.

An example of Het-20 is 1-methylimidazol-4-yl.

An example of Het-21 is 1-methylimidazol-2-yl.

An example of Het-22 is 1-methylimidazol-5-yl.

Examples of Het-23 include 3-chloro-1,2,4-triazol-1-yl, 3-fluoro-1,2,4-triazol-1-yl, 3-bromo-1,2,4-triazol-1-yl, 3-trifluoromethyl-1,2,4-triazol-1-yl, 3,5-dimethyl-1,2,4-triazol-1-yl, 3,5-dichloro-1,2,4-triazol-1-yl, 3,5-dibromo-1,2,4-triazol-1-yl, 3,5-difluoro-1,2,4-triazol-1-yl and 3,5-ditrifluoromethyl-1,2,4-triazol-1-yl.

Examples of Het-24 include 4,5-dimethyl-1,2,3-triazol-1-yl, 4,5-dichloro-1,2,3-triazol-1-yl, 4,5-dibromo-1,2,3-triazol-1-yl, 4,5-difluoro-1,2,3-triazol-1-yl, 4,5-ditrifluoromethyl-1,2,3-triazol-1-yl, 5-methyl-1,2,3-triazol-1-yl, 5-chloro-1,2,3-triazol-1-yl, 5-fluoro-1,2,3-triazol-1-yl, 5-bromo-1,2,3-triazol-1-yl, 5-trifluoromethyl-1,2,3-triazol-1-yl.

An example of Het-25 is 1,2,3-triazol-2-yl.

An example of Het-26 is 1-methyl-1,2,4-triazol-5-yl.

An example of Het-27 is 1-methyl-1,2,3-triazol-5-yl.

An example of Het-28 is 2-methyl-1,2,3-triazol-4-yl.

An example of Het-29 is 1-methyl-1,2,4-triazol-3-yl.

An example of Het-30 is 1-methyl-1,2,3-triazol-4-yl.

An example of Het-31 is 2-methyl-1,2,3-triazol-5-yl.

According to a further embodiment of the invention, Het is thienyl which is unsubstituted or has 1, 2 or 3 substituents L. Accordingly, Het is one of the radicals Het-32 or Het-33 below in which # denotes the point of attachment and L¹, L², and L³ independently of one another have the meanings given above for formulae Het-1 to Het-31.

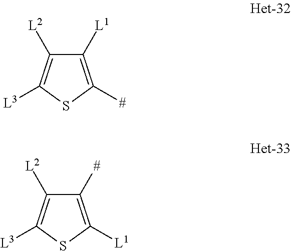

Examples of Het-32 are 2-thienyl, 5-methylthiophen-2-yl, 4-methylthiophen-2-yl, 5-chlorothiophen-2-yl, 3-cyanothiophen-2-yl, 5-acetylthiophen-2-yl, 5-bromothiophen-2-yl, 3,5-dichlorothiophen-2-yl, 3,4,5-trichlorothiophen-2-yl and 5-bromothiophen-2-yl.

Examples of Het-33 are 3-thienyl, 2-methylthiophen-3-yl, 2,5-dichlorothiophen-3-yl, 2,4,5-trichlorothiophen-3-yl and 2,5-dibromothiophen-3-yl.

According to a further embodiment of the invention, Het is furyl which is unsubstituted or has 1, 2 or 3 substituents L. Accordingly, Het is one of the radicals Het-32 or Het-33 below in which #denotes the point of attachment and L¹, L², and L³ independently of one another have the meanings given above for formulae Het-1 to Het-31.

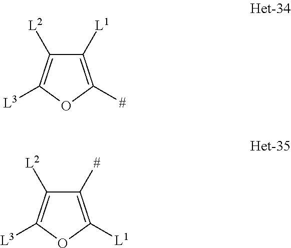

Examples of Het-34 are 2-furyl, 5-methylfuran-2-yl, 5-chlorofuran-2-yl, 4-methylfuran-2-yl, 3-cyanofuran-2-yl, 5-acetylfuran-2-yl, 5-bromofuran-2-yl, 3,5-dichlorofuran-2-yl, 3,4,5-trichlorofuran-2-yl and 5-bromofuran-2-yl.

Examples of Het-35 are 3-furyl, 2-methylfuran-3-yl, 2,5-dimethylfuran-3-yl and 2,5-dibromofuran-3-yl.

A further preferred embodiment of the invention relates to compounds of the general formula I in which Het is a 6-membered heteroaromatic radical which has 1, 2 or 3 nitrogen atoms as ring members and which is unsubstituted or carries 1, 2 or 3 substituents L. In this embodiment, Het is preferably pyridinyl, pyrimidinyl, pyrazinyl, pyridazinyl or triazinyl, in particular pyridinyl or pyrimidinyl which independently of one another are unsubstituted or carry 1, 2, 3 or 4 substituents L.

From among the compounds of this embodiment, preference is given to compounds of the general formula I in which Het is pyridinyl which optionally has 1, 2, 3 or 4 substituents L. From among these, particular preference is given to compounds of the formula I in which Het is 2-pyridinyl which has 1 or 2 substituents L. From among these, very particular preference is given to those compounds in which one of the substituents L is located in der 5-position of the pyridinyl ring. Moreover, from among these very particular preference is given to compounds I in which one of the substituents L is located in the 3-position of the pyridinyl ring. Here, L has in particular the meanings mentioned as being preferred.

From among the compounds of this embodiment, preference is furthermore given to compounds of the formula I in which Het is 3-pyridinyl which optionally has 1 or 2 substituents L. From among these, preference is given to those compounds which have a substituent L in the 2-position and/or a substituent L in the 4-position of the pyridine ring.

From among the compounds of this embodiment, preference is furthermore given to compounds of the formula I in which Het is 4-pyridinyl which optionally has 1 or 2 substituents L. From among these, preference is given to those compounds which have a substituent L in the 3-position and/or a substituent L in the 5-position of the pyridine ring.

From among the compounds of this embodiment, preference is furthermore given to compounds of the formula I in which Het is pyrimidinyl and in particular 2- or 4-pyrimidinyl which optionally has 1, 2 or 3 substituents L. From among these, particular preference is given to compounds of the formula I in which Het is 2-pyrimidinyl or 4-pyrimidinyl which has 1 or 2 substituents L. From among these, particular preference is given to those compounds in which one of the substituents L is located in the 5-position of the pyrimidinyl ring. Here, L has in particular the meanings mentioned as being preferred.

A further preferred embodiment of the invention relates to compounds of the formula I in which Het is 2-pyrazinyl which optionally has 1, 2 or 3 substituents L.

A further preferred embodiment of the invention relates to compounds of the formula I in which Het is 4-pyridazinyl which optionally has 1, 2 or 3 substituents L.

A further preferred embodiment of the invention relates to compounds of the formula I in which Het is 1,3,5-triazinyl which optionally has 1 or 2 substituents L.

Examples of particularly preferred heterocyclic radicals Het of this embodiment are the radicals Het-36 to Het-41 listed below:

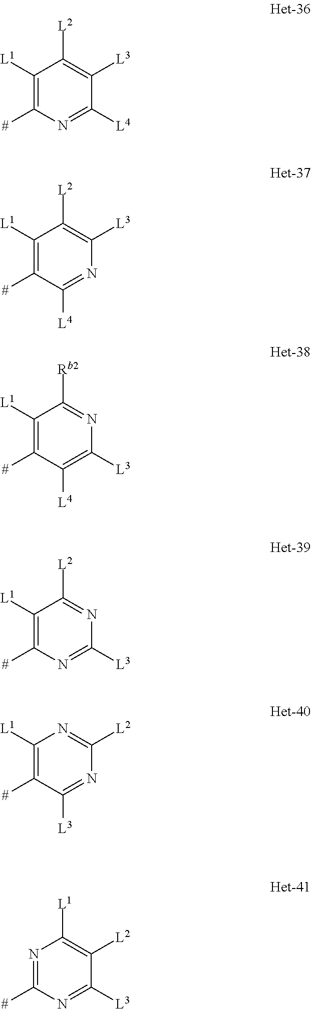

in which
#denotes the point of attachment; and
L¹, L², L³ and L⁴ independently of one another are hydrogen or have one of the meanings mentioned for L. Preferably, the radicals L¹, L², L³ and L⁴ independently of one another are selected from the group consisting of hydrogen, halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl. In particularly preferred embodiments, L¹, L², L³ and L⁴ independently of one another are selected from the group consisting of hydrogen, nitro, cyano, fluorine, chlorine, bromine, methyl, ethyl, isopropyl, trifluoromethyl, fluoromethyl, methoxy and methoxycarbonyl.

Examples of Het-36 are 2-pyridyl, 3-fluoropyridin-2-yl, 3-chloropyridin-2-yl, 3-bromo-2-pyridin-2-yl, 3-trifluoromethylpyridin-2-yl, 3-methylpyridin-2-yl, 3-ethylpyridin-2-yl, 3,5-difluoropyridin-2-yl, 3,5-dichloropyridin-2-yl, 3,5-dibromopyridin-2-yl, 3,5-dimethylpyridin-2-yl, 3-fluoro-5-trifluoromethylpyridin-2-yl, 3-chloro-5-fluoropyridin-2-yl, 3-chloro-5-methylpyridin-2-yl, 3-fluoro-5-chloropyridin-2-yl, 3-fluoro-5-methylpyridin-2-yl, 3-methyl-5-fluoropyridin-2-yl, 3-methyl-5-chloropyridin-2-yl, 5-nitropyridin-2-yl, 5-cyanopyridin-2-yl, 5-methoxycarbonylpyridin-2-yl, 5-trifluoromethylpyridin-2-yl, 5-methylpyridin-2-yl, 4-methylpyridin-2-yl and 6-methylpyridin-2-yl.

Examples of Het-37 are 3-pyridyl, 2-chloropyridin-3-yl, 2-bromopyridin-3-yl, 2-methylpyridin-3-yl, 2,4-dichloropyridin-3-yl, 2,4-dibromopyridin-3-yl, 2,4-difluoropyridin-3-yl, 2-fluoro-4-chloropyridin-3-yl, 2-chloro-4-fluoropyridin-3-yl, 2-chloro-4-methylpyridin-3-yl, 2-methyl-4-fluoropyridin-3-yl, 2-methyl-4-chloropyridin-3-yl, 2,4-dimethylpyridin-3-yl, 2,4,6-trichloropyridin-3-yl, 2,4,6-tribromopyridin-3-yl, 2,4,6-trimethylpyridin-3-yl and 2,4-dichloro-6-methylpyridin-3-yl.

Examples of Het-38 include 4-pyridyl, 3-chloropyridin-4-yl, 3-bromopyridin-4-yl, 3-methylpyridin-4-yl, 3,5-dichloropyridin-4-yl, 3,5-dibromopyridin-4-yl and 3,5-dimethylpyridin-4-yl.

Examples of Het-39 include 5-chloropyrimidin-4-yl, 5-fluoropyrimidin-4-yl, 5-fluoro-6-chloropyrimidin-4-yl, 2-methyl-6-trifluoromethylpyrimidin-4-yl, 2,5-dimethyl-6-trifluoromethylpyrimidin-4-yl, 5-methyl-6-trifluoromethylpyrimidin-4-yl, 6-trifluoromethylpyrimidin-4-yl, 2-methyl-5-fluoropyrimidin-4-yl, 2-methyl-5-chloropyrimidin-4-yl, 5-chloro-6-methylpyrimidin-4-yl, 5-chloro-6-ethylpyrimidin-4-yl, 5-chloro-6-isopropylpyrimidin-4-yl, 5-bromo-6-methylpyrimidin-4-yl, 5-fluoro-6-methylpyrimidin-4-yl, 5-fluoro-6-fluoromethylpyrimidin-4-yl, 2,6-dimethyl-5-chloropyrimidin-4-yl, 5,6-dimethylpyrimidin-4-yl, 2,5-dimethylpyrimidin-4-yl, 2,5,6-trimethylpyrimidin-4-yl and 5-methyl-6-methoxypyrimidin-4-yl.

Examples of Het-40 include 4-methylpyrimidin-5-yl, 4,6-dimethylpyrimidin-5-yl, 2,4,6-trimethylpyrimidin-5-yl and 4-trifluoromethyl-6-methylpyrimidin-5-yl.

Examples of Het-41 include 4,6-dimethylpyrimidin-2-yl, 4,5,6-trimethylpyrimidin-2-yl, 4,6-ditrifluoromethylpyrimidin-2-yl and 4,6-dimethyl-5-chloropyrimidin-2-yl.

Preferably, at least one of the radicals R¹ and R² is different from hydrogen.

R¹ is in particular C₁-C₈-alkyl, C₂-C₈-alkenyl, C₂-C₈-alkynyl, C₃-C₈-cycloalkyl, which may be mono-, di-, tri- or tetrasubstituted by halogen or C₁-C₄-alkyl, or C₁-C₈-haloalkyl.

From among these, a particularly preferred embodiment relates to compounds of the formula I in which R¹ is a group B:

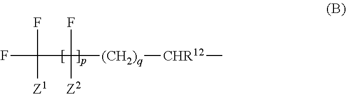

in which
p is 0 or 1;
q is 0 or 1;
Z¹ is hydrogen, fluorine or C₁-C₄-fluoroalkyl,
Z² is hydrogen or fluorine, or
Z¹ and Z² together, if p=1, form a double bond
R¹² is hydrogen or methyl.

Examples of such radicals B are 2,2,2-trifluoroethyl, 1-methyl-2,2,2-trifluoroethyl, 2,2,3,3,3-pentafluoropropyl, 3,3,4,4,4-pentafluorobutyl, 2,2,3,3,3-pentafluoro-1-methylpropyl and 2,3,3-trifluoro-2-propenyl.

From among these, a further preferred embodiment relates to compounds of the formula I in which R¹ is branched C₃-C₈-alkyl, such as 1-methylpropyl, 1-methylbutyl, 2-methylpropyl, 1,2-dimethylpropyl or 1,2,2-trimethylpropyl, or C₃-C₈-alkenyl, such as 2-propenyl, 2-methyl-2-propenyl.

From among these, a further preferred embodiment relates to compounds of the formula I in which R¹ is C₃-C₆-cycloalkyl which may be substituted by C₁-C₄-alkyl.

Here, R² is in particular hydrogen or C₁-C₄-alkyl, especially methyl or ethyl.

Preference is also given to compounds of the general formula I in which R¹ and R² together with the nitrogen atom to which they are attached are a saturated or monounsaturated, in particular 5- or 6-membered heterocyclic radical (heterocyclyl) as defined above which is attached via nitrogen. From among these, preference is given to those compounds of the formula I in which R¹ and R² together with the nitrogen atom to which they are attached form an optionally substituted piperidinyl, morpholinyl or thiomorpholinyl ring, especially a piperidinyl ring. Heterocyclyl is in particular unsubstituted or substituted by 1, 2 or 3 of the substituents mentioned above, preferred substituents on heterocyclyl being selected from the group consisting of halogen, C₁-C₄-alkyl and C₁-C₄-haloalkyl. From among these, particular preference is given to compound I in which R¹ and R² together with the nitrogen atom to which they are attached are from a 4-methylpiperidine ring, a 4-trifluoromethylpiperidine ring, a morpholine ring or a 3,4-dimethylpiperidine ring and especially a 4-methylpiperidine ring or a 3,4-dimethylpiperidine ring.

The invention furthermore particularly preferably provides compounds I in which R¹ and R² together with the nitrogen atom to which they are attached are a 5- or 6-membered heteroaromatic radical (heteroaryl) as defined above which is attached via nitrogen and which may be unsubstituted or substituted, preferably by 1, 2 or 3 of the substituents mentioned above. In this case, the group NR¹R² forms in particular a pyrazole ring which is attached via N and which is optionally substituted in the manner described above and especially by 1 or 2 of the following radicals: halogen, C₁-C₄-alkyl or C₁-C₄-haloalkyl, in particular by 2 methyl groups or 2 trifluoromethyl groups in the 3,5-position.

Very particular preference is given to compounds of the general formula I in which R¹ is selected from the group consisting of: CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl and cyclohexyl; and R² is hydrogen or methyl; and also to compounds I in which R¹ and R² together are —(CH₂)₂CH(CH₃)(CH₂)₂—, —(CH₂)₂CH(CF₃)(CH₂)₂— or —(CH₂)₂O(CH₂)₂—.

According to one embodiment of the invention, R³ is different from hydrogen. Preference is furthermore given to those compounds of the formula I in which R³ is halogen, cyano, C₁-C₄-alkyl, C₁-C₄-haloalkyl, C₁-C₄-alkoxy or C₁-C₂-haloalkoxy. Particularly preferred are compounds of the general formula I in which R³ is halogen, C₁-C₂-alkyl, cyano or C₁-C₂-alkoxy, such as chlorine, fluorine, bromine, methyl, cyano, methoxy or ethoxy. Particularly preferred are compounds I in which R³ is halogen and especially chlorine. Preference is also given to compounds I in which R³ is methoxy. Preference is also given to compounds I in which R³ is methyl. Preference is also given to compounds I in which R³ is cyano.

According to one embodiment of the invention, R⁴ is different from chlorine, OH, NH₂ or methyl, in particular from halogen, OH, NR⁴²R^{43a} or, C₁-C₈-alkyl. Preference according to the invention is given to compounds of the formula I in which R⁴ is selected from the group consisting of N₃, CN, C(═Z)OR⁴¹, C(═Z)NR⁴²R⁴³, C(═Z)NR⁴⁴—NR⁴²R⁴³, C(═Z)R⁴⁵, ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR⁵²(N═CR⁴⁹R⁵⁰), NR⁵²NR⁴²R⁴³, NR⁵²OR⁴¹ and C(═N—X—R⁴⁵)SR⁴¹.

In particularly preferred compounds of the formula I, R⁴ is selected from the group consisting of CN, C(═Z)OR⁴¹, C(═Z)NR⁴²R⁴³, C(═Z)NR⁴⁴—NR⁴²R⁴³, C(═Z)R⁴⁵ and C(═N—X—R⁴⁵) SR⁴¹.

From among these, particular preference is given to compound I in which R⁴ is one of the radicals below:

C(═O)OR⁴¹, such as C(═O)—C₁-C₄-alkyl,
C(═O)NR⁴²R⁴³, such as C(═O)NH₂ or C(═O)NH—C₁-C₄-alkyl,
C(═S)NR⁴²R⁴³, such as C(═S)NH₂,
C(═NOR⁵⁴)NR⁴²R⁴³, such as C(═N—O—C₁-C₄-alkyl)NH₂,
C(═O)NR⁴⁴—NR⁴²R⁴³, such as C(═O)NHNH₂,
C(═Z)R⁴⁵, such as C(═O)H, C(═O)—C₁-C₄-alkyl, C(═NO—C₁-C₄-alkyl)H, and
C(═NO—C₁-C₄-alkyl)-C₁-C₄-alkyl,
C(═N—OR⁴⁵)SR⁴¹ or
C(═N—R⁴⁵)SR⁴¹.

From among these, very particular preference is given to compounds I in which R⁴ is C(═O)NR⁴²R⁴³, especially C(═O)NH₂, or C(═NOR⁵⁴)NR⁴²R⁴³, particularly preferably C(═N—O—C₁-C₄-alkyl)NH₂, and especially C(═NOCH₃)NH₂.

Preference is also given to compounds of the formula I in which R⁴ is selected from the group consisting of ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR⁵²(N═CR⁴⁹R⁵⁰), NR⁵²NR⁴²R⁴³ and NR⁵²OR⁴¹.

From among these, particular preference is given to compound I in which R⁴ is one of the radicals below:

ON(═CR⁴⁹R⁵⁰), such as ON(═C(C₁-C₄-alkyl)₂),
NR⁵¹(C(═O)R⁴⁵), such as NH(C═O)H and NH(C(═O)—C₁-C₄-alkyl,
NR⁵¹(C(═O)OR⁴¹), such as NH(C(═O)O—C₁-C₄-alkyl,
NR⁵¹(C(═O)—NR⁴²R⁴³), such as NH(C(═O)NH₂ or NH(C(═O)NH C₁-C₄-alkyl,
NR⁵²(N═CR⁴⁹R⁵⁰), such as NH(N═C(CH₃)CH(CH₃)C(═O)OC₁-C₄-alkyl
NR⁵²OR⁴¹, such as N(C(═O)CH₃)(O—C₁-C₄-alkyl),

Examples of radicals NR⁵²NR⁴²R⁴³ are NHNHC(═O)OCH₃, NHNHC(═O)OC₂H₅, NHNHC(═O)OC₃H₇, NHNHC(═O)OC₄H₉.

Besides, R⁵ and R⁶ independently of one another are preferably hydrogen or C₁-C₄-alkyl.

R⁷ is preferably hydrogen or in particular C₁-C₆-alkyl.

R⁸ and R⁹ independently of one another are preferably hydrogen or C₁-C₆-alkyl.

R¹⁰ and R¹¹ independently of one another are preferably selected from the group consisting of hydrogen and C₁-C₆-alkyl.

Furthermore, A¹ is preferably hydrogen, C₁-C₆-alkyl or amino. The index n is preferably 0, 1 or 2.

A² is preferably C₁-C₄-alkoxy, NH₂, C₁-C₄-alkylamino or di-C₁-C₄-alkylamino.

Z is preferably O, S or NOR⁵⁴.

X is preferably a direct bond.

R⁴¹, R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵ and R⁵⁶ are preferably hydrogen or C₁-C₄-alkyl.

R⁴² is preferably hydrogen, C₁-C₄-alkyl, —CO—OR⁴¹ or —COR⁴⁵.

Especially preferred are the following groups of compounds of the formulae I.1 to I.11:

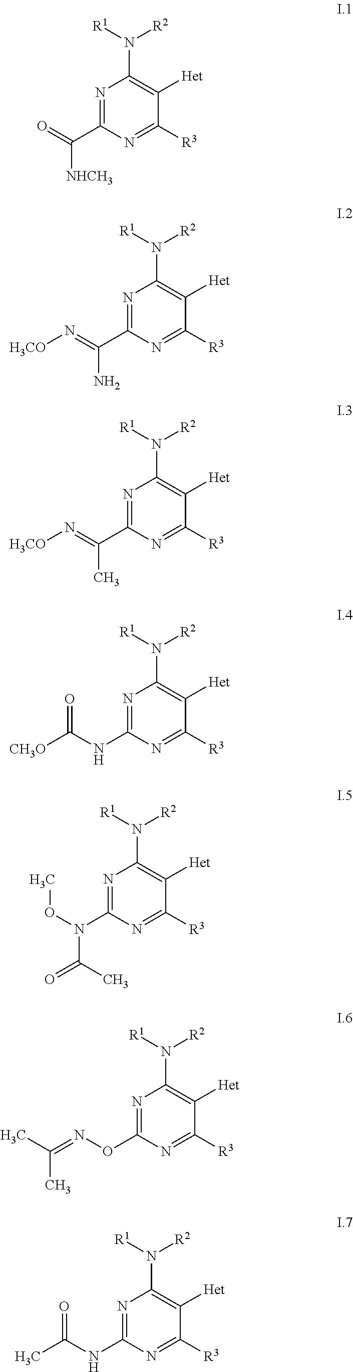

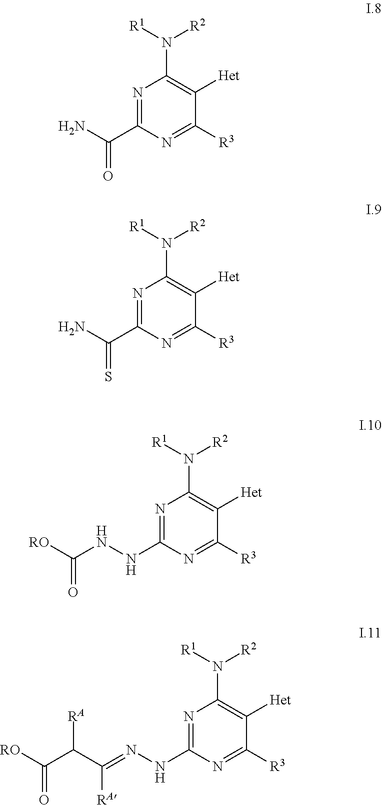

In the formulae I.1 to I.11, R¹, R², R³ and Het are as defined above and in particular as defined as being preferred above. In the formulae I.10 and I.11, R is C₁-C₄-alkyl, in particular methyl, and R^{A} and R^{A′} are C₁-C₄-alkyl, in particular methyl.

With a view to their use, the compounds I compiled in Tables 1 to 155 below are especially preferred. Moreover, groups mentioned for a substituent Het in Tables 1 to 155 are per se, independently of the combination in which they are mentioned, a particularly preferred embodiment of the substituent in question.

### Table 1

Compounds of the formulae I.1, I.2, I.3, I.4, I.5, I.6, I.7, I.8, I.9, I.10 and I.11 in which Het is 3-methyl-5-isopropylpyrazol-1-yl and the combination of R³, R¹ and R² for a compound corresponds in each case to one row of Table A.

### Table 2

Compounds of the formulae I.1, I.2, I.3, I.4, I.5, I.6, I.7, I.8, I.9, I.10 and I.11 in which Het is 3,5-dimethylpyrazol-1-yl and the combination of R³, R¹ and R² for a compound corresponds in each case to one row of Table A.

### Table 3

Compounds of the formulae I.1, I.2, I.3, I.4, I.5, I.6, I.7, I.8, I.9, I.10 and I.11 in which Het is 3-isopropyl-5-methylpyrazol-1-yl and the combination of R³, R¹ and R² for a compound corresponds in each case to one row of Table A.

### Table 4

Compounds of the formulae I.1, I.2, I.3, I.4, I.5, I.6, I.7, I.8, I.9, I.10 and I.11 in which Het is 3-ethyl-5-methylpyrazol-1-yl and the combination of R³, R¹ and R² for a compound corresponds in each case to one row of Table A.

### Table 5

Compounds of the formulae I.1, I.2, I.3, I.4, I.5, I.6, I.7, I.8, I.9, I.10 and I.11 in which Het is 3-methyl-5-methoxypyrazol-1-yl and the combination of R³, R¹ and R² for a compound corresponds in each case to one row of Table A.

### Table 6

Compounds of the formulae I.1, I.2, I.3, I.4, I.5, I.6, I.7, I.8, I.9, I.10 and I.11 in which Het is 3,4,5-trimethylpyrazol-1-yl and the combination of R³, R¹ and R² for a compound corresponds in each case to one row of Table A.

### Table 7

Compounds of the formulae I.1, I.2, I.3, I.4, I.5, I.6, I.7, I.8, I.9, I.10 and I.11 in which Het is 3,5-dimethyl-4-chloropyrazol-1-yl and the combination of R³, R¹ and R² for a compound corresponds in each case to one row of Table A.

### Table 8

Compounds of the formulae I.1, I.2, I.3, I.4, I.5, I.6, I.7, I.8, I.9, I.10 and I.11 in which Het is 3-chloropyrazol-1-yl and the combination of R³, R¹ and R² for a compound corresponds in each case to one row of Table A.

### Table 9

Compounds of the formulae I.1, I.2, I.3, I.4, I.5, I.6, I.7, I.8, I.9, I.10 and I.11 in which Het is 3,4-dichloro-5-trichloromethylpyrazol-1-yl and the combination of R³, R¹ and R² for a compound corresponds in each case to one row of Table A.

### Table 10

Compounds of the formulae I.1, I.2, I.3, I.4, I.5, I.6, I.7, I.8, I.9, I.10 and I.11 in which Het is 3-methylpyrazol-1-yl and the combination of R³, R¹ and R² for a compound corresponds in each case to one row of Table A.

### Table 11

Compounds of the formulae I.1, I.2, I.3, I.4, I.5, I.6, I.7, I.8, I.9, I.10 and I.11 in which Het is 3,5-dichloro-4-methylpyrazol-1-yl and the combination of R³, R¹ and R² for a compound corresponds in each case to one row of Table A.

### Table 12

Compounds of the formulae I.1, I.2, I.3, I.4, I.5, I.6, I.7, I.8, I.9, I.10 and I.11 in which Het is 3-methyl-4-chloropyrazol-1-yl and the combination of R³, R¹ and R² for a compound corresponds in each case to one row of Table A.

### Table 13

Compounds of the formulae I.1, I.2, I.3, I.4, I.5, I.6, I.7, I.8, I.9, I.10 and I.11 in which Het is 1,3-dimethylpyrazol-5-yl and the combination of R³, R¹ and R² for a compound corresponds in each case to one row of Table A.

### Table 14

Compounds of the formulae I.1, I.2, I.3, I.4, I.5, I.6, I.7, I.8, I.9, I.10 and I.11 in which Het is 1-methyl-3-trifluoromethylpyrazol-5-yl and the combination of R³, R¹ and R² for a compound corresponds in each case to one row of Table A.

### Table 15

Compounds of the formulae I.1, I.2, I.3, I.4, I.5, I.6, I.7, I.8, I.9, I.10 and I.11 in which Het is 1,5-dimethylpyrazol-3-yl and the combination of R³, R¹ and R² for a compound corresponds in each case to one row of Table A.

### Table 16

Compounds of the formulae I.1, I.2, I.3, I.4, I.5, I.6, I.7, I.8, I.9, I.10 and I.11 in which Het is 1-methyl-5-methoxypyrazol-3-yl and the combination of R³, R¹ and R² for a compound corresponds in each case to one row of Table A.

### Table 17

Compounds of the formulae I.1, I.2, I.3, I.4, I.5, I.6, I.7, I.8, I.9, I.10 and I.11 in which Het is 1,3,5-trimethylpyrazol-4-yl and the combination of R³, R¹ and R² for a compound corresponds in each case to one row of Table A.

### Table 18

Compounds of the formulae I.1, I.2, I.3, I.4, I.5, I.6, I.7, I.8, I.9, I.10 and I.11 in which Het is 1-methyl-3-trifluoromethylpyrazol-4-yl and the combination of R³, R¹ and R² for a compound corresponds in each case to one row of Table A.

### Table 19

Compounds of the formulae I.1, I.2, I.3, I.4, I.5, I.6, I.7, I.8, I.9, I.10 and I.11, hydrogen and Het 1,3-dimethylpyrazol-4-yl and the combination of R³, R¹ and R² for a compound corresponds in each case to one row of Table A.

### Table 20

Compounds of the formulae I.1, I.2, I.3, I.4, I.5, I.6, I.7, I.8, I.9, I.10 and I.11 in which Het is 1-methyl-5-trifluoromethylpyrazol-4-yl and the combination of R³, R¹ and R² for a compound corresponds in each case to one row of Table A.

### Table 21

Compounds of the formulae I.1, I.2, I.3, I.4, I.5, I.6, I.7, I.8, I.9, I.10 and I.11 in which Het is 1,5-dimethylpyrazol-4-yl and the combination of R³, R¹ and R² for a compound corresponds in each case to one row of Table A.

### Table 22

Compounds of the formulae I.1, I.2, I.3, I.4, I.5, I.6, I.7, I.8, I.9, I.10 and I.11 in which Het is 1-methylpyrrol-2-yl and the combination of R³, R¹ and R² for a compound corresponds in each case to one row of Table A.

### Table 23

Compounds of the formulae I.1, I.2, I.3, I.4, I.5, I.6, I.7, I.8, I.9, I.10 and I.11 in which Het is 1,4-dimethylpyrrol-2-yl and the combination of R³, R¹ and R² for a compound corresponds in each case to one row of Table A.

### Table 24

Compounds of the formulae I.1, I.2, I.3, I.4, I.5, I.6, I.7, I.8, I.9, I.10 and I.11 in which Het is 1-methyl-5-chloropyrrol-2-yl and the combination of R³, R¹ and R² for a compound corresponds in each case to one row of Table A.

### Table 25

Compounds of the formulae I.1, I.2, I.3, I.4, I.5, I.6, I.7, I.8, I.9, I.10 and I.11 in which Het is 1-methyl-3,5-dichloropyrrol-2-yl and the combination of R³, R¹ and R² for a compound corresponds in each case to one row of Table A.

### Table 26

Compounds of the formulae I.1, I.2, I.3, I.4, I.5, I.6, I.7, I.8, I.9, I.10 and I.11 in which Het is 2-methylthiazol-4-yl and the combination of R³, R¹ and R² for a compound corresponds in each case to one row of Table A.

### Table 27

Compounds of the formulae I.1, I.2, I.3, I.4, I.5, I.6, I.7, I.8, I.9, I.10 and I.11 in which Het is thiazol-4-yl and the combination of R³, R¹ and R² for a compound corresponds in each case to one row of Table A.

### Table 28

Compounds of the formulae I.1, I.2, I.3, I.4, I.5, I.6, I.7, I.8, I.9, I.10 and I.11 in which Het is 2-methyl-5-chlorothiazol-4-yl and the combination of R³, R¹ and R² for a compound corresponds in each case to one row of Table A.

### Table 29

Compounds of the formulae I.1, I.2, I.3, I.4, I.5, I.6, I.7, I.8, I.9, I.10 and I.11 in which Het is 2,5-dichlorothiazol-4-yl and the combination of R³, R¹ and R² for a compound corresponds in each case to one row of Table A.

### Table 30

Compounds of the formulae I.1, I.2, I.3, I.4, I.5, I.6, I.7, I.8, I.9, I.10 and I.11 in which Het is 2-methyl-5-bromothiazol-4-yl and the combination of R³, R¹ and R² for a compound corresponds in each case to one row of Table A.

### Table 31

Compounds of the formulae I.1, I.2, I.3, I.4, I.5, I.6, I.7, I.8, I.9, I.10 and I.11 in which Het is 3-methylisothiazol-4-yl and the combination of R³, R¹ and R² for a compound corresponds in each case to one row of Table A.

### Table 32

Compounds of the formulae I.1, I.2, I.3, I.4, I.5, I.6, I.7, I.8, I.9, I.10 and I.11 in which Het is 3-methyl-5-chloroisothiazol-4-yl and the combination of R³, R¹ and R² for a compound corresponds in each case to one row of Table A.

### Table 33

Compounds of the formulae I.1, I.2, I.3, I.4, I.5, I.6, I.7, I.8, I.9, I.10 and I.11 in which Het is isoxazol-4-yl and the combination of R³, R¹ and R² for a compound corresponds in each case to one row of Table A.

### Table 34

Compounds of the formulae I.1, I.2, I.3, I.4, I.5, I.6, I.7, I.8, I.9, I.10 and I.11 in which Het is 3,5-dimethylisoxazol-4-yl and the combination of R³, R¹ and R² for a compound corresponds in each case to one row of Table A.

### Table 35

Compounds of the formulae I.1, I.2, I.3, I.4, I.5, I.6, I.7, I.8, I.9, I.10 and I.11 in which Het is 3-chloroisoxazol-4-yl and the combination of R³, R¹ and R² for a compound corresponds in each case to one row of Table A.

### Table 36

Compounds of the formulae I.1, I.2, I.3, I.4, I.5, I.6, I.7, I.8, I.9, I.10 and I.11 in which Het is 3-methylisoxazol-4-yl and the combination of R³, R¹ and R² for a compound corresponds in each case to one row of Table A.

### Table 37

Compounds of the formulae I.1, I.2, I.3, I.4, I.5, I.6, I.7, I.8, I.9, I.10 and I.11 in which Het is oxazol-4-yl and the combination of R³, R¹ and R² for a compound corresponds in each case to one row of Table A.

### Table 38

Compounds of the formulae I.1, I.2, I.3, I.4, I.5, I.6, I.7, I.8, I.9, I.10 and I.11 in which Het is 2,5-dimethyloxazol-4-yl and the combination of R³, R¹ and R² for a compound corresponds in each case to one row of Table A.

### Table 39

Compounds of the formulae I.1, I.2, I.3, I.4, I.5, I.6, I.7, I.8, I.9, I.10 and I.11 in which Het is 2-methyloxazol-4-yl and the combination of R³, R¹ and R² for a compound corresponds in each case to one row of Table A.

### Table 40

Compounds of the formulae I.1, I.2, I.3, I.4, I.5, I.6, I.7, I.8, I.9, I.10 and I.11 in which Het is 4,5-dichloroimidazol-1-yl and the combination of R³, R¹ and R² for a compound corresponds in each case to one row of Table A.

### Table 41

Compounds of the formulae I.1, I.2, I.3, I.4, I.5, I.6, I.7, I.8, I.9, I.10 and I.11 in which Het is 4,5-dimethylimidazol-1-yl and the combination of R³, R¹ and R² for a compound corresponds in each case to one row of Table A.

### Table 42

Compounds of the formulae I.1, I.2, I.3, I.4, I.5, I.6, I.7, I.8, I.9, I.10 and I.11 in which Het is 3,5-dimethyl-1,2,4-triazol-1-yl and the combination of R³, R¹ and R² for a compound corresponds in each case to one row of Table A.

### Table 43

Compounds of the formulae I.1, I.2, I.3, I.4, I.5, I.6, I.7, I.8, I.9, I.10 and I.11 in which Het is 3,5-dichloro-1,2,4-triazol-1-yl and the combination of R³, R¹ and R² for a compound corresponds in each case to one row of Table A.

### Table 44

Compounds of the formulae I.1, I.2, I.3, I.4, I.5, I.6, I.7, I.8, I.9, I.10 and I.11 in which Het is 3,5-dibromo-1,2,4-triazol-1-yl and the combination of R³, R¹ and R² for a compound corresponds in each case to one row of Table A.

### Table 45

Compounds of the formulae I.1, I.2, I.3, I.4, I.5, I.6, I.7, I.8, I.9, I.10 and I.11 in which Het is 3,5-difluoro-1,2,4-triazol-1-yl and the combination of R³, R¹ and R² for a compound corresponds in each case to one row of Table A.

### Table 46

Compounds of the formulae I.1, I.2, I.3, I.4, I.5, I.6, I.7, I.8, I.9, I.10 and I.11 in which Het is 3,5-ditrifluoromethyl-1,2,4-triazol-1-yl and the combination of R³, R¹ and R² for a compound corresponds in each case to one row of Table A.

### Table 47

Compounds of the formulae I.1, I.2, I.3, I.4, I.5, I.6, I.7, I.8, I.9, I.10 and I.11 in which Het is 3-methyl-1,2,4-triazol-1-yl and the combination of R³, R¹ and R² for a compound corresponds in each case to one row of Table A.

### Table 48

Compounds of the formulae I.1, I.2, I.3, I.4, I.5, I.6, I.7, I.8, I.9, I.10 and I.11 in which Het is 3-chloro-1,2,4-triazol-1-yl and the combination of R³, R¹ and R² for a compound corresponds in each case to one row of Table A.

### Table 49

Compounds of the formulae I.1, I.2, I.3, I.4, I.5, I.6, I.7, I.8, I.9, I.10 and I.11 in which Het is 3-fluoro-1,2,4-triazol-1-yl and the combination of R³, R¹ and R² for a compound corresponds in each case to one row of Table A.

### Table 50

Compounds of the formulae I.1, I.2, I.3, I.4, I.5, I.6, I.7, I.8, I.9, I.10 and I.11 in which Het is 3-bromo-1,2,4-triazol-1-yl and the combination of R³, R¹ and R² for a compound corresponds in each case to one row of Table A.

### Table 51

Compounds of the formulae I.1, I.2, I.3, I.4, I.5, I.6, I.7, I.8, I.9, I.10 and I.11 in which Het is 3-trifluoromethyl-1,2,4-triazol-1-yl and the combination of R³, R¹ and R² for a compound corresponds in each case to one row of Table A.

### Table 52

Compounds of the formulae I.1, I.2, I.3, I.4, I.5, I.6, I.7, I.8, I.9, I.10 and I.11 in which Het is 4,5-dimethyl-1,2,3-triazol-1-yl and the combination of R³, R¹ and R² for a compound corresponds in each case to one row of Table A.

### Table 53

Compounds of the formulae I.1, I.2, I.3, I.4, I.5, I.6, I.7, I.8, I.9, I.10 and I.11 in which Het is 4,5-dichloro-1,2,3-triazol-1-yl and the combination of R³, R¹ and R² for a compound corresponds in each case to one row of Table A.

### Table 54

Compounds of the formulae I.1, I.2, I.3, I.4, I.5, I.6, I.7, I.8, I.9, I.10 and I.11 in which Het is 4,5-dibromo-1,2,3-triazol-1-yl and the combination of R³, R¹ and R² for a compound corresponds in each case to one row of Table A.

### Table 55

Compounds of the formulae I.1, I.2, I.3, I.4, I.5, I.6, I.7, I.8, I.9, I.10 and I.11 in which Het is 4,5-difluoro-1,2,3-triazol-1-yl and the combination of R³, R¹ and R² for a compound corresponds in each case to one row of Table A.

### Table 56

Compounds of the formulae I.1, I.2, I.3, I.4, I.5, I.6, I.7, I.8, I.9, I.10 and I.11 in which Het is 4,5-ditrifluoromethyl-1,2,3-triazol-1-yl and the combination of R³, R¹ and R² for a compound corresponds in each case to one row of Table A.

### Table 57

Compounds of the formulae I.1, I.2, I.3, I.4, I.5, I.6, I.7, I.8, I.9, I.10 and I.11 in which Het is 5-methyl-1,2,3-triazol-1-yl and the combination of R³, R¹ and R² for a compound corresponds in each case to one row of Table A.

### Table 58

Compounds of the formulae I.1, I.2, I.3, I.4, I.5, I.6, I.7, I.8, I.9, I.10 and I.11 in which Het is 5-chloro-1,2,3-triazol-1-yl and the combination of R³, R¹ and R² for a compound corresponds in each case to one row of Table A.

### Table 59

Compounds of the formulae I.1, I.2, I.3, I.4, I.5, I.6, I.7, I.8, I.9, I.10 and I.11 in which Het is 5-fluoro-1,2,3-triazol-1-yl and the combination of R³, R¹ and R² for a compound corresponds in each case to one row of Table A.

### Table 60

Compounds of the formulae I.1, I.2, I.3, I.4, I.5, I.6, I.7, I.8, I.9, I.10 and I.11 in which Het is 5-bromo-1,2,3-triazol-1-yl and the combination of R³, R¹ and R² for a compound corresponds in each case to one row of Table A.

### Table 61

Compounds of the formulae I.1, I.2, I.3, I.4, I.5, I.6, I.7, I.8, I.9, I.10 and I.11 in which Het is 5-trifluoromethyl-1,2,3-triazol-1-yl and the combination of R³, R¹ and R² for a compound corresponds in each case to one row of Table A.

### Table 62

Compounds of the formulae I.1, I.2, I.3, I.4, I.5, I.6, I.7, I.8, I.9, I.10 and I.11 in which Het is 2-thienyl and the combination of R³, R¹ and R² for a compound corresponds in each case to one row of Table A.

### Table 63

Compounds of the formulae I.1, I.2, I.3, I.4, I.5, I.6, I.7, I.8, I.9, I.10 and I.11 in which Het is 3,5-dichlorothiophen-2-yl and the combination of R³, R¹ and R² for a compound corresponds in each case to one row of Table A.

### Table 64

Compounds of the formulae I.1, I.2, I.3, I.4, I.5, I.6, I.7, I.8, I.9, I.10 and I.11 in which Het is 3,4,5-trichlorothiophen-2-yl and the combination of R³, R¹ and R² for a compound corresponds in each case to one row of Table A.

### Table 65

Compounds of the formulae I.1, I.2, I.3, I.4, I.5, I.6, I.7, I.8, I.9, I.10 and I.11 in which Het is 5-chlorothiophen-2-yl and the combination of R³, R¹ and R² for a compound corresponds in each case to one row of Table A.

### Table 66

Compounds of the formulae I.1, I.2, I.3, I.4, I.5, I.6, I.7, I.8, I.9, I.10 and I.11 in which Het is 5-bromothiophen-2-yl and the combination of R³, R¹ and R² for a compound corresponds in each case to one row of Table A.

### Table 67

Compounds of the formulae I.1, I.2, I.3, I.4, I.5, I.6, I.7, I.8, I.9, I.10 and I.11 in which Het is 5-methylthiophen-2-yl and the combination of R³, R¹ and R² for a compound corresponds in each case to one row of Table A.

### Table 68

Compounds of the formulae I.1, I.2, I.3, I.4, I.5, I.6, I.7, I.8, I.9, I.10 and I.11 in which Het is 2,5-dichlorothiophen-3-yl and the combination of R³, R¹ and R² for a compound corresponds in each case to one row of Table A.

### Table 69

Compounds of the formulae I.1, I.2, I.3, I.4, I.5, I.6, I.7, I.8, I.9, I.10 and I.11 in which Het is 2,5-dibromothiophen-3-yl and the combination of R³, R¹ and R² for a compound corresponds in each case to one row of Table A.

### Table 70

Compounds of the formulae I.1, I.2, I.3, I.4, I.5, I.6, I.7, I.8, I.9, I.10 and I.11 in which Het is 2-methylthiophen-3-yl and the combination of R³, R¹ and R² for a compound corresponds in each case to one row of Table A.

### Table 71

Compounds of the formulae I.1, I.2, I.3, I.4, I.5, I.6, I.7, I.8, I.9, I.10 and I.11 in which Het is 4-methylthiophen-2-yl and the combination of R³, R¹ and R² for a compound corresponds in each case to one row of Table A.

### Table 72

Compounds of the formulae I.1, I.2, I.3, I.4, I.5, I.6, I.7, I.8, I.9, I.10 and I.11 in which Het is 3-cyanothiophen-2-yl and the combination of R³, R¹ and R² for a compound corresponds in each case to one row of Table A.

### Table 73

Compounds of the formulae I.1, I.2, I.3, I.4, I.5, I.6, I.7, I.8, I.9, I.10 and I.11 in which Het is 5-acetylthiophen-2-yl and the combination of R³, R¹ and R² for a compound corresponds in each case to one row of Table A.

### Table 74

Compounds of the formulae I.1, I.2, I.3, I.4, I.5, I.6, I.7, I.8, I.9, I.10 and I.11 in which Het is 2-furyl and the combination of R³, R¹ and R² for a compound corresponds in each case to one row of Table A.

### Table 75

Compounds of the formulae I.1, I.2, I.3, I.4, I.5, I.6, I.7, I.8, I.9, I.10 and I.11 in which Het is 3-furyl and the combination of R³, R¹ and R² for a compound corresponds in each case to one row of Table A.

### Table 76

Compounds of the formulae I.1, I.2, I.3, I.4, I.5, I.6, I.7, I.8, I.9, I.10 and I.11 in which Het is 4-methylfuran-2-yl and the combination of R³, R¹ and R² for a compound corresponds in each case to one row of Table A.

### Table 77

Compounds of the formulae I.1, I.2, I.3, I.4, I.5, I.6, I.7, I.8, I.9, I.10 and I.11 in which Het is 3-cyanofuran-2-yl and the combination of R³, R¹ and R² for a compound corresponds in each case to one row of Table A.

### Table 78

Compounds of the formulae I.1, I.2, I.3, I.4, I.5, I.6, I.7, I.8, I.9, I.10 and I.11 in which Het is 5-acetylfuran-2-yl and the combination of R³, R¹ and R² for a compound corresponds in each case to one row of Table A.

### Table 79

Compounds of the formulae I.1, I.2, I.3, I.4, I.5, I.6, I.7, I.8, I.9, I.10 and I.11 in which Het is 3-chloropyridin-2-yl and the combination of R³, R¹ and R² for a compound corresponds in each case to one row of Table A.

### Table 80

Compounds of the formulae I.1, I.2, I.3, I.4, I.5, I.6, I.7, I.8, I.9, I.10 and I.11 in which Het is 3-bromopyridin-2-yl and the combination of R³, R¹ and R² for a compound corresponds in each case to one row of Table A.

### Table 81

Compounds of the formulae I.1, I.2, I.3, I.4, I.5, I.6, I.7, I.8, I.9, I.10 and I.11 in which Het is 3,5-dibromopyridin-2-yl and the combination of R³, R¹ and R² for a compound corresponds in each case to one row of Table A.

### Table 82

Compounds of the formulae I.1, I.2, I.3, I.4, I.5, I.6, I.7, I.8, I.9, I.10 and I.11 in which Het is 3,5-dimethylpyridin-2-yl and the combination of R³, R¹ and R² for a compound corresponds in each case to one row of Table A.

### Table 83

Compounds of the formulae I.1, I.2, I.3, I.4, I.5, I.6, I.7, I.8, I.9, I.10 and I.11 in which Het is 2-pyridyl and the combination of R³, R¹ and R² for a compound corresponds in each case to one row of Table A.

### Table 84

Compounds of the formulae I.1, I.2, I.3, I.4, I.5, I.6, I.7, I.8, I.9, I.10 and I.11 in which Het is 5-nitropyridin-2-yl and the combination of R³, R¹ and R² for a compound corresponds in each case to one row of Table A.

### Table 85

Compounds of the formulae I.1, I.2, I.3, I.4, I.5, I.6, I.7, I.8, I.9, I.10 and I.11 in which Het is 5-cyanopyridin-2-yl and the combination of R³, R¹ and R² for a compound corresponds in each case to one row of Table A.

### Table 86

Compounds of the formulae I.1, I.2, I.3, I.4, I.5, I.6, I.7, I.8, I.9, I.10 and I.11 in which Het is 5-methoxycarbonylpyridin-2-yl and the combination of R³, R¹ and R² for a compound corresponds in each case to one row of Table A.

### Table 87

Compounds of the formulae I.1, I.2, I.3, I.4, I.5, I.6, I.7, I.8, I.9, I.10 and I.11 in which Het is 5-methylpyridin-2-yl and the combination of R³, R¹ and R² for a compound corresponds in each case to one row of Table A.

### Table 88

Compounds of the formulae I.1, I.2, I.3, I.4, I.5, I.6, I.7, I.8, I.9, I.10 and I.11 in which Het is 4-methylpyridin-2-yl and the combination of R³, R¹ and R² for a compound corresponds in each case to one row of Table A.

### Table 89

Compounds of the formulae I.1, I.2, I.3, I.4, I.5, I.6, I.7, I.8, I.9, I.10 and I.11 in which Het is 3-methylpyridin-2-yl and the combination of R³, R¹ and R² for a compound corresponds in each case to one row of Table A.

### Table 90

Compounds of the formulae I.1, I.2, I.3, I.4, I.5, I.6, I.7, I.8, I.9, I.10 and I.11 in which Het is 3-ethylpyridin-2-yl and the combination of R³, R¹ and R² for a compound corresponds in each case to one row of Table A.

### Table 91

Compounds of the formulae I.1, I.2, I.3, I.4, I.5, I.6, I.7, I.8, I.9, I.10 and I.11 in which Het is 6-methylpyridin-2-yl and the combination of R³, R¹ and R² for a compound corresponds in each case to one row of Table A.

### Table 92

Compounds of the formulae I.1, I.2, I.3, I.4, I.5, I.6, I.7, I.8, I.9, I.10 and I.11 in which Het is 5-trifluoromethylpyridin-2-yl and the combination of R³, R¹ and R² for a compound corresponds in each case to one row of Table A.

### Table 93

Compounds of the formulae I.1, I.2, I.3, I.4, I.5, I.6, I.7, I.8, I.9, I.10 and I.11 in which Het is 3-trifluoromethylpyridin-2-yl and the combination of R³, R¹ and R² for a compound corresponds in each case to one row of Table A.

### Table 94

Compounds of the formulae I.1, I.2, I.3, I.4, I.5, I.6, I.7, I.8, I.9, I.10 and I.11 in which Het is 5-fluoropyridin-2-yl and the combination of R³, R¹ and R² for a compound corresponds in each case to one row of Table A.

### Table 95

Compounds of the formulae I.1, I.2, I.3, I.4, I.5, I.6, I.7, I.8, I.9, I.10 and I.11 in which Het is 3-fluoropyridin-2-yl and the combination of R³, R¹ and R² for a compound corresponds in each case to one row of Table A.

### Table 96

Compounds of the formulae I.1, I.2, I.3, I.4, I.5, I.6, I.7, I.8, I.9, I.10 and I.11 in which Het is 3,5-difluoropyridin-2-yl and the combination of R³, R¹ and R² for a compound corresponds in each case to one row of Table A.

### Table 97

Compounds of the formulae I.1, I.2, I.3, I.4, I.5, I.6, I.7, I.8, I.9, I.10 and I.11 in which Het is 3,5-dichloropyridin-2-yl and the combination of R³, R¹ and R² for a compound corresponds in each case to one row of Table A.

### Table 98

Compounds of the formulae I.1, I.2, I.3, I.4, I.5, I.6, I.7, I.8, I.9, I.10 and I.11 in which Het is 3-fluoro-5-methylpyridin-2-yl and the combination of R³, R¹ and R² for a compound corresponds in each case to one row of Table A.

### Table 99

Compounds of the formulae I.1, I.2, I.3, I.4, I.5, I.6, I.7, I.8, I.9, I.10 and I.11 in which Het is 3-fluoro-5-chloropyridin-2-yl and the combination of R³, R¹ and R² for a compound corresponds in each case to one row of Table A.

### Table 100

Compounds of the formulae I.1, I.2, I.3, I.4, I.5, I.6, I.7, I.8, I.9, I.10 and I.11 in which Het is 3-chloro-5-fluoropyridin-2-yl and the combination of R³, R¹ and R² for a compound corresponds in each case to one row of Table A.

### Table 101

Compounds of the formulae I.1, I.2, I.3, I.4, I.5, I.6, I.7, I.8, I.9, I.10 and I.11 in which Het is 3-chloro-5-methylpyridin-2-yl and the combination of R³, R¹ and R² for a compound corresponds in each case to one row of Table A.

### Table 102

Compounds of the formulae I.1, I.2, I.3, I.4, I.5, I.6, I.7, I.8, I.9, I.10 and I.11 in which Het is 3-methyl-5-chloropyridin-2-yl and the combination of R³, R¹ and R² for a compound corresponds in each case to one row of Table A.

### Table 103

Compounds of the formulae I.1, I.2, I.3, I.4, I.5, I.6, I.7, I.8, I.9, I.10 and I.11 in which Het is 3-methyl-5-fluoropyridin-2-yl and the combination of R³, R¹ and R² for a compound corresponds in each case to one row of Table A

### Table 104

Compounds of the formulae I.1, I.2, I.3, I.4, I.5, I.6, I.7, I.8, I.9, I.10 and I.11 in which Het is pyridin-3-yl and the combination of R³, R¹ and R² for a compound corresponds in each case to one row of Table A.

### Table 105

Compounds of the formulae I.1, I.2, I.3, I.4, I.5, I.6, I.7, I.8, I.9, I.10 and I.11 in which Het is 2-chloropyridin-3-yl and the combination of R³, R¹ and R² for a compound corresponds in each case to one row of Table A.

### Table 106

Compounds of the formulae I.1, I.2, I.3, I.4, I.5, I.6, I.7, I.8, I.9, I.10 and I.11 in which Het is 2,4-dichloropyridin-3-yl and the combination of R³, R¹ and R² for a compound corresponds in each case to one row of Table A.

### Table 107

Compounds of the formulae I.1, I.2, I.3, I.4, I.5, I.6, I.7, I.8, I.9, I.10 and I.11 in which Het is 2,4,6-trichloropyridin-3-yl and the combination of R³, R¹ and R² for a compound corresponds in each case to one row of Table A.

### Table 108

Compounds of the formulae I.1, I.2, I.3, I.4, I.5, I.6, I.7, I.8, I.9, I.10 and I.11 in which Het is 2-bromopyridin-3-yl and the combination of R³, R¹ and R² for a compound corresponds in each case to one row of Table A.

### Table 109

Compounds of the formulae I.1, I.2, I.3, I.4, I.5, I.6, I.7, I.8, I.9, I.10 and I.11 in which Het is 2,4-dibromopyridin-3-yl and the combination of R³, R¹ and R² for a compound corresponds in each case to one row of Table A.

### Table 110

Compounds of the formulae I.1, I.2, I.3, I.4, I.5, I.6, I.7, I.8, I.9, I.10 and I.11 in which Het is 2,4,6-tribromopyridin-3-yl and the combination of R³, R¹ and R² for a compound corresponds in each case to one row of Table A.

### Table 111

Compounds of the formulae I.1, I.2, I.3, I.4, I.5, I.6, I.7, I.8, I.9, I.10 and I.11 in which Het is 2-methylpyridin-3-yl and the combination of R³, R¹ and R² for a compound corresponds in each case to one row of Table A.

### Table 112

Compounds of the formulae I.1, I.2, I.3, I.4, I.5, I.6, I.7, I.8, I.9, I.10 and I.11 in which Het is 2,4-dimethylpyridin-3-yl and the combination of R³, R¹ and R² for a compound corresponds in each case to one row of Table A.

### Table 113

Compounds of the formulae I.1, I.2, I.3, I.4, I.5, I.6, I.7, I.8, I.9, I.10 and I.11 in which Het is 2,4,6-trimethylpyridin-3-yl and the combination of R³, R¹ and R² for a compound corresponds in each case to one row of Table A.

### Table 114

Compounds of the formulae I.1, I.2, I.3, I.4, I.5, I.6, I.7, I.8, I.9, I.10 and I.11 in which Het is 2,4-dichloro-6-methylpyridin-3-yl and the combination of R³, R¹ and R² for a compound corresponds in each case to one row of Table A.

### Table 115

Compounds of the formulae I.1, I.2, I.3, I.4, I.5, I.6, I.7, I.8, I.9, I.10 and I.11 in which Het is 2,4-difluoropyridin-3-yl and the combination of R³, R¹ and R² for a compound corresponds in each case to one row of Table A.

### Table 116

Compounds of the formulae I.1, I.2, I.3, I.4, I.5, I.6, I.7, I.8, I.9, I.10 and I.11 in which Het is 2-fluoro-4-chloropyridin-3-yl and the combination of R³, R¹ and R² for a compound corresponds in each case to one row of Table A.

### Table 117

Compounds of the formulae I.1, I.2, I.3, I.4, I.5, I.6, I.7, I.8, I.9, I.10 and I.11 in which Het is 2-chloro-4-fluoropyridin-3-yl and the combination of R³, R¹ and R² for a compound corresponds in each case to one row of Table A.

### Table 118

Compounds of the formulae I.1, I.2, I.3, I.4, I.5, I.6, I.7, I.8, I.9, I.10 and I.11 in which Het is 2-chloro-4-methylpyridin-3-yl and the combination of R³, R¹ and R² for a compound corresponds in each case to one row of Table A.

### Table 119

Compounds of the formulae I.1, I.2, I.3, I.4, I.5, I.6, I.7, I.8, I.9, I.10 and I.11 in which Het is 2-methyl-4-chloropyridin-3-yl and the combination of R³, R¹ and R² for a compound corresponds in each case to one row of Table A.

### Table 120

Compounds of the formulae I.1, I.2, I.3, I.4, I.5, I.6, I.7, I.8, I.9, I.10 and I.11 in which Het is 2-methyl-4-fluoropyridin-3-yl and the combination of R³, R¹ and R² for a compound corresponds in each case to one row of Table A.

### Table 121

Compounds of the formulae I.1, I.2, I.3, I.4, I.5, I.6, I.7, I.8, I.9, I.10 and I.11 in which Het is pyridin-4-yl and the combination of R³, R¹ and R² for a compound corresponds in each case to one row of Table A.

### Table 122

Compounds of the formulae I.1, I.2, I.3, I.4, I.5, I.6, I.7, I.8, I.9, I.10 and I.11 in which Het is 3-chloropyridin-4-yl and the combination of R³, R¹ and R² for a compound corresponds in each case to one row of Table A.

### Table 123

Compounds of the formulae I.1, I.2, I.3, I.4, I.5, I.6, I.7, I.8, I.9, I.10 and I.11 in which Het is 3,5-dichloropyridin-4-yl and the combination of R³, R¹ and R² for a compound corresponds in each case to one row of Table A.

### Table 124

Compounds of the formulae I.1, I.2, I.3, I.4, I.5, I.6, I.7, I.8, I.9, I.10 and I.11 in which Het is 3-bromopyridin-4-yl and the combination of R³, R¹ and R² for a compound corresponds in each case to one row of Table A.

### Table 125

Compounds of the formulae I.1, I.2, I.3, I.4, I.5, I.6, I.7, I.8, I.9, I.10 and I.11 in which Het is 3,5-dibromopyridin-4-yl and the combination of R³, R¹ and R² for a compound corresponds in each case to one row of Table A.

### Table 126

Compounds of the formulae I.1, I.2, I.3, I.4, I.5, I.6, I.7, I.8, I.9, I.10 and I.11 in which Het is 3-methylpyridin-4-yl and the combination of R³, R¹ and R² for a compound corresponds in each case to one row of Table A.

### Table 127

Compounds of the formulae I.1, I.2, I.3, I.4, I.5, I.6, I.7, I.8, I.9, I.10 and I.11 in which Het is 3,5-dimethylpyridin-4-yl and the combination of R³, R¹ and R² for a compound corresponds in each case to one row of Table A.

### Table 128

Compounds of the formulae I.1, I.2, I.3, I.4, I.5, I.6, I.7, I.8, I.9, I.10 and I.11 in which Het is 5-chloropyrimidin-4-yl and the combination of R³, R¹ and R² for a compound corresponds in each case to one row of Table A.

### Table 129

Compounds of the formulae I.1, I.2, I.3, I.4, I.5, I.6, I.7, I.8, I.9, I.10 and I.11 in which Het is 5-fluoropyrimidin-4-yl and the combination of R³, R¹ and R² for a compound corresponds in each case to one row of Table A.

### Table 130

Compounds of the formulae I.1, I.2, I.3, I.4, I.5, I.6, I.7, I.8, I.9, I.10 and I.11 in which Het is 2-methyl-6-trifluoromethylpyrimidin-4-yl and the combination of R³, R¹ and R² for a compound corresponds in each case to one row of Table A.

### Table 131

Compounds of the formulae I.1, I.2, I.3, I.4, I.5, I.6, I.7, I.8, I.9, I.10 and I.11 in which Het is 2,5-dimethyl-6-trifluoromethylpyrimidin-4-yl and the combination of R³, R¹ and R² for a compound corresponds in each case to one row of Table A.

### Table 132

Compounds of the formulae I.1, I.2, I.3, I.4, I.5, I.6, I.7, I.8, I.9, I.10 and I.11 in which Het is 5-methyl-6-trifluoromethylpyrimidin-4-yl and the combination of R³, R¹ and R² for a compound corresponds in each case to one row of Table A.

### Table 133

Compounds of the formulae I.1, I.2, I.3, I.4, I.5, I.6, I.7, I.8, I.9, I.10 and I.11 in which Het is 6-trifluoromethylpyrimidin-4-yl and the combination of R³, R¹ and R² for a compound corresponds in each case to one row of Table A.

### Table 134

Compounds of the formulae I.1, I.2, I.3, I.4, I.5, I.6, I.7, I.8, I.9, I.10 and I.11 in which Het is 5-chloro-6-ethylpyrimidin-4-yl and the combination of R³, R¹ and R² for a compound corresponds in each case to one row of Table A.

### Table 135

Compounds of the formulae I.1, I.2, I.3, I.4, I.5, I.6, I.7, I.8, I.9, I.10 and I.11 in which Het is 5-chloro-6-methylpyrimidin-4-yl and the combination of R³, R¹ and R² for a compound corresponds in each case to one row of Table A.

### Table 136

Compounds of the formulae I.1, I.2, I.3, I.4, I.5, I.6, I.7, I.8, I.9, I.10 and I.11 in which Het is 5-chloro-6-isopropylpyrimidin-4-yl and the combination of R³, R¹ and R² for a compound corresponds in each case to one row of Table A.

### Table 137

Compounds of the formulae I.1, I.2, I.3, I.4, I.5, I.6, I.7, I.8, I.9, I.10 and I.11 in which Het is 5-fluoro-6-chloropyrimidin-4-yl and the combination of R³, R¹ and R² for a compound corresponds in each case to one row of Table A.

### Table 138

Compounds of the formulae I.1, I.2, I.3, I.4, I.5, I.6, I.7, I.8, I.9, I.10 and I.11 in which Het is 5-bromo-6-methylpyrimidin-4-yl and the combination of R³, R¹ and R² for a compound corresponds in each case to one row of Table A.

### Table 139

Compounds of the formulae I.1, I.2, I.3, I.4, I.5, I.6, I.7, I.8, I.9, I.10 and I.11 in which Het is 5-fluoro-6-methylpyrimidin-4-yl and the combination of R³, R¹ and R² for a compound corresponds in each case to one row of Table A.

### Table 140

Compounds of the formulae I.1, I.2, I.3, I.4, I.5, I.6, I.7, I.8, I.9, I.10 and I.11 in which Het is 5-fluoro-6-fluoromethylpyrimidin-4-yl and the combination of R³, R¹ and R² for a compound corresponds in each case to one row of Table A.

### Table 141

Compounds of the formulae I.1, I.2, I.3, I.4, I.5, I.6, I.7, I.8, I.9, I.10 and I.11 in which Het is 2,6-dimethyl-5-chloropyrimidin-4-yl and the combination of R³, R¹ and R² for a compound corresponds in each case to one row of Table A.

### Table 142

Compounds of the formulae I.1, I.2, I.3, I.4, I.5, I.6, I.7, I.8, I.9, I.10 and I.11 in which Het is 5,6-dimethylpyrimidin-4-yl and the combination of R³, R¹ and R² for a compound corresponds in each case to one row of Table A.

### Table 143

Compounds of the formulae I.1, I.2, I.3, I.4, I.5, I.6, I.7, I.8, I.9, I.10 and I.11 in which Het is 2,5-dimethylpyrimidin-4-yl and the combination of R³, R¹ and R² for a compound corresponds in each case to one row of Table A.

### Table 144

Compounds of the formulae I.1, I.2, I.3, I.4, I.5, I.6, I.7, I.8, I.9, I.10 and I.11 in which Het is 2,5,6-trimethylpyrimidin-4-yl and the combination of R³, R¹ and R² for a compound corresponds in each case to one row of Table A.

### Table 145

Compounds of the formulae I.1, I.2, I.3, I.4, I.5, I.6, I.7, I.8, I.9, I.10 and I.11 in which Het is 5-methyl-6-methoxypyrimidin-4-yl and the combination of R³, R¹ and R² for a compound corresponds in each case to one row of Table A.

### Table 146

Compounds of the formulae I.1, I.2, I.3, I.4, I.5, I.6, I.7, I.8, I.9, I.10 and I.11 in which Het is 2-methyl-5-chloropyrimidin-4-yl and the combination of R³, R¹ and R² for a compound corresponds in each case to one row of Table A.

### Table 147

Compounds of the formulae I.1, I.2, I.3, I.4, I.5, I.6, I.7, I.8, I.9, I.10 and I.11 in which Het is 2-methyl-5-fluoropyrimidin-4-yl and the combination of R³, R¹ and R² for a compound corresponds in each case to one row of Table A.

### Table 148

Compounds of the formulae I.1, I.2, I.3, I.4, I.5, I.6, I.7, I.8, I.9, I.10 and I.11 in which Het is 4-methylpyrimidin-5-yl and the combination of R³, R¹ and R² for a compound corresponds in each case to one row of Table A.

### Table 149

Compounds of the formulae I.1, I.2, I.3, I.4, I.5, I.6, I.7, I.8, I.9, I.10 and I.11 in which Het is 4,6-dimethylpyrimidin-5-yl and the combination of R³, R¹ and R² for a compound corresponds in each case to one row of Table A.

### Table 150

Compounds of the formulae I.1, I.2, I.3, I.4, I.5, I.6, I.7, I.8, I.9, I.10 and I.11 in which Het is 4-trifluoromethyl-6-methylpyrimidin-5-yl and the combination of R³, R¹ and R² for a compound corresponds in each case to one row of Table A.

### Table 151

Compounds of the formulae I.1, I.2, I.3, I.4, I.5, I.6, I.7, I.8, I.9, I.10 and I.11 in which Het is 2,4,6-trimethylpyrimidin-5-yl and the combination of R³, R¹ and R² for a compound corresponds in each case to one row of Table A.

### Table 152

Compounds of the formulae I.1, I.2, I.3, I.4, I.5, I.6, I.7, I.8, I.9, I.10 and I.11 in which Het is 4,6-dimethylpyrimidin-2-yl and the combination of R³, R¹ and R² for a compound corresponds in each case to one row of Table A.

### Table 153

Compounds of the formulae I.1, I.2, I.3, I.4, I.5, I.6, I.7, I.8, I.9, I.10 and I.11 in which Het is 4,5,6-trimethylpyrimidin-2-yl and the combination of R³, R¹ and R² for a compound corresponds in each case to one row of Table A.

### Table 154

Compounds of the formulae I.1, I.2, I.3, I.4, I.5, I.6, I.7, I.8, I.9, I.10 and I.11 in which Het is 4,6-ditrifluoromethylpyrimidin-2-yl and the combination of R³, R¹ and R² for a compound corresponds in each case to one row of Table A.

### Table 155

Compounds of the formulae I.1, I.2, I.3, I.4, I.5, I.6, I.7, I.8, I.9, I.10 and I.11 in which Het is 4,6-dimethyl-5-chloropyrimidin-2-yl and the combination of R³, R¹ and R² for a compound corresponds in each case to one row of Table A.

TABLE A

| No. | R¹ | R² | R³ |
|---|---|---|---|
| A-1 | H | H | Cl |
| A-2 | CH₃ | H | Cl |
| A-3 | CH₃ | CH₃ | Cl |
| A-4 | CH₂CH₃ | H | Cl |
| A-5 | CH₂CH₃ | CH₃ | Cl |
| A-6 | CH₂CH₃ | CH₂CH₃ | Cl |
| A-7 | CH₂CF₃ | H | Cl |
| A-8 | CH₂CF₃ | CH₃ | Cl |
| A-9 | CH₂CF₃ | CH₂CH₃ | Cl |
| A-10 | CH₂CCl₃ | H | Cl |
| A-11 | CH₂CCl₃ | CH₃ | Cl |
| A-12 | CH₂CCl₃ | CH₂CH₃ | Cl |
| A-13 | CH₂CH₂CH₃ | H | Cl |
| A-14 | CH₂CH₂CH₃ | CH₃ | Cl |
| A-15 | CH₂CH₂CH₃ | CH₂CH₃ | Cl |
| A-16 | CH₂CH₂CH₃ | CH₂CH₂CH₃ | Cl |
| A-17 | CH(CH₃)₂ | H | Cl |
| A-18 | CH(CH₃)₂ | CH₃ | Cl |
| A-19 | CH(CH₃)₂ | CH₂CH₃ | Cl |
| A-20 | CH₂CH(CH₃)₂ | H | Cl |
| A-21 | CH₂CH(CH₃)₂ | CH₃ | Cl |
| A-22 | CH₂CH(CH₃)₂ | CH₂CH₃ | Cl |
| A-23 | CH₂CH(CH₃)₂ | CH₂CH₂CH₃ | Cl |
| A-24 | CH₂CH₂CH₂CH₃ | H | Cl |
| A-25 | CH₂CH₂CH₂CH₃ | CH₃ | Cl |
| A-26 | CH₂CH₂CH₂CH₃ | CH₂CH₃ | Cl |
| A-27 | CH₂CH₂CH₂CH₃ | CH₂CH₂CH₃ | Cl |
| A-28 | CH₂CH₂CH₂CH₃ | CH₂CH₂CH₂CH₃ | Cl |
| A-29 | (±) CH(CH₃)—CH₂CH₃ | H | Cl |
| A-30 | (±) CH(CH₃)—CH₂CH₃ | CH₃ | Cl |
| A-31 | (±) CH(CH₃)—CH₂CH₃ | CH₂CH₃ | Cl |
| A-32 | (S) CH(CH₃)—CH₂CH₃ | H | Cl |
| A-33 | (S) CH(CH₃)—CH₂CH₃ | CH₃ | Cl |
| A-34 | (S) CH(CH₃)—CH₂CH₃ | CH₂CH₃ | Cl |
| A-35 | (R) CH(CH₃)—CH₂CH₃ | H | Cl |
| A-36 | (R) CH(CH₃)—CH₂CH₃ | CH₃ | Cl |
| A-37 | (R) CH(CH₃)—CH₂CH₃ | CH₂CH₃ | Cl |
| A-38 | (±) CH(CH₃)—CH(CH₃)₂ | H | Cl |
| A-39 | (±) CH(CH₃)—CH(CH₃)₂ | CH₃ | Cl |
| A-40 | (±) CH(CH₃)—CH(CH₃)₂ | CH₂CH₃ | Cl |
| A-41 | (S) CH(CH₃)—CH(CH₃)₂ | H | Cl |
| A-42 | (S) CH(CH₃)—CH(CH₃)₂ | CH₃ | Cl |
| A-43 | (S) CH(CH₃)—CH(CH₃)₂ | CH₂CH₃ | Cl |
| A-44 | (R) CH(CH₃)—CH(CH₃)₂ | H | Cl |
| A-45 | (R) CH(CH₃)—CH(CH₃)₂ | CH₃ | Cl |
| A-46 | (R) CH(CH₃)—CH(CH₃)₂ | CH₂CH₃ | Cl |
| A-47 | (±) CH(CH₃)—C(CH₃)₃ | H | Cl |
| A-48 | (±) CH(CH₃)—C(CH₃)₃ | CH₃ | Cl |
| A-49 | (±) CH(CH₃)—C(CH₃)₃ | CH₂CH₃ | Cl |
| A-50 | (S) CH(CH₃)—C(CH₃)₃ | H | Cl |
| A-51 | (S) CH(CH₃)—C(CH₃)₃ | CH₃ | Cl |
| A-52 | (S) CH(CH₃)—C(CH₃)₃ | CH₂CH₃ | Cl |
| A-53 | (R) CH(CH₃)—C(CH₃)₃ | H | Cl |
| A-54 | (R) CH(CH₃)—C(CH₃)₃ | CH₃ | Cl |
| A-55 | (R) CH(CH₃)—C(CH₃)₃ | CH₂CH₃ | Cl |
| A-56 | (±) CH(CH₃)—CF₃ | H | Cl |
| A-57 | (±) CH(CH₃)—CF₃ | CH₃ | Cl |
| A-58 | (±) CH(CH₃)—CF₃ | CH₂CH₃ | Cl |
| A-59 | (S) CH(CH₃)—CF₃ | H | Cl |
| A-60 | (S) CH(CH₃)—CF₃ | CH₃ | Cl |
| A-61 | (S) CH(CH₃)—CF₃ | CH₂CH₃ | Cl |
| A-62 | (R) CH(CH₃)—CF₃ | H | Cl |
| A-63 | (R) CH(CH₃)—CF₃ | CH₃ | Cl |
| A-64 | (R) CH(CH₃)—CF₃ | CH₂CH₃ | Cl |
| A-65 | (±) CH(CH₃)—CCl₃ | H | Cl |
| A-66 | (±) CH(CH₃)—CCl₃ | CH₃ | Cl |
| A-67 | (±) CH(CH₃)—CCl₃ | CH₂CH₃ | Cl |
| A-68 | (S) CH(CH₃)—CCl₃ | H | Cl |
| A-69 | (S) CH(CH₃)—CCl₃ | CH₃ | Cl |
| A-70 | (S) CH(CH₃)—CCl₃ | CH₂CH₃ | Cl |
| A-71 | (R) CH(CH₃)—CCl₃ | H | Cl |
| A-72 | (R) CH(CH₃)—CCl₃ | CH₃ | Cl |
| A-73 | (R) CH(CH₃)—CCl₃ | CH₂CH₃ | Cl |
| A-74 | CH₂CF₂CF₃ | H | Cl |
| A-75 | CH₂CF₂CF₃ | CH₃ | Cl |
| A-76 | CH₂CF₂CF₃ | CH₂CH₃ | Cl |
| A-77 | CH₂(CF₂)₂CF₃ | H | Cl |
| A-78 | CH₂(CF₂)₂CF₃ | CH₃ | Cl |
| A-79 | CH₂(CF₂)₂CF₃ | CH₂CH₃ | Cl |
| A-80 | CH₂C(CH₃)═CH₂ | H | Cl |
| A-81 | CH₂C(CH₃)═CH₂ | CH₃ | Cl |
| A-82 | CH₂C(CH₃)═CH₂ | CH₂CH₃ | Cl |
| A-83 | CH₂CH═CH₂ | H | Cl |
| A-84 | CH₂CH═CH₂ | CH₃ | Cl |
| A-85 | CH₂CH═CH₂ | CH₂CH₃ | Cl |
| A-86 | CH(CH₃)CH═CH₂ | H | Cl |
| A-87 | CH(CH₃)CH═CH₂ | CH₃ | Cl |
| A-88 | CH(CH₃)CH═CH₂ | CH₂CH₃ | Cl |
| A-89 | CH(CH₃)C(CH₃)═CH₂ | H | Cl |
| A-90 | CH(CH₃)C(CH₃)═CH₂ | CH₃ | Cl |
| A-91 | CH(CH₃)C(CH₃)═CH₂ | CH₂CH₃ | Cl |
| A-92 | CH₂—C≡CH | H | Cl |
| A-93 | CH₂—C≡CH | CH₃ | Cl |
| A-94 | CH₂—C≡CH | CH₂CH₃ | Cl |
| A-95 | cyclopentyl | H | Cl |
| A-96 | cyclopentyl | CH₃ | Cl |
| A-97 | cyclopentyl | CH₂CH₃ | Cl |
| A-98 | cyclohexyl | H | Cl |
| A-99 | cyclohexyl | CH₃ | Cl |
| A-100 | cyclohexyl | CH₂CH₃ | Cl |
| A-101 | CH₂—C₆H₅ | H | Cl |
| A-102 | CH₂—C₆H₅ | CH₃ | Cl |
| A-103 | CH₂—C₆H₅ | CH₂CH₃ | Cl |
| A-104 | NH₂ | CH₂—c—C₆H₁₁ | Cl |
| A-105 | NH₂ | CH₂CH₃ | Cl |
| A-106 | NH₂ | CH₂CH₂CH₃ | Cl |
| A-107 | NH—CH₂—CH═CH₂ | H | Cl |
| A-108 | NH—CH₂—CH═CH₂ | CH₃ | Cl |
| A-109 | NH—CH₂—CH═CH₂ | CH₂CH₃ | Cl |
| A-110 | NH—C(CH₃)₃ | H | Cl |
| A-111 | N(CH₃)₂ | H | Cl |
| A-112 | NH(CH₃) | H | Cl |
| A-113 | —(CH₂)₂CH═CHCH₂— | | Cl |
| A-114 | —(CH₂)₂C(CH₃)═CHCH₂— | | Cl |
| A-115 | —CH(CH₃)CH₂—CH═CHCH₂— | | Cl |
| A-116 | —(CH₂)₂CH(CH₃)(CH₂)₂— | | Cl |
| A-117 | —(CH₂)₃CHFCH₂— | | Cl |
| A-118 | —(CH₂)₂CHF(CH₂)₂— | | Cl |
| A-119 | —CH₂CHF(CH₂)₃— | | Cl |
| A-120 | —(CH₂)₂CH(CF₃)(CH₂)₂— | | Cl |
| A-121 | —(CH₂)₂O(CH₂)₂— | | Cl |
| A-122 | —(CH₂)₂S(CH₂)₂— | | Cl |
| A-123 | —(CH₂)₅— | | Cl |
| A-124 | —(CH₂)₄— | | Cl |
| A-125 | —CH₂CH═CHCH₂— | | Cl |
| A-126 | —CH(CH₃)(CH₂)₃— | | Cl |
| A-127 | —CH₂CH(CH₃)(CH₂)₂— | | Cl |
| A-128 | —CH(CH₃)—(CH₂)₂—CH(CH₃)— | | Cl |
| A-129 | —CH(CH₃)—(CH₂)₄— | | Cl |
| A-130 | —CH₂—CH(CH₃)—(CH₂)₃— | | Cl |
| A-131 | —(CH₂)—CH(CH₃)—CH₂—CH(CH₃)—CH₂— | | Cl |
| A-132 | —CH(CH₂CH₃)—(CH₂)₄— | | Cl |
| A-133 | —(CH₂)₂—CHOH—(CH₂)₂— | | Cl |
| A-134 | —(CH₂)₆— | | Cl |
| A-135 | —CH(CH₃)—(CH₂)₅— | | Cl |
| A-136 | —(CH₂)₂—N(CH₃)—(CH₂)₂— | | Cl |
| A-137 | —N═CH—CH═CH— | | Cl |
| A-138 | —N═C(CH₃)—CH═C(CH₃)— | | Cl |
| A-139 | —N═C(CF₃)—CH═C(CF₃)— | | Cl |
| A-140 | H | H | CH₃ |
| A-141 | CH₃ | H | CH₃ |
| A-142 | CH₃ | CH₃ | CH₃ |
| A-143 | CH₂CH₃ | H | CH₃ |
| A-144 | CH₂CH₃ | CH₃ | CH₃ |
| A-145 | CH₂CH₃ | CH₂CH₃ | CH₃ |
| A-146 | CH₂CF₃ | H | CH₃ |
| A-147 | CH₂CF₃ | CH₃ | CH₃ |
| A-148 | CH₂CF₃ | CH₂CH₃ | CH₃ |
| A-149 | CH₂CCl₃ | H | CH₃ |
| A-150 | CH₂CCl₃ | CH₃ | CH₃ |
| A-151 | CH₂CCl₃ | CH₂CH₃ | CH₃ |
| A-152 | CH₂CH₂CH₃ | H | CH₃ |
| A-153 | CH₂CH₂CH₃ | CH₃ | CH₃ |
| A-154 | CH₂CH₂CH₃ | CH₂CH₃ | CH₃ |
| A-155 | CH₂CH₂CH₃ | CH₂CH₂CH₃ | CH₃ |
| A-156 | CH(CH₃)₂ | H | CH₃ |
| A-157 | CH(CH₃)₂ | CH₃ | CH₃ |
| A-158 | CH(CH₃)₂ | CH₂CH₃ | CH₃ |
| A-159 | CH₂CH(CH₃)₂ | H | CH₃ |
| A-160 | CH₂CH(CH₃)₂ | CH₃ | CH₃ |
| A-161 | CH₂CH(CH₃)₂ | CH₂CH₃ | CH₃ |
| A-162 | CH₂CH(CH₃)₂ | CH₂CH₂CH₃ | CH₃ |
| A-163 | CH₂CH₂CH₂CH₃ | H | CH₃ |
| A-164 | CH₂CH₂CH₂CH₃ | CH₃ | CH₃ |
| A-165 | CH₂CH₂CH₂CH₃ | CH₂CH₃ | CH₃ |
| A-166 | CH₂CH₂CH₂CH₃ | CH₂CH₂CH₃ | CH₃ |
| A-167 | CH₂CH₂CH₂CH₃ | CH₂CH₂CH₂CH₃ | CH₃ |
| A-168 | (±) CH(CH₃)—CH₂CH₃ | H | CH₃ |
| A-169 | (±) CH(CH₃)—CH₂CH₃ | CH₃ | CH₃ |
| A-170 | (±) CH(CH₃)—CH₂CH₃ | CH₂CH₃ | CH₃ |
| A-171 | (S) CH(CH₃)—CH₂CH₃ | H | CH₃ |
| A-172 | (S) CH(CH₃)—CH₂CH₃ | CH₃ | CH₃ |
| A-173 | (S) CH(CH₃)—CH₂CH₃ | CH₂CH₃ | CH₃ |
| A-174 | (R) CH(CH₃)—CH₂CH₃ | H | CH₃ |
| A-175 | (R) CH(CH₃)—CH₂CH₃ | CH₃ | CH₃ |
| A-176 | (R) CH(CH₃)—CH₂CH₃ | CH₂CH₃ | CH₃ |
| A-177 | (±) CH(CH₃)—CH(CH₃)₂ | H | CH₃ |
| A-178 | (±) CH(CH₃)—CH(CH₃)₂ | CH₃ | CH₃ |
| A-179 | (±) CH(CH₃)—CH(CH₃)₂ | CH₂CH₃ | CH₃ |
| A-180 | (S) CH(CH₃)—CH(CH₃)₂ | H | CH₃ |
| A-181 | (S) CH(CH₃)—CH(CH₃)₂ | CH₃ | CH₃ |
| A-182 | (S) CH(CH₃)—CH(CH₃)₂ | CH₂CH₃ | CH₃ |
| A-183 | (R) CH(CH₃)—CH(CH₃)₂ | H | CH₃ |
| A-184 | (R) CH(CH₃)—CH(CH₃)₂ | CH₃ | CH₃ |
| A-185 | (R) CH(CH₃)—CH(CH₃)₂ | CH₂CH₃ | CH₃ |
| A-186 | (±) CH(CH₃)—C(CH₃)₃ | H | CH₃ |
| A-187 | (±) CH(CH₃)—C(CH₃)₃ | CH₃ | CH₃ |
| A-188 | (±) CH(CH₃)—C(CH₃)₃ | CH₂CH₃ | CH₃ |
| A-189 | (S) CH(CH₃)—C(CH₃)₃ | H | CH₃ |
| A-190 | (S) CH(CH₃)—C(CH₃)₃ | CH₃ | CH₃ |
| A-191 | (S) CH(CH₃)—C(CH₃)₃ | CH₂CH₃ | CH₃ |
| A-192 | (R) CH(CH₃)—C(CH₃)₃ | H | CH₃ |
| A-193 | (R) CH(CH₃)—C(CH₃)₃ | CH₃ | CH₃ |
| A-194 | (R) CH(CH₃)—C(CH₃)₃ | CH₂CH₃ | CH₃ |
| A-195 | (±) CH(CH₃)—CF₃ | H | CH₃ |
| A-196 | (±) CH(CH₃)—CF₃ | CH₃ | CH₃ |
| A-197 | (±) CH(CH₃)—CF₃ | CH₂CH₃ | CH₃ |
| A-198 | (S) CH(CH₃)—CF₃ | H | CH₃ |
| A-199 | (S) CH(CH₃)—CF₃ | CH₃ | CH₃ |
| A-200 | (S) CH(CH₃)—CF₃ | CH₂CH₃ | CH₃ |
| A-201 | (R) CH(CH₃)—CF₃ | H | CH₃ |
| A-202 | (R) CH(CH₃)—CF₃ | CH₃ | CH₃ |
| A-203 | (R) CH(CH₃)—CF₃ | CH₂CH₃ | CH₃ |
| A-204 | (±) CH(CH₃)—CCl₃ | H | CH₃ |
| A-205 | (±) CH(CH₃)—CCl₃ | CH₃ | CH₃ |
| A-206 | (±) CH(CH₃)—CCl₃ | CH₂CH₃ | CH₃ |
| A-207 | (S) CH(CH₃)—CCl₃ | H | CH₃ |
| A-208 | (S) CH(CH₃)—CCl₃ | CH₃ | CH₃ |
| A-209 | (S) CH(CH₃)—CCl₃ | CH₂CH₃ | CH₃ |
| A-210 | (R) CH(CH₃)—CCl₃ | H | CH₃ |
| A-211 | (R) CH(CH₃)—CCl₃ | CH₃ | CH₃ |
| A-212 | (R) CH(CH₃)—CCl₃ | CH₂CH₃ | CH₃ |
| A-213 | CH₂CF₂CF₃ | H | CH₃ |
| A-214 | CH₂CF₂CF₃ | CH₃ | CH₃ |
| A-215 | CH₂CF₂CF₃ | CH₂CH₃ | CH₃ |
| A-216 | CH₂(CF₂)₂CF₃ | H | CH₃ |
| A-217 | CH₂(CF₂)₂CF₃ | CH₃ | CH₃ |
| A-218 | CH₂(CF₂)₂CF₃ | CH₂CH₃ | CH₃ |
| A-219 | CH₂C(CH₃)═CH₂ | H | CH₃ |
| A-220 | CH₂C(CH₃)═CH₂ | CH₃ | CH₃ |
| A-221 | CH₂C(CH₃)═CH₂ | CH₂CH₃ | CH₃ |
| A-222 | CH₂CH═CH₂ | H | CH₃ |
| A-223 | CH₂CH═CH₂ | CH₃ | CH₃ |
| A-224 | CH₂CH═CH₂ | CH₂CH₃ | CH₃ |
| A-225 | CH(CH₃)CH═CH₂ | H | CH₃ |
| A-226 | CH(CH₃)CH═CH₂ | CH₃ | CH₃ |
| A-227 | CH(CH₃)CH═CH₂ | CH₂CH₃ | CH₃ |
| A-228 | CH(CH₃)C(CH₃)═CH₂ | H | CH₃ |
| A-229 | CH(CH₃)C(CH₃)═CH₂ | CH₃ | CH₃ |
| A-230 | CH(CH₃)C(CH₃)═CH₂ | CH₂CH₃ | CH₃ |
| A-231 | CH₂—C≡CH | H | CH₃ |
| A-232 | CH₂—C≡CH | CH₃ | CH₃ |
| A-233 | CH₂—C≡CH | CH₂CH₃ | CH₃ |
| A-234 | cyclopentyl | H | CH₃ |
| A-235 | cyclopentyl | CH₃ | CH₃ |
| A-236 | cyclopentyl | CH₂CH₃ | CH₃ |
| A-237 | cyclohexyl | H | CH₃ |
| A-238 | cyclohexyl | CH₃ | CH₃ |
| A-239 | cyclohexyl | CH₂CH₃ | CH₃ |
| A-240 | CH₂—C₆H₅ | H | CH₃ |
| A-241 | CH₂—C₆H₅ | CH₃ | CH₃ |
| A-242 | CH₂—C₆H₅ | CH₂CH₃ | CH₃ |
| A-243 | NH₂ | CH₂—c—C₆H₁₁ | CH₃ |
| A-244 | NH₂ | CH₂CH₃ | CH₃ |
| A-245 | NH₂ | CH₂CH₂CH₃ | CH₃ |
| A-246 | NH—CH₂—CH═CH₂ | H | CH₃ |
| A-247 | NH—CH₂—CH═CH₂ | CH₃ | CH₃ |
| A-248 | NH—CH₂—CH═CH₂ | CH₂CH₃ | CH₃ |
| A-249 | NH—C(CH₃)₃ | H | CH₃ |
| A-250 | N(CH₃)₂ | H | CH₃ |
| A-251 | NH(CH₃) | H | CH₃ |

| | | |
|---|---|---|
| A-252 | —(CH₂)₂CH═CHCH₂— | CH₃ |
| A-253 | —(CH₂)₂C(CH₃)═CHCH₂— | CH₃ |
| A-254 | —CH(CH₃)CH₂—CH═CHCH₂— | CH₃ |
| A-255 | —(CH₂)₂CH(CH₃)(CH₂)₂— | CH₃ |
| A-256 | —(CH₂)₃CHFCH₂— | CH₃ |
| A-257 | —(CH₂)₂CHF(CH₂)₂— | CH₃ |
| A-258 | —CH₂CHF(CH₂)₃— | CH₃ |
| A-259 | —(CH₂)₂CH(CF₃)(CH₂)₂— | CH₃ |
| A-260 | —(CH₂)₂O(CH₂)₂— | CH₃ |
| A-261 | —(CH₂)₂S(CH₂)₂— | CH₃ |
| A-262 | —(CH₂)₅— | CH₃ |
| A-263 | —(CH₂)₄— | CH₃ |
| A-264 | —CH₂CH═CHCH₂— | CH₃ |
| A-265 | —CH(CH₃)(CH₂)₃— | CH₃ |
| A-266 | —CH₂CH(CH₃)(CH₂)₂— | CH₃ |
| A-267 | —CH(CH₃)—(CH₂)₂—CH(CH₃)— | CH₃ |
| A-268 | —CH(CH₃)—(CH₂)₄— | CH₃ |
| A-269 | —CH₂—CH(CH₃)—(CH₂)₃— | CH₃ |
| A-270 | —(CH₂)—CH(CH₃)—CH₂—CH(CH₃)—CH₂— | CH₃ |
| A-271 | —CH(CH₂CH₃)—(CH₂)₄— | CH₃ |
| A-272 | —(CH₂)₂—CHOH—(CH₂)₂— | CH₃ |
| A-273 | —(CH₂)₆— | CH₃ |
| A-274 | —CH(CH₃)—(CH₂)₅— | CH₃ |
| A-275 | —(CH₂)₂—N(CH₃)—(CH₂)₂— | CH₃ |
| A-276 | —N=CH—CH=CH— | CH₃ |
| A-277 | —N=C(CH₃)—CH=C(CH₃)— | CH₃ |
| A-278 | —N=C(CF₃)—CH=C(CF₃)— | CH₃ |

The novel compounds of the formula I can be prepared analogously to known processes of the prior art.

For example, the compounds of the formula I can be prepared by reacting appropriately substituted 5-halo-4-aminopyrimidines II with appropriately substituted organometallic compounds III (see Scheme 1).

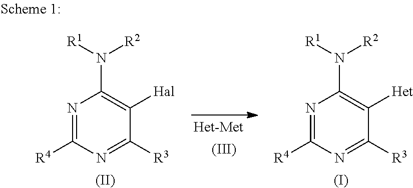

In Scheme 1, Het, R¹, R², R³ and R⁴ are as defined above, where R³ is typically not OH, Br or I. R³ is in particular hydrogen, alkyl, alkoxy, fluorine or chlorine; Hal is halogen, preferably bromine or iodine. Met is a radical attached via a metal atom, such as Sn, Zn or Mg, or a semimetal atom B, for example B(OH)₂ or B(OR)(OR′) where R, R′=C₁-C₄-alkyl, MgX where X=halogen, Zn—R″ where R″=alkyl or SnR₃ where R═C₁-C₄-alkyl.

The reaction is preferably carried out in the presence of catalytically active amounts of a transition metal of transition group VIII of the Periodic Table (group 10 according to IUPAC), for example nickel, palladium or platinum, in particular in the presence of a palladium catalyst. Suitable catalysts are, for example, palladium/phosphine complexes, such as tetrakis(triphenylphosphine)palladium(0), PdCl₂ (o-tolyl₃P)₂, bis(triphenylphosphine)palladium(II) chloride, the [1,1′-bis(diphenylphosphino)ferrocene]palladium(II) chloride/dichloromethane complex, bis[1,2-bis(diphenylphosphine)ethane]palladium(0) and [1,4-bis(diphenylphosphine)butane]palladium(II) chloride, palladium-on-carbon in the presence of phosphine compounds, and also palladium(II) compounds, such as palladium(II) chloride or bis(acetonitrile)palladium(II) chloride, in the presence of phosphine compounds, such as triphenylphosphine, 1,1′-bis(diphenylphosphino)ferrocene, 1,2-bis(diphenylphosphine)ethane, 1,3-bis(diphenylphosphine)propane and 1,4-bis(diphenylphosphine)butane. The amount of catalyst is usually from 0.1 to 20 mol %, based on the compound II.

Suitable organometallic compounds III are in particular appropriately substituted hetarylboronic acid and hetarylboronic esters (compounds III where Met=B(OH)₂ or B(OR)(OR′) where R, R′=C₁-C₄-alkyl). Also suitable are compounds Het-Met which represent a corresponding boronic anhydride of the formula

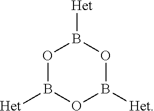

The reaction is carried out under the conditions of a Suzuki coupling as known, for example, from Suzuki et al., Chem. Rev., 1995, 95, 2457-2483 and the literature cited therein. The hetarylboronic acids and their esters can be prepared from the corresponding hetaryllithium compounds or hetarylmagnesium compounds by reaction with boronic esters B(OR)₃ where R═C₁-C₄-alkyl. Hetaryllithium compounds for their part can be prepared by direct metallation of CH-acidic heteroaromatic compounds with lithium bases such as lithium diisopropylamide or butyllithium, or by lithiation of halohetaryl compounds with alkyllithium, such as n-butyllithium.

Other suitable organometallic compounds III are hetarylstannanes (compounds III where Met=SnR₃ where R=C₁-C₄-alkyl). In this case, the reaction is carried out under the conditions of a Stille coupling as known, for example, from D. Milstein, J. K. Stille, J. Am. Chem. Soc. 1978, 100, pp. 3636-3638 or V. Farina, V. Krishnamurthy, W. J. Scott, Org. React. 1997, 50, 1-652. Hetarylstannanes III can be prepared analogously to known processes by reacting hetaryllithium compounds with R₃SnCl.

Suitable organometallic compounds III are furthermore Grignard reagents (compounds III where Met=Mg-Hal′ where Hal′=Cl, Br, in particular Br). In this case, the reaction is carried out under the conditions of a Kumada coupling as known, for example, from Kumada, Tetrahedron, 1982, 38, 3347 or A. C. Frisch, N. Shaikh, A. Zapf, M. Beller, Angew. Chem., 2002, 114, 4218-4221.

Suitable organometallic compounds III are furthermore organozinc compounds (compounds III where Met=Zn—Hal′ where Hal′=Cl, Br, in particular Br). In this case, the reaction is carried out under the conditions of a Negishi coupling as known, for example, from A. Lützen, M. Hapke, Eur. J. Org. Chem., 2002, 2292-2297. Hetarylzinc compounds can be prepared in a manner known per se from the hetaryllithium compounds or the hetarylmagnesium compounds by reaction with zinc salts, such as zinc chloride.

In particular in the case of a Suzuki coupling, the reaction of II with the organometallic compound III is carried out under basic conditions. Suitable bases are alkali metal carbonates and alkali metal bicarbonates, such as sodium carbonate, potassium carbonate, cesium carbonate, sodium bicarbonate, alkaline earth metal carbonates and alkaline earth metal bicarbonates, such as magnesium carbonate or magnesium bicarbonate, or tertiary amines, such as triethylamine, trimethylamine, triisopropylamine or Nethyl-N-diisopropylamine.

The coupling of the compound II with the compound III is usually carried out in a solvent. Suitable solvents are organic solvents, such as ethers, for example, 1,2-dimethoxyethane, cyclic ethers, such as tetrahydrofuran or 1,4-dioxane, polyalkylene glycols, such as diethylene glycol, carbonitriles, such as acetonitrile, propionitrile, carboxamides, such as dimethylformamide or dimethylacetamide. In the Suzuki coupling the solvents, mentioned above may also be used as a mixture with water, the ratio of organic solvent to water may, for example, be in the range from 5:1 to 1:5.

Advantageously, the compounds II in which R⁴ is cyano or a group attached via a heteroatom, such as hydroxyl, mercapto, azido, alkoxy, alkenyloxy, alkynyloxy, haloalkoxy, alkylthio, alkenylthio, alkynylthio, haloalkylthio, ON(═CR⁴⁹R⁵⁰), O—C(═Z)R⁴⁵NR⁴²R^{43a}, NR⁵¹(C(═Z)R⁴⁵), NR⁵¹(C(═Z)OR⁴¹), NR⁵¹(C(═Z)—NR⁴²R⁴³), NR⁵²(N═CR⁴⁹R⁵⁰), NR⁵²NR⁴²R⁴³ or NR⁵²OR⁴¹, can be advantageously obtained from the appropriately substituted sulfones IV (see Scheme 2).

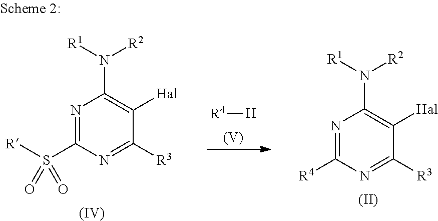

In Scheme 2, R¹, R², R³ and R⁴ are as defined above. R³ is in particular alkyl or halogen. R′ is C₁-C₆-alkyl, and Hal is halogen, preferably bromine or iodine.

In general, the sulfones of the formula IV are reacted with compounds V under basic conditions. For practical reasons, the alkali metal, alkaline earth metal or ammonium salt of the compound V may be employed directly. Alternatively, base may be added.

This reaction is typically carried out under the conditions of a nucleophilic substitution; usually at from 0 to 200° C., preferably at from 10 to 150° C. If appropriate, it may be advantageous to carry out the reaction in the presence of a phase transfer catalyst, for example 18-crown-6. The reaction is usually carried out in the presence of a dipolar aprotic solvent, such as N,N-dialkylated carboxamides, for example N,N-dimethylformamide, cyclic ethers, for example tetrahydrofuran, or carbonitriles, such as acetonitrile [cf. DE-A 39 01 084; Chimia, Vol. 50, pp. 525-530 (1996); Khim. Geterotsikl. Soedin, Vol. 12, pp. 1696-1697 (1998)].

In general, the compounds IV and V are employed in approximately stoichiometric amounts. However, it may be advantageous to use an excess of the nucleophile of the formula R⁴—H, for example an up to 10-fold, in particular up to 3-fold, excess, based on the compound II.

In general, the reaction is carried out in the presence of a base which may be employed in equimolar amounts or else in excess. Suitable bases are alkali metal carbonates and bicarbonates, for example sodium carbonate and sodium bicarbonate, nitrogen bases, such as triethylamine, tributylamine and pyridine, alkali metal alkoxides, such as sodium methoxide or potassium tert-butoxide, alkali metal amides, such as sodium amide, or alkali metal hydrides, such as lithium hydride or sodium hydride.

Suitable solvents are halogenated hydrocarbons, ethers, such as diethyl ether, diisopropyl ether, tert-butyl ether, 1,2-dimethoxyethane, dioxane, anisole and tetrahydrofuran, and also dimethyl sulfoxide, N,N-dialkylated carboxamides, such as dimethylformamide or dimethylacetamide. Particular preference is given to ethanol, dichloromethane, acetonitrile and tetrahydrofuran. It is also possible to use mixtures of the solvents mentioned.

Compounds IV in which R⁴ is cyano are useful intermediates for preparing further compounds I.

Compounds II in which R⁴ is a derivatized carboxylic acid radical, such as C(═O)OR⁴¹, C(═O)NR⁴²R⁴³, C(═NOR⁵⁴)NR⁴²R⁴³, C(═O)NR⁴⁴—NR⁴²R⁴³, C(═N—NR⁵⁵R⁵⁶)NR⁴²R⁴³, C(═NOR⁵⁴)NR⁴⁴—NR⁴²R⁴³, C(═O)R⁴⁵, CR⁴⁶R⁴⁷—OR⁴⁸, CR⁴⁶R⁴⁷—NR⁴²R⁴³ can be obtained in an advantageous manner from compounds II in which R⁴ is cyano, by standard processes for derivatizing CN groups.

Compounds II in which R⁴ is C(═O)NR⁴²R⁴³ can be obtained from compounds II in which R⁴ is cyano, by hydrolysis to give the carboxylic acids (where R⁴═COOH) under acidic or basic conditions and amidation with amines VI, HNR⁴²R⁴³, see Scheme 2a.

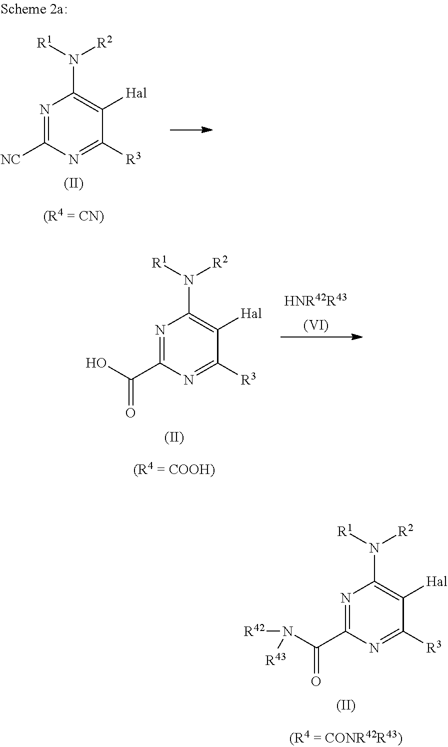

In Scheme 2a, R¹, R², R³, R⁴², R⁴³ are as defined above. R³ is in particular alkyl or halogen, Hal is halogen, preferably bromine or iodine. The hydrolysis of the nitrile II (R⁴=CN) is usually carried out in inert polar solvents, such as water or alcohols, preferably using inorganic bases, such as alkali metal or alkaline earth metal hydroxides, in particular NaOH. In a preferred embodiment, the nitrile II is hydrolyzed by reaction with hydrogen peroxide under alkaline conditions.

The reaction of the acid II (R⁴═COOH) with the amine VI is advantageously carried out under the conditions known from Chem. and Pharm. Bull. 1982, Vol. 30, N12, p. 4314. If appropriate, it may be advantageous to activate the acid II prior to the reaction with the amine VI, for example to convert it into its acid chloride. In the case of carboxylic acids II prone to decarboxylation, it may be advantageous not to isolate the free acid but to convert its alkali metal salt directly with customary halogenating agents, for example with oxalyl chloride, into the acid chloride, and to react the latter with the amine, if appropriate in the presence of an auxiliary base.

Alternatively, the amides II can be prepared by standard methods from corresponding imino esters (R⁴═C(═NH)OR⁴¹), which for their part can be prepared by acidic hydrolysis of the nitriles II in alcoholic solvents.

Amides of the formula II (where R⁴═CONR⁴²R⁴³) afford, by oximation with hydroxylamine or substituted hydroxylamines H₂N—OR⁵⁴ under basic conditions, the compounds of the formula II in which R⁴ is C(═NOR⁵⁴)NR⁴²R⁴³ [cf. U.S. Pat. No. 4,876,252]. The substituted hydroxylamines can be employed as free base or, preferably, in the form of their acid addition salts. For practical reasons, the halides, such as chlorides, or the sulfates are particularly suitable.

Alternatively, the amidoximes of the formula II in which R⁴ is C(═NOR⁵⁴)NR⁴²R⁴³ can also be prepared from the corresponding nitriles II by reaction with hydroxylamine or substituted hydroxylamines H₂N—OR⁵⁴ under basic conditions, see Scheme 2b. This reaction is advantageously carried out under the conditions known from DE-A 198 37 794. The resulting compounds II in which R⁴ is C(═NOR⁵⁴)NH₂ can be mono- or dialkylated, giving the compounds C(═NOR⁵⁴)NR⁴²R⁴³ in which R⁴² and/or R⁴³ are different from hydrogen. Suitable alkylating agents are, for example, C₁-C₆-alkyl halides, di-C₁-C₆-alkyl sulfates or C₁-C₆-alkyl phenolsulfonates, where the phenyl radical optionally carries one or two radicals selected from the group consisting of nitro and C₁-C₆-alkyl. The alkylation is usually carried out in the presence of a base. Suitable bases are, in principle, all compounds capable of deprotonating the amide nitrogen. Suitable bases are, for example, alkali metal or alkaline earth metal hydroxides, such as sodium hydroxide, potassium hydroxide or lithium hydroxide.

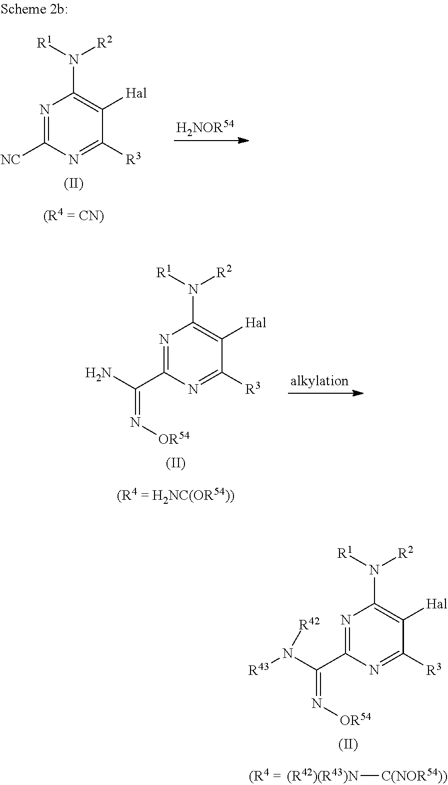

In Scheme 2b, R¹, R², R³, R⁴², R⁴³, R⁵⁴ are as defined above, R³ is in particular alkyl or halogen and Hal is halogen, preferably bromine or iodine.

Compounds of the formula II in which R⁴ is C(═N—NR⁵⁵R⁵⁶)NR⁴²R⁴³ can be prepared in an advantageous manner from the corresponding cyano compounds II by reaction with H₂N—NR⁵⁵R⁵⁶ to give the corresponding compounds II in which R⁴═C(═N—NR⁵⁵R⁵⁶)NH₂. The compounds obtained in this manner can be mono- or dialkylated, which gives compounds II in which R⁴ is C(═N—NR⁵⁵R⁵⁶)NR⁴²R⁴³ and in which R⁴² and/or R⁴³ are different from hydrogen. With respect to suitable alkylation processes, reference is made to what has been stated above.

Compounds of the formula II in which R⁴ is C(═O)R⁴⁵ are obtainable from the corresponding cyano compounds II by reaction with Grignard reagents R⁴⁵—Mg-Hal, in in which Hal is a halogen atom, in particular chlorine or bromine. This reaction is advantageously carried out under the conditions known from J. Heterocycl. Chem. 1994, Vol. 31(4), p. 1041.

Compounds of the formula II in which R⁴ is CR⁴⁶R⁴⁷—OR⁴⁸ can be obtained from the corresponding ketones in which R⁴ is C(═O)R⁴⁵ by reaction with Grignard reagents R⁴⁶R⁴⁷—Mg-Hal* in which Hal* is a halogen atom, in particular chlorine or bromine, and, if appropriate, subsequent alkylation.

Compounds of the formula II in which R⁴ is CH₂—OR⁴⁸ can be obtained from the corresponding ketones in which R⁴ is C(═O)R⁴⁵ by reduction with a metal hydride, for example lithium aluminum hydride, and, if appropriate, subsequent alkylation.

Compounds of the formula II in which R⁴ is C(═N—NR⁵⁵R⁵⁶)R⁴⁵ can be obtained via compounds II (where R⁴═C(═O)R⁴⁵) which are reacted with hydrazines H₂NNR⁵⁵R⁵⁶, preferably under the conditions known from J. Org. Chem. 1966, Vol. 31, p. 677.

Compounds of the formula II in which R⁴ is C(═NOR⁵⁴)R⁴⁵ can be obtained by oximation of compounds II (R⁴═C(═O)R⁴⁵). The oximation is carried out as described above.

Compounds of the formula II in which R⁴ is C(═O)OR⁴¹ can be obtained by esterification of the compounds II (R⁴═COOH) under acidic or basic conditions.

Compounds of the formula II in which R⁴ is C(═S)NR⁴²R⁴³ can be obtained by reacting compounds II in which R⁴ is CN, see Scheme 2c.

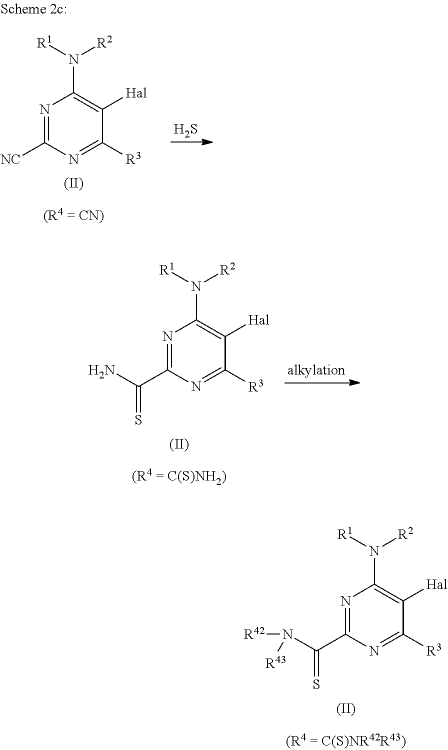

In Scheme 2c, R¹, R², R³, R⁴², R⁴³ are as defined above. R³ is in particular alkyl or halogen, Hal is halogen, preferably bromine or iodine. In general, the cyano compound II is reacted in the presence of a solvent or diluent with hydrogen sulfide gas. Suitable solvents or diluents are, for example, aromatic amines, such as pyridine, substituted pyridines, such as collidine and lutidine, or tertiary amines, such as trimethylamine, triethylamine, triisopropylamine and N-methylpiperidine. The aminothiocarbonyl compounds II (R⁴═C(═S)NH₂) obtained in this manner can then, if appropriate, be mono- or dialkylated at the amide nitrogen. With respect to suitable processes for the alkylation, reference is made to what was stated above.

Alternatively, compounds II in which R⁴ is C(═S)NR⁴²R⁴³ can be obtained by sulfurization from the corresponding carboxamide compounds II (compounds II with C(═O)NR⁴²R⁴³). Examples of suitable sulfurizing agents are organophosphorus sulfides, such as Lawesson's reagent, (2,2-bis(4-methoxyphenyl)-1,3,2,4-dithiodiphosphetane 2,4-disulfide, organotin sulfides, such as bis(tricyclohexyltin) sulfide, or phosphorus pentasulfide (see also J. March, Advanced Organic Chemistry, 4th edition, Wiley Interscience 1992, p. 893f and the literature cited therein).

Compounds IV can be prepared, for example, according to the synthesis shown in Scheme 3 by oxidation of the thioethers VII.

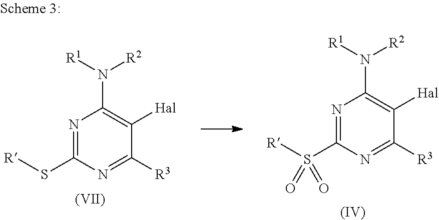

In Scheme 3, R¹, R² and R³ are as defined above. R³ is in particular alkyl or halogen. Hal is halogen, preferably bromine or iodine, and R′ is C₁-C₆-alkyl.

Suitable oxidizing agents are, for example, hydrogen peroxide, selenium dioxide [cf. WO 02/88127] or organic carboxylic acids, such as 3-chloroperbenzoic acid. The oxidation is preferably carried out at from 10 to 50° C. in the presence of protic or aprotic solvents [cf. B. Kor. Chem. Soc., Vol. 16, pp. 489-492 (1995); Z. Chem., Vol. 17, p. 63 (1977)].

Compounds VII in which Hal is halogen, in particular bromine or iodine, can be obtained, for example, according to the synthesis route outlined in Scheme IV.

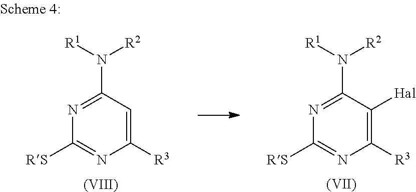

In Scheme 4, R³, R¹ and R² are as defined above. R³ is in particular alkyl or halogen, R¹ is C₁-C₆-alkyl. Hal is halogen, preferably bromine or iodine.

The 4-aminopyridines VIII can be converted by customary methods into the 4-amino-5-halopyrimidines VII. Suitable halogenating agents are, preferably, chlorinating agents, brominating agents and iodinating agents. A suitable chlorinating agent is, for example, N-chlorosuccinimide. Suitable brominating agents are bromine and N-bromosuccinimide. The bromination is usually carried out in the presence of a solvent. Suitable solvents for the bromination are, for example, carboxylic acids, such as acetic acid. Suitable iodinating agents are hydrogen iodide, iodine monochloride or N-iodosuccinimide. The iodination is usually carried out in a solvent. Suitable solvents are chlorinated hydrocarbons, such as dichloromethane, if hydrogen iodide is used, C₁-C₄-alkohols, such as methanol or carboxylic acids, such as acetic acid, if iodine monochloride is used, and halogenated carboxylic acids, such as trifluoroacetic acid, if N-iodosuccinimide is used. The halogenation is usually carried out between 10° C. and the boiling point of the solvent.

4-Aminopyrimidine compounds VIII can be prepared from 4-halopyrimidine compounds IX by reaction with a primary or secondary amine (compound X) (see Scheme 5).

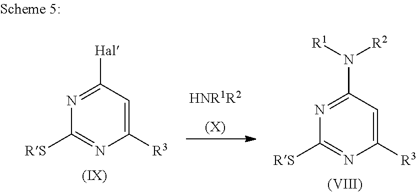

In Scheme 5, R³, R¹ and R² are as defined above. R³ is in particular halogen or alkyl, R′ is C₁-C₆-alkyl, and Hal′ is halogen, in particular chlorine. The reaction is advantageously carried out at from 0 to 70° C., preferably from 10 to 35° C. The reaction is usually carried out in an inert solvent, such as an ether, for example dioxane, tetrahydrofuran or diethyl ether, a halogenated hydrocarbon, such as dichloromethane, an aromatic hydrocarbon, for example toluene, or a carboxylic ester, such as ethyl acetate [cf. WO 98/46608]. If appropriate, it may be advantageous to carry out the reaction in the presence of a base, such as a tertiary amine, for example triethylamine or an inorganic base, such as an alkali metal or alkaline earth metal carbonate, an alkali metal or alkaline earth metal bicarbonate; it is also possible for excess amine X to serve as base.

4-Halopyrimidines IX in which R³ is alkyl are advantageously obtained by reacting 4,6-dihalopyrimidines XI with a Grignard reagent R³—MgCl under the conditions of a Kumada coupling, as described in Scheme 6.

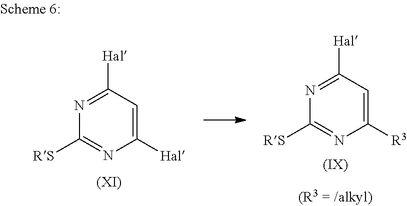

In Scheme 6, Hal′ independently of one another are halogen, preferably chlorine.

4,6-Dihalopyrimidines XI are, for example, obtained in an advantageous manner by reacting 4,6-dihydroxypyrimidines XII with halogenating agents, in particular chlorinating agents or brominating agents, as described in Scheme 7.

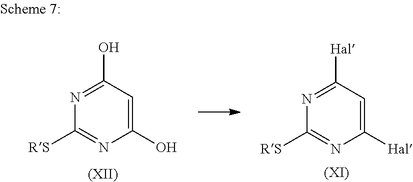

In Scheme 7, Hal′ are halogen, preferably chlorine. Suitable chlorinating agents for the conversion of the dihydroxy compound XII into the compounds XI are in particular POCl₃, PCl₃/Cl₂ or PCl₅, or mixtures of these reagents. The reaction can be carried out in excess chlorinating agent (POCl₃) or in an inert solvent, such as, for example, a carbonitrile, for example, acetonitrile or propionitrile, an aromatic hydrocarbon, for example toluene, a chlorinated hydrocarbon, for example 1,2-dichloroethane, or a chlorinated aromatic hydrocarbon, such as chlorobenzene.

The reaction is generally carried out between 10 and 180° C. Advantageously, the process is carried out with addition of N,N-dimethylformamide in catalytic or subcatalytic amounts or of nitrogen bases, such as, for example, N,N-dimethylaniline.

4,6-Dihydroxypyrimidines XII can be obtained, for example, by initially converting malonic esters XIV with thiourea into the 2-mercaptopyrimidine compound XIII. Subsequent alkylation with an alkylating agent gives the compound XII. Suitable alkylating agents are, for example, C₁-C₆-alkyl halides, preferably alkyl bromides and alkyl chlorides, di-C₁-C₆-alkyl sulfates or C₁-C₆-alkyl phenolsulfonates. Usually, the reaction is carried out in the presence of a solvent which is inert under the reaction conditions.

Alternatively, the dihydroxypyrimidine compound XII can also be reacted directly with a S-alkyl isothiourea, giving the thioether XII directly, see Scheme 8.

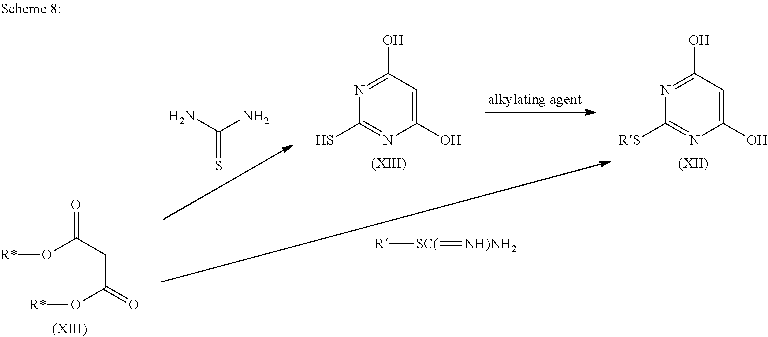

In Scheme 8, R* is alkyl, preferably C₁-C₆-alkyl, and R′ is C₁-C₆-alkyl.

Alternatively, compounds IX in which R³ is alkyl can be obtained by the route shown in Scheme 9.

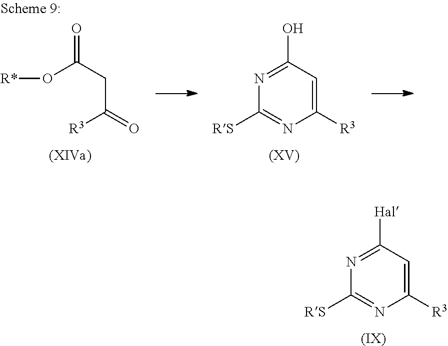

In Scheme 9, R* is alkyl, preferably C₁-C₆-alkyl, R′ is C₁-C₆-alkyl and Hal′ is halogen, preferably chlorine Initially, an appropriately substituted f-keto ester of the formula XIVa is, under the conditions described in Scheme 8, converted into a 2-thioetherpyrimidine compound XV. The compound XV is then reacted with a halogenating agent under the conditions described in Scheme 7 to give a 4-halopyrimidine of the formula IX.

Compounds of the formula I in which R³ is cyano, C₁-C₈-alkoxy, C₁-C₈-alkylthio or C₁-C₈-haloalkoxy can be obtained in an advantageous manner by reacting compounds I in which R³ is halogen, preferably chlorine, with compounds M¹-R³*(hereinbelow also compounds of the formula XVI). The compounds of the formula XVI are, depending on the groups R³* to be introduced, inorganic cyanides, alkoxides, thiolates or haloalkoxides. The reaction is advantageously carried out in an inert solvent. The cation M¹ in formula XVI is of little importance; for practical reasons, ammonium salts, tetraalkylammonium salts, such as tetramethylammonium or tetraethylammonium salts, or alkali metal or alkaline earth metals are usually preferred (Scheme 10).

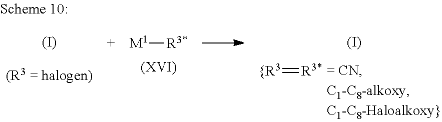

The reaction temperature is usually from 0 to 120° C., preferably from 10 to 40° C. [cf. J. Heterocycl. Chem., Vol. 12, pp. 861-863 (1975)].

Suitable solvents include ethers, such as dioxane, diethyl ether, methyl tert-butyl ether and, preferably, tetrahydrofuran, halogenated hydrocarbons, such as dichloromethane or dichloroethane, aromatic hydrocarbons, such as toluene, and mixtures thereof.

Compounds of the formula I in which R³ is C₁-C₈-alkyl, C₁-C₈-haloalkyl, C₂-C₈-alkenyl, C₂-C₈-haloalkenyl, C₂-C₈-alkynyl or C₂-C₈-haloalkynyl can be prepared in an advantageous manner by reacting compounds I in which R³ is halogen, in particular chlorine, with organometallic compounds X^{a}-Mt in which X^{a} is C₁-C₈-alkyl, C₁-C₈-haloalkyl, C₂-C₈-alkenyl, C₂-C₈-haloalkenyl, C₂-C₈-alkynyl or C₂-C₈-haloalkynyl and Mt is lithium, magnesium or zinc. The reaction is preferably carried out in the presence of catalytic or, in particular, at least equimolar amounts of transition metal salts and/or compounds, in particular in the presence of Cu salts, such as Cu(I) halides and especially Cu(I) iodide. In general, the reaction is carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents. The temperatures required for this purpose are in the range of from −100 to +100° C. and especially in the range of from −80° C. to +40° C. Corresponding processes are known, for example from WO 03/004465

By way of example, the synthesis of the compound I in which R⁴ is a radical C(═NOR⁵⁴)NH₂ and R³ is alkyl is shown in Scheme 11.

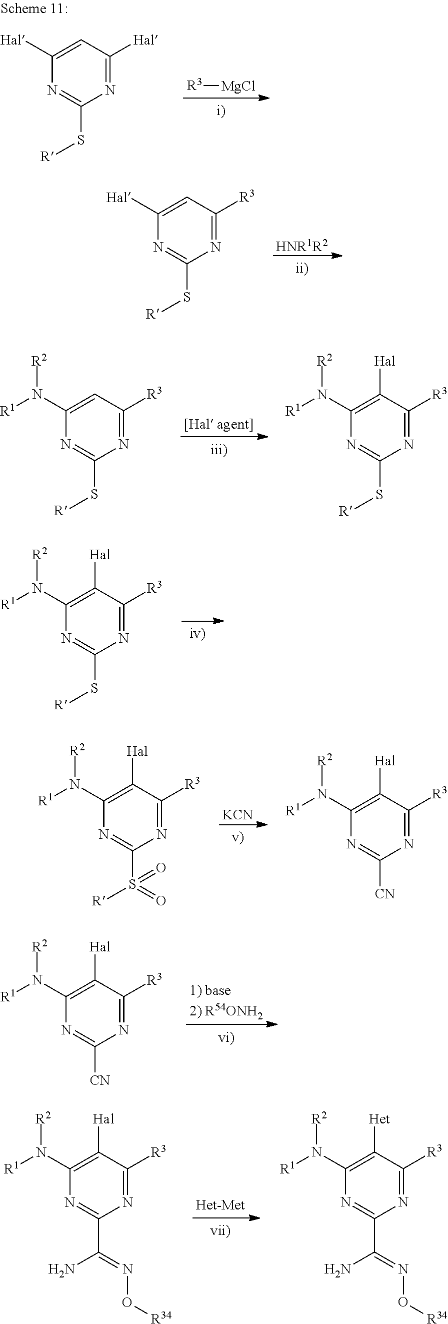

In Scheme 11, Hal′ is halogen, preferably chlorine; Hal is halogen, preferably bromine or iodine; Hal′ agent is a halogenating agent; Met is a radical attached via a metal atom or a semimetal atom.

Step i) is carried out as described in Scheme 6.
Step ii) is carried out as described in Scheme 5.
Step iii) is carried out as described in Scheme 4.
Step iv) is carried out as described in Scheme 3.
Step v) is carried out as described in Scheme 2.
Step vi) is carried out as described in Scheme 2b.
Step vii) is carried out as described in Scheme 1.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after use, for example, in the case of treatment of plants, in the treated plants, or in the harmful fungus to be controlled.

The compounds of the formula I are suitable for use as fungicides. They are distinguished by excellent activity against a broad spectrum of phytopathogenic fungi from the classes of the Ascomycetes, Deuteromycetes, Oomycetes and Basidiomycetes, in particular from the class of the Oomycetes. Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetables, such as cucumbers, beans, tomatoes, potatoes and cucurbits, and also on the seeds of these plants. They can also be used in crops which are tolerant towards insecticidal or fungal attack due to breeding, including genetical modifications. Moreover, they are useful for controlling Botryospheria species, Cylindrocarpon species, Eutypa lata, Neonectria liriodendri and Stereum hirsutum which inter alia attack the wood and roots of grapevines.

They are especially suitable for controlling the following plant diseases:
Alternaria species on vegetables, rapeseed, sugar beet, fruit, rice, soybeans and also on potatoes (for example A. solani or A. alternata) and tomatoes (for example A. solani or A. alternata) and Alternaria ssp. (black mould) on wheat,
Aphanomyces species on sugar beet and vegetables,
Ascochyta species on cereals and vegetables, for example Ascochyta tritici (speckled leaf blotch) on wheat.
Bipolaris and Drechslera species on corn, cereals, rice and lawns (for example D. maydis on corn, D. teres on barley, D. tritci-repentis on wheat),
Blumeria graminis (powdery mildew) on cereals (for example wheat or barley),
Botrytis cinerea (gray mold) on strawberries, vegetables, flowers, wheat and grapevines,
Bremia lactucae on lettuce,
Cercospora species on corn, soybeans, rice and sugar beet and for example Cercospora sojina (leaf blotch) or Cercospora kikuchii (leaf blotch) on soybeans,
Cladosporium herbarum (black mould) on wheat
Cochliobolus species on corn, cereals, rice (for example Cochliobolus sativus on cereals, Cochliobolus miyabeanus on rice),
Colletotricum species on soybeans, cotton and other plants (for example C. acutatum on various plants) Colletotricum truncatum (antracnosis) on soybeans,
Corynespora cassiicola (leaf blotch) on soybeans,
Dematophora necatrix (root/stem rot) on soybeans,
Diaporthe phaseolorum (stem rot) on soybeans,
Drechslera species, Pyrenophora species on corn, cereals, rice and lawns, on barley (for example D. teres) or on wheat (for example D. tritici-repentis),
Esca on grapevines, caused by Phaeoacremonium chlamydosporium, Ph. Aleophilum, and Formitipora punctata (syn. Phellinus punctatus),
Elsinoe ampelina on grapevines,
Epicoccum spp. (black mould) on wheat,
Exserohilum species on corn,
Erysiphe cichoracearum and Sphaerotheca fuliginea on cucurbits,
Fusarium and Verticillium species (for example V. dahliae) on various plants: for example F. graminearum or F. culmorum (root rot) on cereals (e.g. wheat or barley) or for example F. oxysporum on tomatoes or F. solani (stem rot) on soybeans,
Gaeumanomyces graminis on cereals (for example wheat or barley),
Gibberella species on cereals and rice (for example Gibberella fujikuroi on rice),
Grainstaining complex on rice,
Guignardia budwelli on grapevines,
Helminthosporium species (for example H. graminicola) on corn and rice,
Isariopsis clavispora on grapevines,
Macrophomina phasolina (root/stem rot) on soybeans.
Michrodochium nivale on cereals (for example wheat or barley),
Microsphera diffusa (powdery mildew) on soybeans,
Mycosphaerella species on cereals, bananas and peanuts (M. graminicola on wheat, M. fijiesis on bananas),
Peronospora species on cabbage (for example P. brassicae), bulbous plants (for example P. destructor) and for example P. manshurica (downy mildew) on soybeans,
Phakopsara pachyrhizi and Phakopsara meibomiae on soybeans,
Phialophora gregata (stem rot) on soybeans,
Phomopsis species on soybeans, sunflowers and grapevines (P. viticola on grapevines, P. helianthi on sunflowers),
Phytophthora species on various plants, for example P. capsici on bell peppers, Phytophthora megasperma (leaf blight/stem rot) on soybeans, Phytophthora infestans on tomatoes and potatoes,
Plasmopara viticola on grapevines,
Podosphaera leucotricha on apples,
Pseudocercosporella herpotrichoides on cereals,
Pseudoperonospora species on hops and cucurbits (for example P. cubenis on cucumbers or P. humili on hops),
Pseudopezicula tracheiphilai on grapevines,
Puccinia species on various plants, for example P. triticina, P. striformins, P. hordei or P. graminison cereals (for example wheat or barley), or on asparagus (for example P. asparagi), -Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants (for example P. ultiumum or P. aphanidermatum),
Ramularia collo-cygni (Ramularia/physiological leaf spots) on barley,
Rhizoctonia-species (for example R. solani) on cotton, rice, potatoes, lawns, corn, rapeseed, potatoes, sugar beet, vegetables and other plants, for example Rhizoctonia solani) (root/stem rot) on soybeans or Rhizoctonia cerealis (sharp eyespot) on wheat or barley,
Rhynchosporium secalis on barley (leaf blotch), rye and triticale,
Sclerotinia species on rapeseed, sunflowers and other plants, for example S. sclerotiorum (stem rot) or Sclerotinia rolfsii (stem rot) on soybeans,
Septoria glycines (leaf blotch) on soybeans,
Septoria tritici and Stagonospora nodorum on wheat,
Erysiphe (syn. Uncinula necator) on grapevines,
Setospaeria species on corn and lawns,
Sphacelotheca reilinia on corn,
Stagonospora nodorum (leaf blotch) on wheat,
Thievaliopsis species on soybeans and cotton,
Tilletia species on cereals,
Typhula incarnata (snow rot) on wheat and barley,
Ustilago species on cereals, corn and sugar beet and
Venturia species (scab) on apples and pears (for example V. inaequalis on apples).

The compounds of the formula I are furthermore suitable for controlling harmful fungi in the protection of materials (for example wood, paper, paint dispersions, fibers or fabrics) and in the protection of stored products. In the protection of wood, particular attention is paid to the following harmful fungi:

Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds of the formula I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in different crystal modifications which may differ in their biological activity. They also form part of the subject matter of the present invention.

The compounds of the formula I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, atta clay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for Dilution with Water

### A Water-Soluble Concentrates (SL, LS)

10 parts by weight of the active compound are dissolved in 90 parts by weight of water or in a water-soluble solvent. As an alternative, wetters or other auxiliaries are added. The active compound dissolves upon dilution with water. In this way, a formulation having a content of 10% by weight of active compound is obtained.

### B Dispersible Concentrates (DC)

20 parts by weight of the active compound are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of the active compound are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compound are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturax) and made into a homogeneous emulsion. Dilution with water gives an emulsion. The formulation has an active compound content of 25% by weight.

### E Suspensions (SC, OD, FS)

In an agitated ball mill, 20 parts by weight of the active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compound are ground finely with addition of 50 parts by weight of dispersants and wetters and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of the active compound, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compound are ground finely and mixed intimately with 95 parts by weight of finely divided kaolin. This gives a dustable product having an active compound content of 5% by weight.

### J Granules (GR, FG, GG, MG)

0.5 part by weight of the active compound is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

parts by weight of the active compound are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (wettable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even to apply the active compound without additives.

Oils of various types, wetting agents, adjuvants, herbicides, fungicides, other pesticides, or bactericides may be added to the active compounds, even, if appropriate, not until immediately prior to use (tank mix). These agents may be admixed with the compositions according to the invention in a weight ratio of from 1:100 to 100:1, preferably from 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, for example with herbicides, insecticides, growth regulators, fungicides or also with fertilizers. By mixing the compounds (I) or the compositions comprising them in the application form as fungicides with other fungicides, it is in many cases possible to broaden the fungicidal activity spectrum.

The following list of fungicides, with which the compounds according to the invention can be used in conjunction, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, pen-βthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

Moreover, the compounds of the formula I according to the invention and salts thereof, in particular the agriculturally acceptable salts thereof are suitable for controlling arthropod plant pests, in particular plant-damaging insects and arachnids. Furthermore, the compounds of the formula I according to the invention and salts thereof, in particular the agriculturally acceptable salts thereof are suitable for controlling nematodes, in particular plant-damaging nematodes.

Examples of plant-damaging arthropods are insects
of the order Lepidoptera, for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis plleriana, Spodoptera eridania, Spodoptera frugiperda, Spodoptera littoralis, Spodoptera litura, Thaumatopoea pityocampa, Tortrix viridana, Trichoplusia ni and Zeiraphera canadensis,
of the order Coleoptera (beetles), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi Chaetocnema tibalis, Conoderus vespertinus, Crioceris asparag Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastan/Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,
of the order Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis ceras, Rhagoletis pomonella, Tabanus bovinus, Tipula oleracea and Tipula paludosa,
of the order Thysanoptera (thrips), for example Dichromothrips spp., Frankliniella fusca, Frankliniella occidentalis, Frankliniella tritici, Scirtothrips citri, Thrips oryzae, Thrips palmi and Thrips tabaci,
of the order Hymenoptera, for example Athalia rosae, Atta cephalotes, Atta sexdens, Atta texana, Hoplocampa minuta, Hoplocampa testudinea, Monomorium pharaonis, Solenopsis geminata and Solenopsis invicta,
of the order Heteroptera, for example Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,
of the order Homoptera, for example Acyrthosiphon onobrychis, Adelges laricis, Aphidula nasturtii, Aphis craccivora, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolli, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefoli; Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii,
of the order Isoptera (termites), for example Calotermes flavicollis, Leucotermes flavipes, Reticulitermes lucifugus and Termes natalensis, and
of the order Orthoptera, for example Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus.

The compounds of the formula I and their salts are also suitable for controlling arachnids (Arachnoidea), such as Acaria (Acarina), for example of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni, Tarsonemidae spp. iwe Phytonemus pallidus and Polyphagotarsonemus latus, Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri and Oligonychus pratensis.

The compounds of the formula I and their salts are also suitable for controlling nematodes, for example root gall nematodes, for example Meloidogyne hapla, Meloidogyne incognita, Meloidogyne javanica, cyst-forming nematodes, for example Globodera rostochiensis, Heterodera avenae, Heterodera glycines, Heterodera schachtii, Heterodera trifolii, stem and leaf nematodes, for example Belonolaimus longicaudatus, Ditylenchus destructor, Ditylenchus dipsaci, Heliocotylenchus multicinctus, Longidorus elongatus, Radopholus similis, Rotylenchus robustus, Trichodorus primitivus, Tylenchorhynchus claytoni, Tylenchorhynchus dubius, Pratylenchus neglectus, Pratylenchus penetrans, Pratylenchus curvitatus and Pratylenchus goodeyi.

Accordingly, the invention also relates to a method for controlling the animal pests mentioned above wherein the animal plant pests or the plants, seeds, materials or the soil to be protected against attack by these harmful organisms are/is treated with an effective amount of the compounds of the formula I or salts thereof. Application can be both before and after attack of the materials, plants or seeds by the harmful organisms.

The pyrimidines of the general formula I, in particular the pyrimidines of the formula I according to the invention described in the above description as being preferred, and their pharmaceutically acceptable salts effectively inhibit the growth and/or the propagation of tumor cells, as can be demonstrated in standard tests with tumor cell lines, such as HeLa, MCF-7 and COLO 205. In particular, the pyrimidines of the formula I according to the invention generally have IC₅₀ values of <10⁻⁶ mol/l (i.e. <1 μM), preferably IC₅₀ values of <10⁻⁷ mol/l (i.e. <100 nM), for cell cycle inhibition in HeLa cells. Therefore, the pyrimidines of the formula I, in particular the pyrimidines of the formula I according to the invention described in the above description as being preferred, and their pharmaceutically acceptable salts are suitable for the treatment, inhibition or control of growth and/or propagation of tumor cells and the disorders associated therewith. Accordingly, they are suitable for cancer therapy in warm-blooded vertebrates, i.e. mammals and birds, in particular humans, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The pyrimidines of the formula I, in particular the pyrimidines of the formula I according to the invention described in the above description as being preferred, and their pharmaceutically acceptable salts are suitable for the therapy of cancer or cancerous disorders of the following organs: breast, lung, intestine, prostate, skin (melanoma), kidney, bladder, mouth, larynx, oesophagus, stomach, ovaries, pancreas, liver and brain.

Furthermore, the invention relates to the pharmaceutical use of the pyrimidine compound I and their pharmaceutically acceptable salts, in particular the pyrimidines of the formula I according to the invention described in the above description as being preferred, and their pharmaceutically acceptable salts, and especially their use in the manufacture of a medicament for the treatment of cancer.

Moreover, the invention relates to pharmaceutical compositions, comprising at least one pyrimidine compound of the formula I and/or one pharmaceutically acceptable salt thereof and optionally at least one suitable carrier. Among these, in particular pharmaceutical compositions comprising at least one (new) pyrimidine compound of the formula I according to the invention and/or one pharmaceutically acceptable salt thereof are preferred. Among these, pharmaceutical compositions comprising at least one pyrimidine compound of the formula I mentioned above as being preferred and/or a pharmaceutically acceptable salt thereof are also particularly preferred.

In addition to the pyrimidine compound I and/or its pharmaceutically acceptable salt, the pharmaceutical compositions according to the invention optionally comprise at least one suitable carrier. Suitable carriers are, for example, solvents, carriers, excipients, binders and the like customarily used for pharmaceutical formulations, which are described below in an exemplary manner for individual types of administration.

The compounds of the formula I according to the invention or the compounds of the formula I used according to the invention can be administered in a customary manner, for example orally, intravenously, intramuscularly or subcutaneously. For oral administration, the active compound can be mixed, for example, with an inert diluent or with an edible carrier; it can be embedded into a hard or soft gelatin capsule, it can be compressed to tablets or it can be mixed directly with the food/feed. The active compound can be mixed with excipients and administered in the form of indigestible tablets, buccal tablets, pastilles, pills, capsules, suspensions, potions, syrups and the like. Such preparations should contain at least 0.1% of active compound. The composition of the preparation may, of course, vary. It usually comprises from 2 to 60% by weight of active compound, based on the total weight of the preparation in question (dosage unit). Preferred preparations of the compound I according to the invention or of the compound of the formula I used according to the invention comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like. Capsules may furthermore comprise a liquid carrier. Other substances which modify the properties of the dosage unit may also be used. For example, tablets, pills and capsules may be coated with shellac, sugar or mixtures thereof. In addition to the active compound, syrups or potions may also comprise sugar (or other sweeteners), methyl- or propylparaben as preservative, a colorant and/or a flavour. The components of the active compound preparations must, of course, be pharmaceutically pure and nontoxic at the quantities employed. Furthermore, the active compounds can be formulated as preparations with a controlled release of active compound, for example as delayed-release preparations.

The active compounds can also be administered parenterally or intraperitoneally. Solutions or suspensions of the active compounds or their salts can be prepared with water using suitable wetting agents, such as hydroxypropylcellulose. Dispersions can also be prepared using glycerol, liquid polyethylene glycols and mixtures thereof in oils. Frequently, these preparations furthermore comprise a preservative to prevent the growth of microorganisms.

Preparations intended for injections comprise sterile aqueous solutions and dispersions and also sterile powders for preparing sterile solutions and dispersions. The preparation has to be sufficiently liquid for injection. It has to be stable under the preparation and storage conditions and it has to be protected against contamination by microorganisms. The carrier may be a solvent or a dispersion medium, for example, water, ethanol, a polyol (for example glycerol, propylene glycol or liquid polyethylene glycol), a mixture thereof and/or a vegetable oil.

### SYNTHESIS EXAMPLES

The procedure described in the following synthesis examples was used to prepare further compounds of the formula I by appropriate modification of the starting compounds.

### Example 1

### N-Methoxy-5-(2-fluoropyridin-3-yl)-4-methyl-6-(2,2,2-trifluoroethylamino)-2-pyrimidinecarboximidamide

### 1.1 4-Chloro-6-methyl-2-methylthiopyrimidine

At room temperature and with stirring, 0.1 g of the catalyst 1,1′-bis(diphenylphosphino)ferrocene]palladium(II) chloride/methylene chloride complex (the total amount was 1.05 g (1.28 mmol) of [1,1′-bis-(diphenylphosphino)ferrocene]palladium(II) chloride/methylene chloride complex) was added to 50.0 g (256 mmol) of 4,6-dichloro-2-methylthiopyrimidine in 350 ml of tetrahydrofuran. 86.0 ml (256 mmol) of a 3 molar methylmagnesium chloride solution in tetrahydrofuran were then added dropwise such that the reaction temperature remained at 25-30° C. At the same time, the remaining amount of catalyst was added a little at a time (per 10 ml of Grignard solution about 0.1 g). The mixture was then stirred at room temperature overnight, added at 15-20° C. to 500 ml of saturated ammonium chloride solution and stirred for another 15 minutes. The mixture was extracted three times with in each case 250 ml of methyl tert-butyl ether and the extract was dried over sodium sulfate and concentrated under reduced pressure. The yield was quantitative (45.7 g), and the crude product was directly processed further.

### 1.2 4-Methyl-2-methylthio-6-(2,2,2-trifluoroethylamino)pyrimidine

In an autoclave, 25.0 g (143 mmol) of 4-chloro-6-methyl-2-methylthiopyrimidine, suspended in 70.9 g (715 mmol) and 2,2,2-trifluoroethylamine were heated at 100° C. (external temperature) for 2 days. The reaction mixture was added to 400 ml of water and 500 ml of ethyl acetate, the pH was adjusted to 3 using hydrochloric acid and the organic phase was removed. Using aqueous sodium hydroxide solution, the aqueous phase was adjusted to a pH of 6 and the resulting precipitate was filtered off, washed with water and dried under reduced pressure, which gave 16.7 g of the title compound.

### 1.3 5-Iodo-4-methyl-2-methylthio-6-(2,2,2-trifluoroethylamino)pyrimidine

7.09 g (86.4 mmol) of sodium acetate were added to a solution of 16.7 g (17.4 mmol) of 4-methyl-2-methylthio-6-(2,2,2-trifluoroethylamino)pyrimidine in 66 ml of acetic acid, and the mixture was stirred at room temperature for 1 h. 12.95 g (79.8 mmol) of chloroiodide were then added dropwise, resulting in an increase of the reaction temperature to 35° C. With stirring, the mixture was allowed to cool to room temperature and poured into 400 ml of water, and 10% strength sodium thiosulfate solution was added until the mixture was colorless. The mixture was then extracted three times with in each case 120 ml of ethyl acetate and the combined extracts were dried over sodium sulfate and concentrated under reduced pressure. Purification by chromatography on silica gel 60 using cyclohexane/methyl tert-butyl ether gave 15.8 g of the title compound.

### 1.4 5-Iodo-4-methyl-2-methylsulfonyl-6-(2,2,2-trifluoroethylamino)pyrimidine

With stirring at 0-5° C., 21.45 g (87.0 mmol) of 70% strength 3-chloroperbenzoic acid were added a little at a time to 15.80 g (43.5 mmol) of 5-iodo-4-methyl-2-methylthio-6-(2,2,2-trifluoroethylamino)pyrimidine in 130 ml of methylene chloride, and the mixture was stirred 5° C. for 7 h and at room temperature for 16 h. The reaction mixture was concentrated under reduced pressure, suspended in 100 ml of ethyl acetate, washed three times with in each case 50 ml of saturated sodium bicarbonate solution, dried over sodium sulfate, concentrated under reduced pressure and purified by trituration with diisopropyl ether, which gave 13.4 g of the title compound.

### 1.5 2-Cyano-5-iodo-4-methyl-6-(2,2,2-trifluoroethylamino)pyrimidine

At room temperature and with stirring, 2.80 g (43.0 mmol) of potassium cyanide and 67 mg (0.25 mmol) of crown ether (18-crown-6) were added to 10.0 g (25.3 mmol) of 5-iodo-4-methyl-2-methylsulfonyl-6-(2,2,2-trifluoroethylamino)pyrimidine in 75 ml of acetonitrile, the mixture was stirred at room temperature for 1d, another 1.00 g of potassium cyanide and 67 mg of crown ether were added, the mixture was again stirred at room temperature for 1d and at 40° C. for 6 h, another 67 mg of crown ether were added and the mixture was stirred at 40° C. for 6 h bei and at room temperature for 3 d. The reaction mixture was concentrated under reduced pressure, taken up in ethyl acetate and water and extracted twice with ethyl acetate. The extracts were washed with water, dried over sodium sulfate and concentrated under reduced pressure, which gave 5.7 g of the title compound of melting point 103-105° C.

### 1.6 N-Methoxy-5-iodo-4-methyl-6-(2,2,2-trifluoroethylamino)-2-pyrimidinecarboximidamide

5.40 g (15.8 mmol) of 2-cyano-5-iodo-4-methyl-6-(2,2,2-trifluoroethylamino)pyrimidine were suspended in 50 ml of methanol, 38 mg (1.6 mmol) of lithium hydroxide were added at room temperature and with stirring under a nitrogen atmosphere and the mixture was stirred overnight. 1.58 g (18.9 mmol) of methoxyamine hydrochloride were added, and the mixture was stirred at room temperature overnight. The reaction mixture was concentrated under reduced pressure, added to methyl tert-butyl ether/water and acidified with hydrochloric acid. The product, which was obtained as the hydrochloride, was again transferred into methyl tert-butyl ether/water (1:1), and the pH was made weakly basic using sodium bicarbonate. The organic phase was separated off, the aqueous phase was extracted twice with methyl tert-butyl ether and the combined organic phases were washed twice with water, dried over sodium sulfate and concentrated under reduced pressure. For purification, the residue was triturated with diisopropyl ether, which gave 4.96 g of the title compound as light-yellow crystals of melting point 150-152° C.

### 1.7 N-Methoxy-5-(2-fluoropyridin-3-yl)-4-methyl-6-(2,2,2-trifluoroethylamino)-2-pyrimidinecarboximidamide

200 mg (0.51 mmol) of N-methoxy-5-iodo-4-methyl-6-(2,2,2-trifluoroethylamino)-2-pyrimidinecarboximidamide, 110 mg (0.77 mmol) of 3-fluoropyridin-3-yl-boronic acid, 18 mg (0.1 mmol) of palladium dichloride, 22 mg (0.08 mmol) of tri-tert-butylphosphine tetrafluoroborate and 16 mg (0.05 mmol) of tri-orthotolylphosphine were suspended in 2.5 ml of propionitrile, and at room temperature and with stirring 266 mg (2.06 mmol) of N-ethyl-N-diisopropylamine and 0.2 ml of water were added under a nitrogen atmosphere. The mixture was then heated under reflux (about 100° C.) for 6 h, taken up in methyl tert-butyl ether and washed with water. The aqueous phase was extracted twice with methyl tert-butyl ether and the combined organic phases were dried over sodium sulfate and concentrated under reduced pressure. The product was purified by MPLC on RP material using acetonitrile/water and then purified by chromatography on silica gel 60 using cyclohexane/ethyl acetate, which gave 30 mg of the title compound as an oil.

¹H-NMR (CDCl₃): δ=2.27 (s); 4.01 (m); 4.05 (s); 4.42 (m); 4.71 (br.); 5.45 (br.); 7.40 (m); 7.77 (m); 8.38 (d).

The compounds I listed in Table 1 can be prepared in an analogous manner.

TABLE 1
(I)

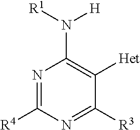

| | | | | | ¹H-NMR (CDCl₃) [ppm]/ |
|---|---|---|---|---|---|
| Ex | R¹ | R³ | R⁴ | Het | RT (HPLC/MS) |
| 1 | 2,2,2- | CH₃ | C(═NOCH₃)NH₂ | 2-fluoro- | δ = 2.27 (s); 4.01 (m); |
| | trifluoroethyl | | | pyridin-3-yl | 4.05 (s); 4.42 (m); 4.71 |
| | | | | | (br.); 5.45 (br.); 7.40 |
| | | | | | (m); 7.77 (m); 8.38 (d). |
| 2 | 2,2,2- | CH₃ | C(═NOCH₃)NH₂ | 2-fluoro-6- | δ = 2.25 (s); 2.60 (s); |
| | trifluoroethyl | | | methylpyridin- | 4.00 (m); 4.05 (s); 4.42 |
| | | | | 3-yl | (m); 4.72 (br.); 5.47 |
| | | | | | (br.); 7.23 (dd); 7.60 |
| | | | | | (dd). |

Ex: Example
RT: retention time. HPLC conditions: Merck ROD column, 50 × 4.6 mm. Gradient: acetonitrile with 0.1% trifluoroacetic acid/water with 0.1% trifluoroacetic acid; from 5% to 100% acetonitrile phase in 5 min.

### Use Examples

### Microtiter Test

The active compounds were formulated separately as a stock solution in dimethyl sulfoxide at a concentration of 10 000 ppm.

### Use Example 1

### Activity Against the Rice Blast Pathogen Pyricularia oryzae in the Microtiter Test

The stock solution is pipetted onto a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant and the fungus- and active compound-free blank value to determine the relative growth in % of the pathogens in the individual active compounds.

In this test, the sample which had been treated with 125 ppm of the compound from example 2 showed 10% relative growth of the pathogen.

1-31. (canceled)

32. A composition of the formula I and/or an agriculturally acceptable salt thereof 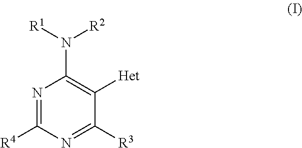
wherein
Het is a 5- or 6-membered aromatic heterocycle which has 1, 2, 3 or 4 heteroatoms selected from the group consisting of nitrogen, oxygen and sulfur as ring members, wherein the 5- or 6-membered heteroaromatic radical may have 1, 2, 3 or 4 identical or different substituents L, and wherein
L is selected from the group consisting of halogen; cyano; hydroxyl; cyanato (OCN); nitro; C₁-C₈-alkyl; C₂-C₁₀-alkenyl; C₂-C₁₀-alkynyl; C₁-C₆-haloalkyl; C₂-C₁₀-haloalkenyl; C₁-C₆-alkoxy; C₂-C₁₀-alkenyloxy; C₂-C₁₀-alkynyloxy; C₁-C₆-haloalkoxy; C₃-C₆-cycloalkyl; C₃-C₈-cycloalkenyl; C₃-C₆-cycloalkoxy; C₁-C₈-alkoximinoalkyl; C₂-C₁₀-alkenyloximinoalkyl; C₂-C₁₀-alkynyloximinoalkyl; C₂-C₁₀-alkynylcarbonyl; C₃-C₆-cycloalkylcarbonyl; NR⁵R⁶, NR⁵—C(═O)—R⁶; NR⁵—C(═S)—R⁶; S(═O)ₙA¹; C(═O)A²; C(═S)A²; a group —C(═N—OR⁷)A³; a group —C(═N—NR⁸R⁹)A⁴, phenyl and a five-, six-, seven-, eight-, nine- or ten-membered saturated, partially unsaturated or aromatic heterocycle which has one, two, three or four heteroatoms selected from the group consisting of O, N and S as ring members and in which phenyl and the heterocycle are unsubstituted or may have 1, 2, 3 or 4 substituents selected from the group consisting of halogen, nitro, cyano, OH, C₁-C₂-alkyl, C₁-C₂-haloalkyl, C₁-C₂-alkoxy, C₁-C₂-haloalkoxy, C₁-C₄-alkoxycarbonyl, C₁-C₄-alkylcarbonyl, amino, C₁-C₄-alkylamino and di-C₁-C₄-alkylamino; wherein
R⁵, R⁶, independently of one another, are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, wherein the 5 last-mentioned radicals may be partially or fully halogenated and/or may carry one, two, three or four radicals selected from the group consisting of cyano, C₁-C₄-alkoximino, C₂-C₄-alkenyloximino, C₂-C₄-alkynyloximino and C₁-C₄-alkoxy;
A¹ is hydrogen, hydroxyl, C₁-C₈-alkyl, amino, C₁-C₈-alkylamino or di-(C₁-C₈-alkyl)amino;
n is 0, 1 or 2;
A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A;
A³ and A⁴, independently of one another, are C₁-C₈-alkyl, C₂-C₈-alkenyl, C₁-C₈-haloalkyl, C₂-C₈-haloalkenyl, C₃-C₆-cycloalkyl, C₁-C₈-alkoxy, C₁-C₆-haloalkoxy, C₂-C₁₀-alkenyloxy, C₂-C₁₀-alkynyloxy or a group NR¹⁰R¹¹;
R⁷, R⁸, R⁹, R¹⁰ and R¹¹, independently of one another, are selected from the group consisting of hydrogen, C₁-C₆-alkyl, C₃-C₆-cycloalkyl, C₂-C₆-alkenyl and C₂-C₆-alkynyl, wherein the four last-mentioned radicals may have one, two, three, four, five or six radicals R^{a}; or
R⁸ and R⁹ and/or R¹⁰ and R¹¹, together with the nitrogen atom to which they are attached, form a four-, five- or six-membered saturated or partially unsaturated ring which may carry one, two, three or four substituents, independently of one another, selected from R^{a};
R^{a} is halogen, OH, C₁-C₈-alkyl or C₁-C₈-alkoxy;
R¹ is hydrogen, C₁-C₈-alkyl, C₃-C₈-cycloalkyl, C₅-C₁₀-bicycloalkyl, C₂-C₈-alkenyl, C₄-C₁₀-alkadienyl, C₃-C₆-cycloalkenyl, C₂-C₈-alkynyl, phenyl, naphthyl or a five- or six-membered saturated, partially unsaturated or aromatic heterocycle which is attached via carbon and which has one, two, three or four heteroatoms selected from the group consisting of O, N and S as ring members;
R² is R¹ or one of the following radicals: NH₂, C₁-C₈-alkoxy, C₃-C₈-cycloalkoxy, C₂-C₈-alkenyloxy, C₂-C₈-alkynyloxy, C₁-C₈-alkylamino or di-C₁-C₈-alkylamino;
wherein the radicals R¹ and R² that are different from hydrogen may also be partially or fully halogenated and/or may carry one, two, three or four identical or different groups R²¹:
R²¹ is cyano, nitro, hydroxyl, carboxyl, C₁-C₆-alkylcarbonyl, C₃-C₆-cycloalkyl, C₁-C₆-alkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₃-C₈-cycloalkenyl, C₂-C₆-alkenyloxy, C₂-C₆-alkynyl, C₃-C₆-alkynyloxy, C₃-C₆-cycloalkoxy, C₃-C₆-cycloalkenyloxy, oxy-C₁-C₃-alkyleneoxy, phenyl, naphthyl, or a five-, six-, seven-, eight-, nine- or ten-membered saturated, partially unsaturated or aromatic heterocycle which has one, two, three or four heteroatoms from the group consisting of O, N and S as ring members,
wherein the aliphatic, alicyclic, heterocyclic and aromatic groups in R²¹ may be partially or fully halogenated or may carry one, two or three groups R²²:
R²² is cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, or dialkylaminothiocarbonyl, wherein the alkyl groups in these radicals contain 1 to 6 carbon atoms and the alkenyl, alkadienyl or alkynyl groups in these radicals contain 2 to 8 carbon atoms;
cycloalkyl, bicycloalkyl, cycloalkoxy, heterocyclyl, or heterocyclyloxy, wherein the cyclic systems contain 3 to 10 ring members;
aryl, aryloxy, arylthio, aryl-C₁-C₆-alkoxy, aryl-C₁-C₆-alkyl, hetaryl, hetaryloxy, or hetarylthio, wherein the aryl radicals preferably contain 6, 7, 8, 9 or 10 ring members and the hetaryl radicals contain 5 or 6 ring members and wherein the cyclic systems may be partially or fully halogenated or substituted by alkyl or haloalkyl groups;
R¹ and R², together with the nitrogen atom to which they are attached, may also form a five- or six-membered saturated, partially unsaturated or aromatic heterocycle which is attached via N and which may have one, two or three further heteroatoms selected from the group consisting of O, N and S as ring members and/or may carry one or more substituents selected from the group consisting of halogen, oxo, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, and C₃-C₆-haloalkenyloxy and/or wherein two substituents attached to adjacent ring atoms may be C₁-C₆-alkylene, oxy-C₂-C₄-alkylene or oxy-C₁-C₃-alkyleneoxy;
R³ is hydrogen, OH, halogen, cyano, NR³¹R³², C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, where the 7 last-mentioned radicals may be partially or fully halogenated and/or may carry one, two or three substituents selected from the group consisting of nitro, cyano, OH, C₁-C₂-alkoxy, C₁-C₄-alkoxycarbonyl, amino, C₁-C₄-alkylamino and di-C₁-C₄-alkylamino, wherein R³¹ has one of the meanings given for R⁵ and R³² has one of the meanings given for R⁶;
R⁴ is halogen, cyano, hydroxyl, mercapto, N₃, C₁-C₈-alkyl, C₂-C₈-alkenyl, C₂-C₈-alkynyl, C₁-C₆-haloalkyl, C₁-C₆-alkoxy, C₃-C₈-alkenyloxy, C₃-C₈-alkynyloxy, C₁-C₆-haloalkoxy, C₁-C₆-alkylthio, C₃-C₈-alkenylthio, C₃-C₈-alkynylthio, C₁-C₆-haloalkylthio, or is a radical of the formula C(═Z)OR⁴¹, C(═Z)NR⁴²R⁴³, C(═Z)NR⁴⁴—NR⁴²R⁴³, C(═Z)R⁴⁵, CR⁴⁶R⁴⁷—OR⁴⁸, CR⁴⁶R⁴⁷—NR⁴²R⁴³, ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR^{52a}(N═CR⁴⁹R⁵⁰), NR⁵²NR⁴²R⁴³NR⁵²OR⁴¹ or C(═N—X—R⁴⁵)SR⁴¹, wherein
Z is O, S, NR⁵³, NOR⁵⁴ or N—NR⁵⁵R⁵⁶;
X is a chemical bond, oxygen, a carbonyl group, a group NR⁵² or one of the following groups: —(C═O)—NH— or —(C═O)—O—, where the carbonyl group is attached to the nitrogen atom;
R⁴¹, R⁴², R⁴³, R⁴⁴, R⁴⁵, R⁴⁶, R⁴⁷, R⁴⁸, R⁴⁹, R⁵⁰, R⁵¹, R⁵², R^{52a}, R⁵³, R⁵⁴, R⁵⁵ and R⁵⁶, independently of one another, comprise hydrogen, C₁-C₆-alkyl, C₂-C₆-alkenyl, C₂-C₆-alkynyl, C₃-C₈-cycloalkyl or C₄-C₈-cycloalkenyl;
R^{43a} has one of the meanings given for R⁴¹ except for hydrogen;
R⁴², R⁴⁸ and R⁵², independently, further comprise —CO—R⁴⁵;
R⁴² further comprises —CO—OR⁴¹ or —CO—NR⁴³R^{43b}, where R^{43b} has one of the meanings given for R⁴¹;
optionally, R⁴² and R⁴³, together, comprise a C₃-C₆-alkylene group, wherein the C₃-C₆-alkylene group optionally is interrupted by an oxygen atom or has a double bond;
optionally, R⁴⁹ and R⁵⁰, together, comprise a C₃-C₆-alkylene group, wherein the C₃-C₆-alkylene group optionally is interrupted by an oxygen atom or has a double bond;
R⁵⁰ further comprises a radical of the formula A-CO—OR⁴¹ or —CO—NR⁴³R^{43b} wherein A is C₁-C₄-alkylene; and
R⁵¹ further comprises a group of the formula NR⁴²R⁴³, N═CR⁴⁹R⁵⁰ or N═C(R⁴⁵)NR⁴²R⁴³;
wherein the aliphatic or alicyclic groups of the radical definitions of R⁴¹-R⁵⁶ may be partially or fully halogenated and/or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, or C₃-C₆-cycloalkenyloxy.

33. The composition of claim 32, wherein at least one of the radicals R¹ and R² is different from hydrogen and wherein R³ is not hydrogen or C₁-C₈-alkyl if R⁴ is chlorine, NH₂, methyl, or their salts.

34. A compound for controlling phytopathogenic fungi, comprising at least one composition of formula I as claimed in claim 32 and/or an agriculturally acceptable salt thereof and at least one solid or liquid carrier.

35. A method of controlling plant-damaging fungi, comprising treating the fungi or a material to be protected against the fungi with an effective amount of a compound comprising the composition of formula I as claimed in claim 32 and/or an agriculturally useful salt thereof.

36. The method of claim 35, wherein R⁴ is selected from the group consisting of halogen, N₃, CN, C(═Z)OR⁴¹, C(═Z)NR⁴²R⁴³, C(═Z)NR⁴⁴—NR⁴²R⁴³, C(═Z)R⁴⁵, ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR⁵²(N═CR⁴⁹R⁵⁰), NR⁵²NR⁴²R⁴³, NR⁵²OR⁴¹ and C(═N—X—R⁴⁵)SR⁴¹.

37. The method of claim 36, wherein R⁴ is selected from the group consisting of CN, C(═Z)OR⁴¹, C(═Z)NR⁴²R⁴³, C(═Z)NR⁴⁴—NR⁴²R⁴³, C(═Z)R⁴⁵ and C(═N—X—R⁴⁵)SR⁴¹.

38. The method of claim 35, wherein R⁴ is selected from the group consisting of CN, C(═O)OR⁴¹, C(═O)NR⁴²R⁴³, C(═NOR⁵⁴)NR⁴²R⁴³, C(═O)NR⁴⁴—NR⁴²R⁴³, C(═N—OR⁴⁵)SR⁴¹ and C(═N—R⁴⁵)SR⁴¹.

39. The method of claim 35, wherein R⁴ is selected from the group consisting of ON(═CR⁴⁹R⁵⁰), O—C(═Z)R⁴⁵, NR⁴²R^{43a}, NR⁵¹(C(═Z)R⁴⁵), NR⁵¹(C(═Z)OR⁴¹), NR⁵¹(C(═Z)—NR⁴²R⁴³), NR⁵²(N═CR⁴⁹R⁵⁰), NR⁵²NR⁴²R⁴³ and NR⁵²OR⁴¹.

40. The method of claim 39, wherein R⁴ is selected from the group consisting of ON(═CR⁴⁹R⁵⁰), NR⁵¹(C(═O)R⁴⁵), NR⁵¹(C(═O)OR⁴¹), NR⁵¹(C(═O)—NR⁴²R⁴³), NR⁵²(N═CR⁴⁹R⁵⁰) and NR⁵²OR⁴¹.

41. The method of claim 35, wherein R¹ and R² are as defined below:
R¹ is C₁-C₆-alkyl, C₂-C₆-alkenyl, C₂-C₆-alkynyl, C₃-C₆-cycloalkyl, which may be mono-, di-, tri- or tetrasubstituted by halogen and/or C₁-C₄-alkyl, or C₁-C₈-haloalkyl, and
R² is hydrogen or C₁-C₄-alkyl; or
R¹ and R², together with the nitrogen atom to which they are attached, may also form a five- or six-membered saturated, monounsaturated or aromatic heterocycle which may carry one or two substituents selected from the group consisting of halogen, C₁-C₆-alkyl and C₁-C₆-haloalkyl.

42. The method of claim 35, wherein R³ is halogen, cyano, C₁-C₄-alkyl, C₁-C₄-haloalkyl, C₁-C₄-alkoxy or C₁-C₂-haloalkoxy.

43. The method of claim 35, wherein Het is attached in the ortho-position to at least one of the ring heteroatoms.

44. The method of claim 35, wherein Het has at least one radical L which is attached in the ortho-position to the atom of Het which is attached to the pyrimidine ring.

45. The method of claim 35, wherein Het is a 5-membered heteroaromatic radical which has at least one nitrogen atom or one nitrogen atom and 1 or 2 further heteroatoms selected from the group consisting of O, S and N as ring members and which is unsubstituted or carries 1, 2 or 3 substituents L.

46. The method of claim 45, wherein Het is selected from the group consisting of pyrrolyl, pyrazolyl, imidazolyl, 1,2,3-triazolyl, 1,2,4-triazolyl, oxazolyl, thiazolyl, isoxazolyl and isothiazolyl, wherein Het is unsubstituted or carries 1, 2 or 3 substituents L.

47. The method of claim 46, wherein Het is pyrazol-1-yl which is unsubstituted or has 1, 2 or 3 substituents L.

48. The method of claim 46, wherein Het is thiazolyl-2-yl which is unsubstituted or has 1, 2 or 3 substituents L.

49. The method of claim 35, wherein Het is a 6-membered heteroaromatic radical which has 1, 2 or 3 nitrogen atoms as ring members and which is unsubstituted or carries 1, 2, 3 or 4 substituents L.

50. The method of claim 49, wherein Het is selected from the group consisting of pyridinyl and pyrimidinyl, wherein Het is unsubstituted or carries 1, 2 or 3 substituents L.

51. The method of claim 50, wherein Het is 2-pyridinyl which is unsubstituted or carries 1, 2 or 3 substituents L.

52. The method of claim 51, wherein one of the substituents L is located in the 5-position of the pyridinyl ring.

53. The method of claim 51, wherein one of the substituents L is located in the 3-position of the pyridinyl ring.

54. The method of claim 50, wherein Het is 3-pyridinyl which carries 1, 2 or 3 substituents L, wherein one of the substituents L is located in the 2-position or the 4-position of the pyridinyl ring.

55. The method of claim 35, wherein Het has 1, 2 or 3 substituents L, independently of one another, selected from the group consisting of halogen, cyano, nitro, NH₂, C₁-C₆-alkylamino, di-C₁-C₆-alkylamino, C₁-C₆-alkyl, C₁-C₆-haloalkyl, C₁-C₆-alkoxy, C₁-C₆-alkylamino, di-C₁-C₆-alkylamino, NH—C(O)—C₁-C₆-alkyl, a group C(S)A² and a group C(O)A².

56. The method of claim 35, comprising treating the fungi or the materials to be protected against fungal attack with an effective amount of more than one compound having the composition of formula I and/or an agriculturally acceptable salt thereof.

57. A seed, comprising at least one 5-hetaryl-4-aminopyrimidine of the formula I as claimed in claim 32 or an agriculturally acceptable salt thereof.

58. A pharmaceutical composition, comprising at least one 5-hetaryl-4-aminopyrimidine of the formula I and/or a pharmaceutically acceptable salt thereof and a pharmaceutically acceptable carrier 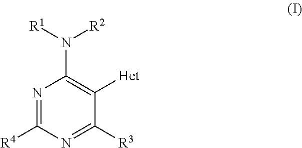
wherein
Het is a 5- or 6-membered aromatic heterocycle which has 1, 2, 3 or 4 heteroatoms selected from the group consisting of nitrogen, oxygen and sulfur as ring members, wherein the 5- or 6-membered heteroaromatic radical may have 1, 2, 3 or 4 identical or different substituents L, and wherein
L is selected from the group consisting of halogen; cyano; hydroxyl; cyanato (OCN); nitro; C₁-C₈-alkyl; C₂-C₁₀-alkenyl; C₂-C₁₀-alkynyl; C₁-C₆-haloalkyl; C₂-C₁₀-haloalkenyl; C₁-C₆-alkoxy; C₂-C₁₀-alkenyloxy; C₂-C₁₀-alkynyloxy; C₁-C₆-haloalkoxy; C₃-C₆-cycloalkyl; C₃-C₈-cycloalkenyl; C₃-C₆-cycloalkoxy; C₁-C₈-alkoximinoalkyl; C₂-C₁₀-alkenyloximinoalkyl; C₂-C₁₀-alkynyloximinoalkyl; C₂-C₁₀-alkynylcarbonyl; C₃-C₆-cycloalkylcarbonyl; NR⁵R⁶, NR⁵—C(═O)—R⁶; NR⁵—C(═S)—R⁶; S(═O)ₙA¹; C(═O)A²; C(═S)A²; a group —C(═N—OR⁷)A³; a group —C(═N—NR⁸R⁹)A⁴, phenyl and a five-, six-, seven-, eight-, nine- or ten-membered saturated, partially unsaturated or aromatic heterocycle which has one, two, three or four heteroatoms selected from the group consisting of O, N and S as ring members and in which phenyl and the heterocycle are unsubstituted or may have 1, 2, 3 or 4 substituents selected from the group consisting of halogen, nitro, cyano, OH, C₁-C₂-alkyl, C₁-C₂-haloalkyl, C₁-C₂-alkoxy, C₁-C₂-haloalkoxy, C₁-C₄-alkoxycarbonyl, C₁-C₄-alkylcarbonyl, amino, C₁-C₄-alkylamino and di-C₁-C₄-alkylamino; wherein
R⁵, R⁶, independently of one another, are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, wherein the 5 last-mentioned radicals may be partially or fully halogenated and/or may carry one, two, three or four radicals selected from the group consisting of cyano, C₁-C₄-alkoximino, C₂-C₄-alkenyloximino, C₂-C₄-alkynyloximino and C₁-C₄-alkoxy;
A¹ is hydrogen, hydroxyl, C₁-C₈-alkyl, amino, C₁-C₈-alkylamino or di-(C₁-C₈-alkyl)amino;
n is 0, 1 or 2;
A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹;
A³ and A⁴, independently of one another, are C₁-C₈-alkyl, C₂-C₈-alkenyl, C₁-C₈-haloalkyl, C₂-C₈-haloalkenyl, C₃-C₆-cycloalkyl, C₁-C₈-alkoxy, C₁-C₆-haloalkoxy, C₂-C₁₀-alkenyloxy, C₂-C₁₀-alkynyloxy or a group NR¹⁰R¹¹;
R⁷, R⁸, R⁹, R¹⁰ and R¹¹, independently of one another, are selected from the group consisting of hydrogen, C₁-C₆-alkyl, C₃-C₆-cycloalkyl, C₂-C₆-alkenyl and C₂-C₆-alkynyl, wherein the four last-mentioned radicals may have one, two, three, four, five or six radicals R^{a}; or
R⁸ and R⁹ and/or R¹⁰ and R¹¹, together with the nitrogen atom to which they are attached, form a four-, five- or six-membered saturated or partially unsaturated ring which may carry one, two, three or four substituents, independently of one another, selected from R^{a};
R^{a} is halogen, OH, C₁-C₈-alkyl or C₁-C₈-alkoxy;
R¹ is hydrogen, C₁-C₈-alkyl, C₃-C₈-cycloalkyl, C₅-C₁₀-bicycloalkyl, C₂-C₈-alkenyl, C₄-C₁₀-alkadienyl, C₃-C₆-cycloalkenyl, C₂-C₈-alkynyl, phenyl, naphthyl or a five- or six-membered saturated, partially unsaturated or aromatic heterocycle which is attached via carbon and which has one, two, three or four heteroatoms selected from the group consisting of O, N and S as ring members;
R² is R¹ or one of the following radicals: NH₂, C₁-C₈-alkoxy, C₃-C₈-cycloalkoxy, C₂-C₈-alkenyloxy, C₂-C₈-alkynyloxy, C₁-C₈-alkylamino or di-C₁-C₈-alkylamino;
wherein the radicals R¹ and R² that are different from hydrogen may also be partially or fully halogenated and/or may carry one, two, three or four identical or different groups R²¹:
R²¹ is cyano, nitro, hydroxyl, carboxyl, C₁-C₆-alkylcarbonyl, C₃-C₆-cycloalkyl, C₁-C₆-alkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₃-C₈-cycloalkenyl, C₂-C₆-alkenyloxy, C₂-C₆-alkynyl, C₃-C₆-alkynyloxy, C₃-C₆-cycloalkoxy, C₃-C₆-cycloalkenyloxy, oxy-C₁-C₃-alkyleneoxy, phenyl, naphthyl, or a five-, six-, seven-, eight-, nine- or ten-membered saturated, partially unsaturated or aromatic heterocycle which has one, two, three or four heteroatoms from the group consisting of O, N and S as ring members,
wherein the aliphatic, alicyclic, heterocyclic and aromatic groups in R²¹ may be partially or fully halogenated or may carry one, two or three groups R²²:
R²² is cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, or dialkylaminothiocarbonyl, wherein the alkyl groups in these radicals contain 1 to 6 carbon atoms and the alkenyl, alkadienyl or alkynyl groups in these radicals contain 2 to 8 carbon atoms;
cycloalkyl, bicycloalkyl, cycloalkoxy, heterocyclyl, or heterocyclyloxy, wherein the cyclic systems contain 3 to 10 ring members;
aryl, aryloxy, arylthio, aryl-C₁-C₆-alkoxy, aryl-C₁-C₆-alkyl, hetaryl, hetaryloxy, or hetarylthio, wherein the aryl radicals preferably contain 6, 7, 8, 9 or 10 ring members and the hetaryl radicals contain 5 or 6 ring members and wherein the cyclic systems may be partially or fully halogenated or substituted by alkyl or haloalkyl groups;
R¹ and R², together with the nitrogen atom to which they are attached, may also form a five- or six-membered saturated, partially unsaturated or aromatic heterocycle which is attached via N and which may have one, two or three further heteroatoms selected from the group consisting of O, N and S as ring members and/or may carry one or more substituents selected from the group consisting of halogen, oxo, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, and C₃-C₆-haloalkenyloxy and/or wherein two substituents attached to adjacent ring atoms may be C₁-C₆-alkylene, oxy-C₂-C₄-alkylene or oxy-C₁-C₃-alkyleneoxy;
R³ is hydrogen, OH, halogen, cyano, NR³¹R³², C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, where the 7 last-mentioned radicals may be partially or fully halogenated and/or may carry one, two or three substituents selected from the group consisting of nitro, cyano, OH, C₁-C₂-alkoxy, C₁-C₄-alkoxycarbonyl, amino, C₁-C₄-alkylamino and di-C₁-C₄-alkylamino, wherein R³¹ has one of the meanings given for R⁵ and R³² has one of the meanings given for R⁶;
R⁴ is halogen, cyano, hydroxyl, mercapto, N₃, C₁-C₈-alkyl, C₂-C₈-alkenyl, C₂-C₈-alkynyl, C₁-C₆-haloalkyl, C₁-C₆-alkoxy, C₃-C₈-alkenyloxy, C₃-C₈-alkynyloxy, C₁-C₆-haloalkoxy, C₁-C₆-alkylthio, C₃-C₈-alkenylthio, C₃-C₈-alkynylthio, C₁-C₆-haloalkylthio, or is a radical of the formula C(═Z)OR⁴¹, C(═Z)NR⁴²R⁴³, C(═Z)NR⁴⁴—NR⁴²R⁴³, C(═Z)R⁴⁵, CR⁴⁶R⁴⁷—OR⁴⁸, CR⁴⁶R⁴⁷—NR⁴²R⁴³, ON(═CR⁴⁹R⁵⁰), O—C(═Z)R⁴⁵, NR⁴²R^{43a}, NR⁵¹(C(═Z)R⁴⁵), NR⁵(C(═Z)OR⁴¹), NR⁵¹(C(═Z)—NR⁴²R⁴³), NR^{52a}(N═CR⁴⁹R⁵⁰), NR⁵²NR⁴²R⁴³NR⁵²OR⁴¹ or C(═N—X—R⁴⁵)SR⁴¹, wherein
Z is O, S, NR⁵³, NOR⁵⁴ or N—NR⁵⁵R⁵⁶;
X is a chemical bond, oxygen, a carbonyl group, a group NR⁵² or one of the following groups: —(C═O)—NH— or —(C═O)—O—, where the carbonyl group is attached to the nitrogen atom;
R⁴¹, R⁴², R⁴³, R⁴⁴, R⁴⁵, R⁴⁶, R⁴⁷, R⁴⁸, R⁴⁹, R⁵⁰, R⁵¹, R⁵², R^{52a}, R⁵³, R⁵⁴, R⁵⁵ and R⁵⁶, independently of one another, comprise hydrogen, C₁-C₆-alkyl, C₂-C₆-alkenyl, C₂-C₆-alkynyl, C₃-C₈-cycloalkyl or C₄-C₈-cycloalkenyl;
R^{43a} has one of the meanings given for R⁴¹ except for hydrogen;
R⁴², R⁴⁸ and R⁵², independently, further comprise —CO—R⁴⁵;
R⁴² further comprises —CO—OR⁴¹ or —CO—NR⁴³R^{43b}, where R^{43b} has one of the meanings given for R⁴¹;
optionally, R⁴² and R⁴³, together, comprise a C₃-C₆-alkylene group, wherein the C₃-C₆-alkylene group optionally is interrupted by an oxygen atom or has a double bond;
optionally, R⁴⁹ and R⁵⁰, together, comprise a C₃-C₆-alkylene group, wherein the C₃-C₆-alkylene group optionally is interrupted by an oxygen atom or has a double bond;
R⁵⁰ further comprises a radical of the formula A-CO—OR⁴¹ or —CO—NR⁴³R^{43b} wherein A is C₁-C₄-alkylene; and
R⁵¹ further comprises a group of the formula NR⁴²R⁴³, N═CR⁴⁹R⁵⁰ or N═C(R⁴⁵)NR⁴²R⁴³;
wherein the aliphatic or alicyclic groups of the radical definitions of R⁴¹-R⁵⁶ may be partially or fully halogenated and/or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, or C₃-C₆-cycloalkenyloxy.

59. A method for cancer treatment in mammals, comprising administering to a mammal in need thereof an effective amount of a 5-hetaryl-4-aminopyrimidine of the formula I as defined in claim 58 and/or a pharmaceutically acceptable salt thereof.
